(19) United States
(12) Patent Application Publication
(10) Pub. No.: US 2019/0054468 A1
(43) Pub. Date: Feb. 21, 2019

(54) DEVICES, SYSTEMS AND METHODS FOR SAMPLE DETECTION

(71) Applicant: University of Virginia Patent Foundation, Charlottesville, VA (US)

(72) Inventors: James P. LANDERS, Charlottesville, VA (US); Kimberly Renee JACKSON, Atlanta, GA (US); Daniel MILLS, Charlottesville, VA (US); Gavin T. GARNER, Charlottesville, VA (US); Jacquelyn A. DuVall, Raleigh, NC (US); Jingyi LI, Charlottesville, VA (US)

(21) Appl. No.: 15/770,413

(22) PCT Filed: Oct. 21, 2016
(86) PCT No.: PCT/US2016/058263

§ 371 Date: Apr. 23, 2018

Related U.S. Application Data
(60) Provisional application No. 62/245,592, filed on Oct. 23, 2015.
(60) Provisional application No. 62/246,789, filed on Oct. 27, 2015.
(60) Provisional application No. 62/352,264, filed on Jun. 20, 2016.

(51) Int. Cl. B01L 3/00; C12Q 1/6809; C12Q 1/6816; C12Q 1/6827; G01N 33/52; G01N 33/543; G01N 33/68; G01N 35/00

(52) U.S. Cl. CPC B01L 3/502738; C12Q 2563/149; C12Q 1/6816; C12Q 1/6827; G01N 33/52; G01N 33/54326; G01N 33/68; G01N 35/00069; B01L 2200/0647; B01L 2300/0803; B01L 2300/0867; B01L 2400/0409; B01L 2400/043; B01L 2400/0633; C12Q 2563/143; C12Q 1/6809

(57) ABSTRACT

This disclosure provides for apparatuses, systems and methods for in vitro sample detection. For example in one embodiment, this disclosure provides an automated Pe-toner microfluidic device (and related method) on a centrifugal platform for DNA sample lysis and DNA extraction. A second embodiment provides a system and method for qualitative detection, quantification, and real-time monitoring of nucleic acid amplification products using magnetic bead aggregation inhibition. A third embodiment provides a platform for simultaneous detection of mRNA markers from blood, cell-free semen, sperm, saliva, and vaginal fluid. The third embodiment comprises a system and method that provide for simple, rapid, and fluorescence-free detection of body fluids using mRNA marker amplification and optical detection for mRNA marker analysis with a smart phone with image analysis.

### CROSS-REFERENCE TO RELATED APPLICATIONS

This application claims the benefit of the filing date of U.S. application Ser. No. 62/245,592, filed on Oct. 23, 2015, U.S. application Ser. No. 62/246,789, filed on Oct. 27, 2015, and U.S. application Ser. No. 62/352,264, filed Jun. 20, 2016, the disclosures of which are incorporated by reference herein.

### BACKGROUND

Forensic laboratories heavily rely on successful liberation of the DNA from cells followed by DNA purification, as these are precursors to obtaining the primary form of DNA evidence used in criminal cases, or short tandem repeat (STR) profiles. Most commonly, buccal swabs are substrates used for DNA processing as analysts search for traces of DNA on submitted pieces of evidence (Butler et al., 2011). At the present time, small cuttings are taken from each sample and chemically lysed in a chaotropic solution at an elevated temperature (56° C.) for a minimum of 30 minutes. During this step, the cells are desorbed from the swab and cells disrupted, causing the release of DNA into the solution. Once finished, these samples are transferred to a robot instrument, such as a Qiagen EZ1, which performs a solid-phase extraction in approximately 20 minutes. A conventional solid phase extraction requires 3 basic steps: initial binding of the DNA to silica-coated magnetic particles, washing of the beads in an alcohol solution to precipitate the DNA and solubilize contaminants to eliminate any non-specific binding, and elution of the DNA from the silica particles to yield PCR-ready DNA that can be used directly for post processing. External magnets, built into the instrument, move magnetic silica-coated particles through the solution for the initial binding and final elution of the DNA.

Although the lysis method and extraction instruments are fully validated, providing reproducible results for casework, both lend themselves to several limitations such as inefficiency of sample use and non-portability. The maximum volume usable in post processing (i.e., DNA quantitation and amplification) is 12 μL, which accounts for only 6% of the total volume provided from an extraction instrument. Therefore, 90% of the extracted sample volume is more than likely never used, as the remaining 4% may be used for retesting of the sample.

A stand-alone microfluidic device for sample lysis DNA extraction would allow for decreased sample and reagent volumes, reducing the cost per sample and minimizing wasted reagents. Current microfluidic devices, however, require high sample volumes that are not forensically relevant (≥100 μL), require high speeds and complex valving that limits portability, and has not demonstrated multiplexed amplification required for forensic lab DNA testing (Cho et al., 2007; Gan et al., 2014; Karle et al., 2010; Chung et al., 2004).

### SUMMARY

In one embodiment, this disclosure provides a polyethylene terephthalate (Pe) device that performs DNA lysis and extractions on samples in vitro. The samples can be whole blood and cuttings from buccal swabs, for example. In one embodiment, an automated Pe-toner microfluidic device (and related method) on a centrifugal platform for DNA sample lysis and DNA extraction is provided. In one embodiment, DNA lysis and extraction on a polyethylene terephthalate (Pe) rotationally-driven microdevice and related method thereof are provided.

In one embodiment, a system and method for qualitative detection, quantification, and real-time monitoring of nucleic acid amplification products using magnetic bead aggregation inhibition is provided. The presence of amplification products may be able to inhibit the aggregation of silica-coated magnetic beads in the presence of long, generic DNA strands e.g., the cutoff for aggregation is around 10 Kb, rendering the assay in the absence of amplification products incapable of detecting the presence of shorter fragments (e.g., PCR or LAMP amplification products). As a result of the presence of amplification products, referred to as Product-Inhibited Aggregation (PIA), the extent of aggregation inhibition (AI) can be used for qualitative detection, quantification of an amplicon present in an in vitro sample, and/or monitoring of an amplification reaction in a real-time manner.

In one embodiment, an inexpensive, fluorescence-free detection method (and related system) is provided that incorporates a simple operating procedure with a high-throughout (e.g., using a 96-well plate) platform for simultaneous detection of mRNA markers from in vitro samples of blood, cell-free semen, sperm, saliva, and vaginal fluid. The method (and related system) does not involve thermocycling, uses a simple dye for colorimetric read-out and a smart phone as a detector, which provides an accelerated sample-to-answer method for mRNA with high specificity and sensitivity (single copies of RNA) and unparalleled bandwidth (5 fluids) for body fluid ID. A single temperature isothermal amplification targets mRNA sequences unique to each body fluid and, when present, activates an embedded dye for visible colorimetric change. Full BFI analysis of 23 samples was conducted in under 3 hours using smart phone optical detection and analysis. The efficacy of the method is directly applicable to sexual assault analysis challenges in the United States. Thus, a system and method provide for simple, rapid, and fluorescence-free detection of body fluids using mRNA marker amplification and optical detection for mRNA marker analysis with a smart phone with image analysis.

### BRIEF DESCRIPTION OF THE FIGURES

FIG. 1. On-chip lysis design with labeled sections.

FIG. 2. Illustration of an exemplary device with labeled domains for dynamic solid-phase extraction (dSPE).

FIG. 3. Illustrated overlay of the different layers of polyethylene (Pe) material to form chambers and connecting channels for dSPE.

FIG. 4. Setup for dSPE rotationally-driven platform.

FIG. 4A. Is a perspective view of various components of the dSPE rotationally-driven platform of FIG. 4.

FIGS. 5A and 5B. Physical stop valves on the dSPE microfluidic device including a top and side-view 3D illustration of the stop valve used for fluidic control.

FIG. 6. Illustrations of the stop valve in an open (top) and closed (bottom) state.

FIG. 7. Comparison of the strength of different adhesives for the stop valve.

FIG. 8. Illustration of loaded dSPE device, ready for extraction, with accompanying description of magnetic mixing for initial binding and final elution of DNA (43.2° C. magnetic sweeping, 9 mm height differential, 500 seconds).

FIG. 9. Representation of the steps for dSPE on chip. Step 1: IPA wash (300 RPM, 120 seconds) following initial mixing to bind DNA. Step 2: TE buffer wash (400 RPM, 10\5 seconds). Step 3: Load elution buffer (700 RPM, 5 seconds) and mix to elute DNA. Step 4: Transfer elution buffer to separate chamber for post processing (800 RPM, 5 seconds). Alternatively, the following spin protocol may be used” 310 RPM for 110 seconds, 450 RPM for 10 seconds, 500 RPM for 1 second and 1000 RPM for 2 seconds.

FIG. 10. Comparison of on-chip dSPE chip extractions to other extraction methods: dSPE via manual mixing, and a conventional Qiagen extraction. Note: * Indicates multiple elutions from the extraction.

FIG. 11. PCR amplification of β-globin from an on-chip extraction.

FIG. 12. Full STR profile obtained from an on-chip extraction.

FIG. 13. Comparison of an on-chip extraction to a conventional column extraction.

FIG. 14. Determination of chamber volumes and deviations between volumes of sample extraction chambers.

FIG. 15. Front view illustration of the DNA extraction design on a PeT device with highlighted passive valve features for improved automation.

FIG. 16. On-chip extraction reproducibility study compared to Qiagen EZ1 instrument. Replicate on-chip extraction efficiency and STR profile comparisons to a conventionally used Qiagen EZ1 instrumental method.

FIG. 17. STR profile comparison of on-chip extractions and a conventional Qiagen EZ1 extraction method for multiple donors at various concentrations to realize the full applicable DNA bandwidth.

FIG. 18. Electropherogram comparison of extracted DNA samples. Comparison of DNA samples (about 2 ng/μL) from Qiagen EZ1 extraction and an on-chip extraction with a reagent blank control.

FIG. 19. Quantitative measurements of non-bonded DNA after an initial DNA-bead binding step with corresponding measurements of extracted DNA after full extractions at various GuHCl concentrations.

FIG. 20. Validation of lysis method for forensic samples.

FIG. 21. Validation of lysis method for forensic samples.

FIG. 22. Electropherogram showing successful DNA extraction of β-globin from DNA collected from a whole-blood DNA extraction, as confirmed by a positive tube amplification control.

FIG. 23. STR profiles from an on-chip extraction compared to a conventional extraction method.

FIG. 24. Left: On-Chip extraction from whole blood compared to a conventional Qiagen extraction. Right: On-chip extraction from buccal swab cutting compared to a conventional Qiagen EZ1 instrument.

FIG. 25. Qualitative PIA detection for LAMP and PCR amplification products. PIA response shown in presence and absence of RT-LAMP product (left graph), and PCR product of various lengths (right graph).

FIG. 26. Real-time monitoring of RT-LAMP (left) and PCR (right) reactions. Total dark area increases over the course of amplification, corresponding to an increase in aggregation inhibition.

FIG. 27. Determination of starting template concentration and threshold time.

FIG. 28. Qualitative determination of human vs. non-human DNA samples.

FIG. 29. Product inhibited bead aggregation (PIA) where amplification product coats magnetic beads and full length DNA no longer induces aggregation.

FIG. 30. LAMP/PCR and PIA.

FIG. 31. Increasing LAMP product correlates with increasing inhibition.

FIG. 32. PIA controls.

FIG. 33. Time course for aggregation inhibition.

FIG. 34. Time course for aggregation inhibition during DNA amplification.

FIG. 35. Bar graph of aggregation inhibition by PCR product.

FIG. 36. Increasing aggregation versus cycle number.

FIG. 37. Use of PIA to detect different pathogens.

FIG. 38. Polyester toner disc including a plurality of devices according to one examples.

FIG. 39. Enlargement of polyester toner device that can be used with LAMP.

FIG. 40. Five layer chip formed of polyester with toner as adhesive including enlargement of single device with inlets/air vents and PiBA chamber.

FIG. 41. Serpentine channels for mixing on chip.

FIG. 42. Amplification profiles.

FIG. 43. Assay results using a rotating magnet(s) and spinning platform.

FIG. 44. Device with rotating magnet(s) and spinning platform.

FIG. 45. Lid mounted Android in 3D-printed case.

FIG. 46. Cell phone detection process.

FIG. 47. Image of results shown on cell phone.

FIG. 48. C. difficile detection of tcdB gene.

FIG. 49. Images of aggregation within PiBA chamber of the device of FIG. 40.

FIG. 50. Bar graph of percent difference in aggregation for various strains of C. difficile.

FIG. 51. Bar graph of percent difference in aggregation for various strains of C. difficile.

FIG. 52. Traditional assay.

FIG. 53. Cell phone detection.

FIG. 54A-54D. Electropherograms displaying specific amplification only to the targeted fluid of interest: Tris-EDTA buffer (Lane 1; negative control); blood (lane 2); vaginal fluid (lane 3); semen (lane 4); and saliva (lane 5).

FIG. 55. An image of the PMMA imaging booth used for 96-well plate imaging of LAMP samples.

FIG. 56. Cell phone image of 96-well plate containing mock LAMP samples that has been converted to the hue channel via Imagej. The rows are labeled according to the targeted body fluid and all contain a reference negative control on-board.

FIG. 57. Detailed sample workflow for preliminary body fluid identification of blood, vaginal fluid, saliva, and semen prior to obtaining a DNA profile. To identify body fluids, up to 23 unknown samples are isothermally amplified in fluid-specific primer assays for 30 minutes. A colorimetric change from purple to blue will occur only if samples contain the targeted body fluid. An optical image via a cell phone is taken post amplification and analyzed via hue. Tagging only amplified samples (hue<150) allows for accurate prediction of the body fluids contained in all samples simultaneously.

FIG. 58. An illustration of a block diagram illustrating an example of a machine upon which one or more embodiments of can be implemented.

FIG. 59. LAMP primer sequences for multiplexed body fluid identification.

FIG. 60. Surface plots of the first row of the 96-well plate from panel with amplified LAMP samples highlighted.

FIG. 61. Bar graph of hue and positive or negative samples.

FIG. 62. Electrophoretic gel displaying positive and negative LAMP controls of each body fluid using identified mRNA markers for blood, saliva, semen, and vaginal fluid.

FIG. 63. Comparison of varying concentrations of DTT with the rate of amplification for semen and saliva samples.

FIG. 64. Comparison of DTT vs β-mercaptoethanol (β-me) as a reducing agent for the LAMP Assay

FIG. 65. Time monitoring of RT-LAMP amplifications from a whole blood (reference) sample on blue and black denim material.

FIG. 66. Time monitoring of RT-LAMP amplifications from dried blood stains on blue and black denim material.

FIG. 67. Blind body fluid sample.

FIG. 68. Picture of 96-well plate.

FIG. 69. Picture of 96-well plate with corresponding image analysis of azospermatic semen samples. Duplicate samples from three different donors were compared to a negative control (containing only TE buffer for sample) and a non-specific control (1 ng human genomic DNA).

FIG. 70. Amplification Map of Explored Primer Sequences.

FIGS. 71A-71D. Pictures of 96-well plate with 5, 10, and 15 μL LAMP volumes with corresponding surface plots for each LAMP volume.

### DETAILED DESCRIPTION

### Example 1

### DNA Lysis and Extraction on a Polyethylene Terephthalate (Pe) Rotationally-Driven Microdevice

In one embodiment, a device and method are provided for on-chip sample lysis and an on-chip DNA extraction. In one embodiment, both of these processes are performed on a Pe-toner (PeT) microfluidic device composed of commercial-off-the-shelf (COTS) products and fabricated using ‘print, cut, laminate’ technology. Briefly, commercial printer toner is printed on both sides of Pe sheets which acts as the adhesive between each layer of the device. The architecture of each layer is then cut with a CO₂ laser before aligning the layers together and thermally laminating the layers to melt the toner and bond the layers of Pe for an enclosed system.

### On-Chip Sample Lysis

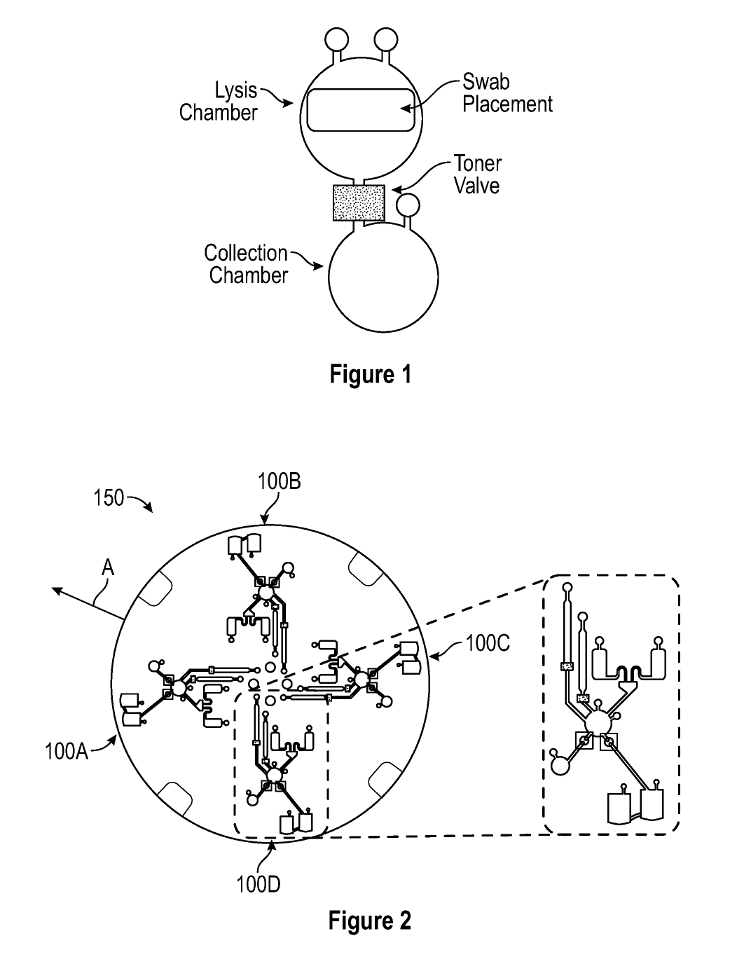

Sample lysis exploits the biological properties of cells in the presence of chaotropic solutions. Each device is 4 layers and has a square cutout in the top of the chamber, which accommodates the placement of a swab cutting that is to be tested (FIG. 1). A biocompatible adhesive sealed the chamber prior to filling with a chaotropic lysis buffer. Heating the chip on a heat block set at 56° C. allows the release of nuclear material. Initial experiments looked at parallel processing of a buccal swab cuttings: one placed in a chip for on-chip lysis and the other placed in a conventional tube lysis. Both samples, after the lysis step is completed, were brought through the same DNA processing steps (e.g., DNA extraction, quantitation, amplification, and detection) together, and, upon examining the STR profiles obtained from both samples, there were no differences in the STR profiles. This on-chip assay is therefore able to achieve successful on-chip lysis with a 66% reduction of incubation time and less than 8% of the original volume compared to conventional techniques. Thus, a contrifugally-driven DNA extraction microfluidic device formed of Pe and printer toner is provided.

### On-Chip DNA Extraction

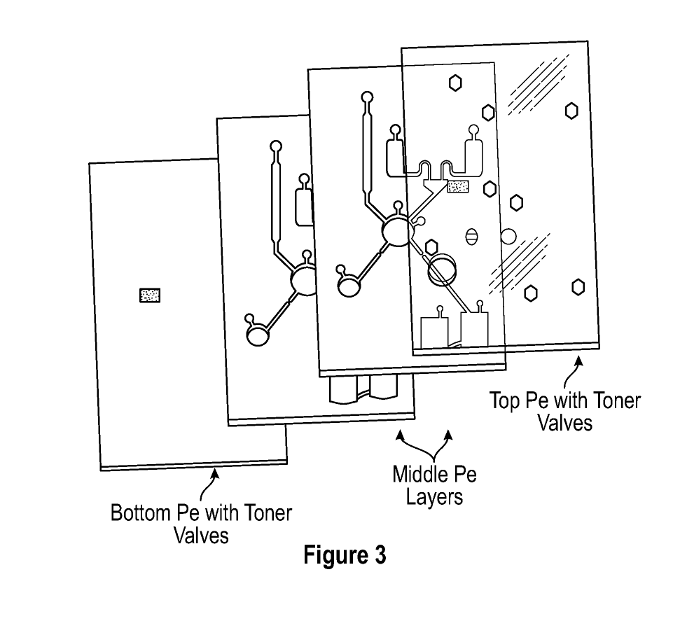

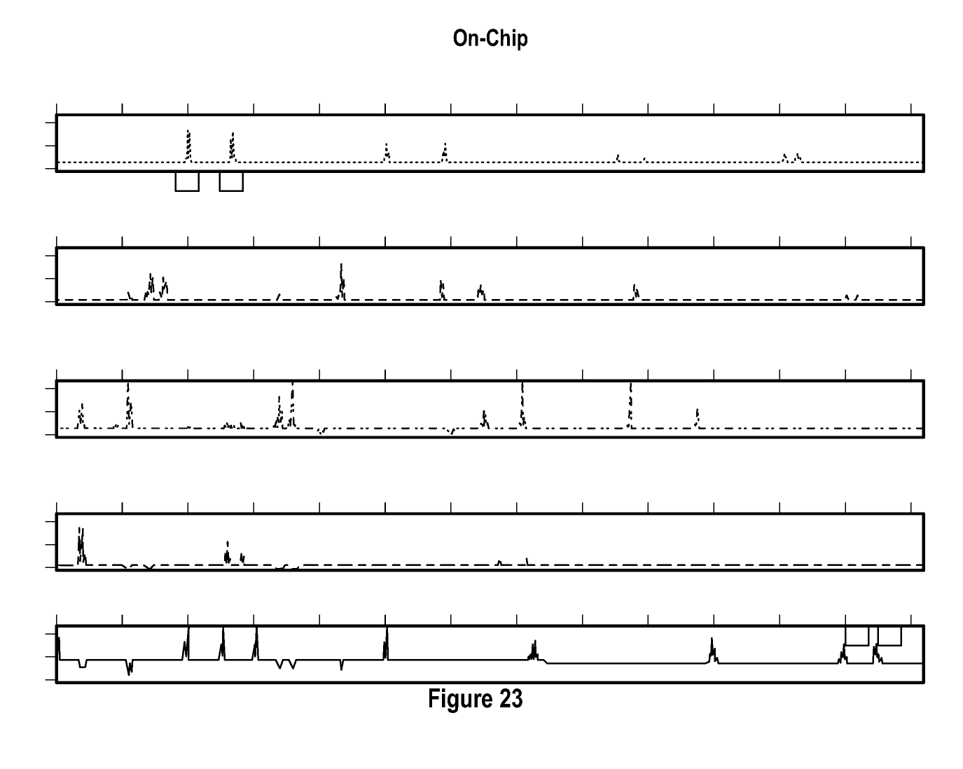

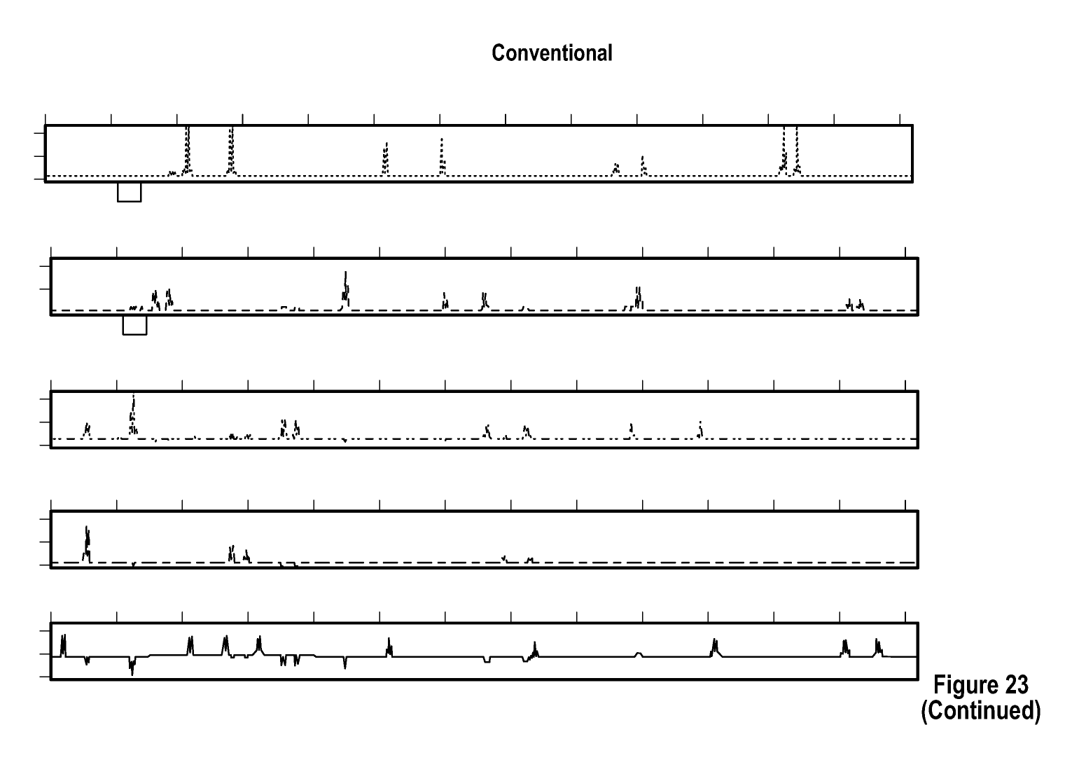

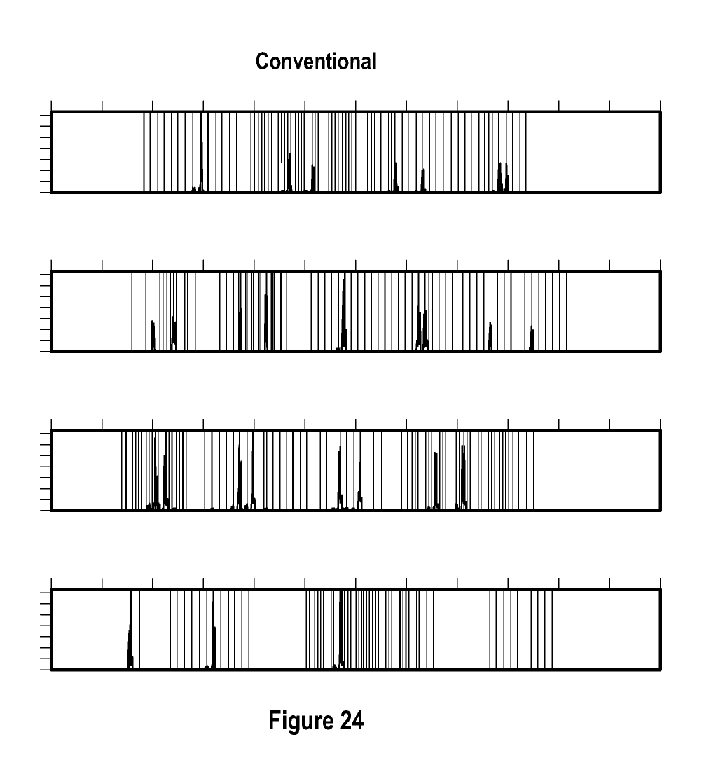

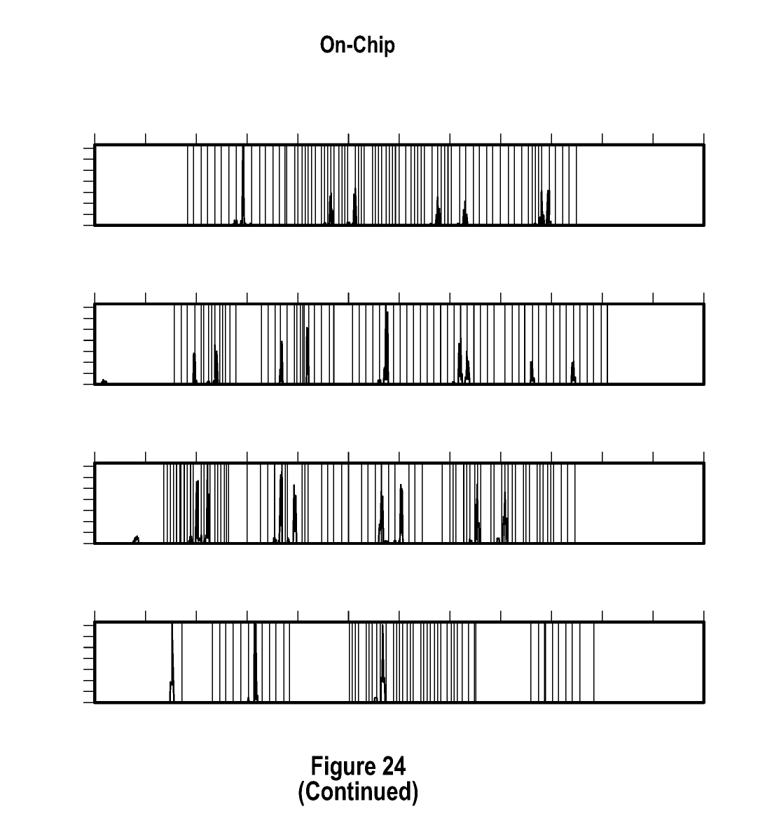

Dynamic solid-phase extraction (dSPE), or the purification of DNA by moving the magnetic silica particles through a static solution, was chosen for an extraction protocol, as it circumvents the reproducible packing issues of packed silica extractions. The device was moved to a centrifugal platform which avoids multiple manual pipetting and mixing steps. Fluid, therefore, can be pushed through the device from the center towards the outer edges by simply spinning the device. Two embodiments were based on the following dSPE protocol: (i) mixing the particles and DNA-containing sample in a chaotropic solution to bind the DNA to beads, (ii) washing the beads with an alcohol solution to solubilize any bound proteins and wash away contaminants, (iii) wash away residual alcohol with a low-salt buffer, (iv) release the DNA from the particles in a low-salt buffer, and (v) transfer the purified DNA to a separate collection chamber. In order to take the sample out of the device, a polymer sleeve, covering the top of a recovery chamber containing purified DNA, is punctured is extracted from the device using a syringe. The first device (FIGS. 2-3 and 5A and 5B) demonstrated successful DNA extraction (results shown in FIGS. 23 and 24), using a combination of passive and mechanical valves. The second device (FIG. 15) has enhanced dSPE microfluidic design automation with the conversion of all mechanical valves to passive valves.

a) Fluidic Control

Successful extractions require sequential addition of the reagents, which could only be achieved with fluidic valving. To keep a system fully automated, yet simple enough for portability, it was important to use passive valving wherever possible. Ouyang et al. identified the usefulness of ‘toner valves’, or patches of printer toner on the top and bottom of a channel that could produce a large surface tension to prevent the passing of a solution until the surface tension was overcome by centrifugal force. Although these valves were useful for holding a wash buffer and the elution buffer until needed in the extraction, they are not compatible with alcohol solutions. Furthermore, these ‘toner valves’ are only a one-time actuated valve which cannot keep waste solutions from entering the DNA collection chamber only meant for the purified DNA solution.

A combination of resistive elements was used to keep the alcohol from entering the main DNA chamber during the initial binding of DNA and the silica particles. First, the absence of air vents between the main chamber and the chambers holding the alcohol solution produced a backpressure when the main DNA chamber was filled. Furthermore, the chambers holding the alcohol were designed to hold an extra 0.5 μL of volume which allowed air to separate the alcohol from leaving the chamber. Together, these kept the alcohol in place and, only when the device spun, was air displaced, allowing the alcohol to flow freely through the device.

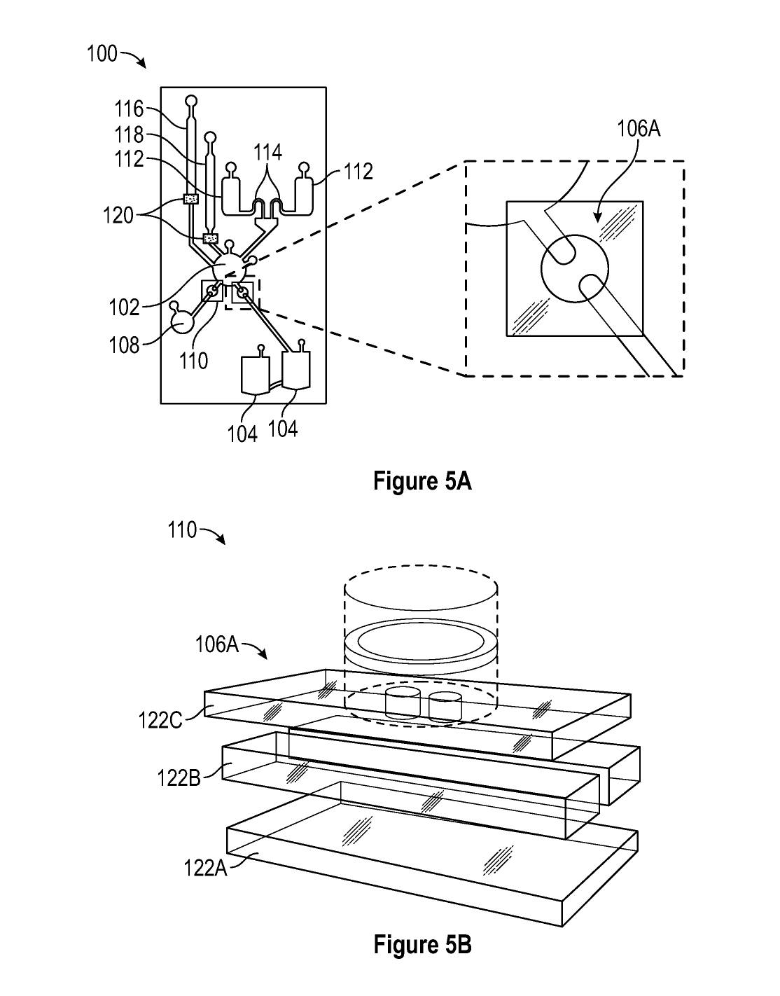

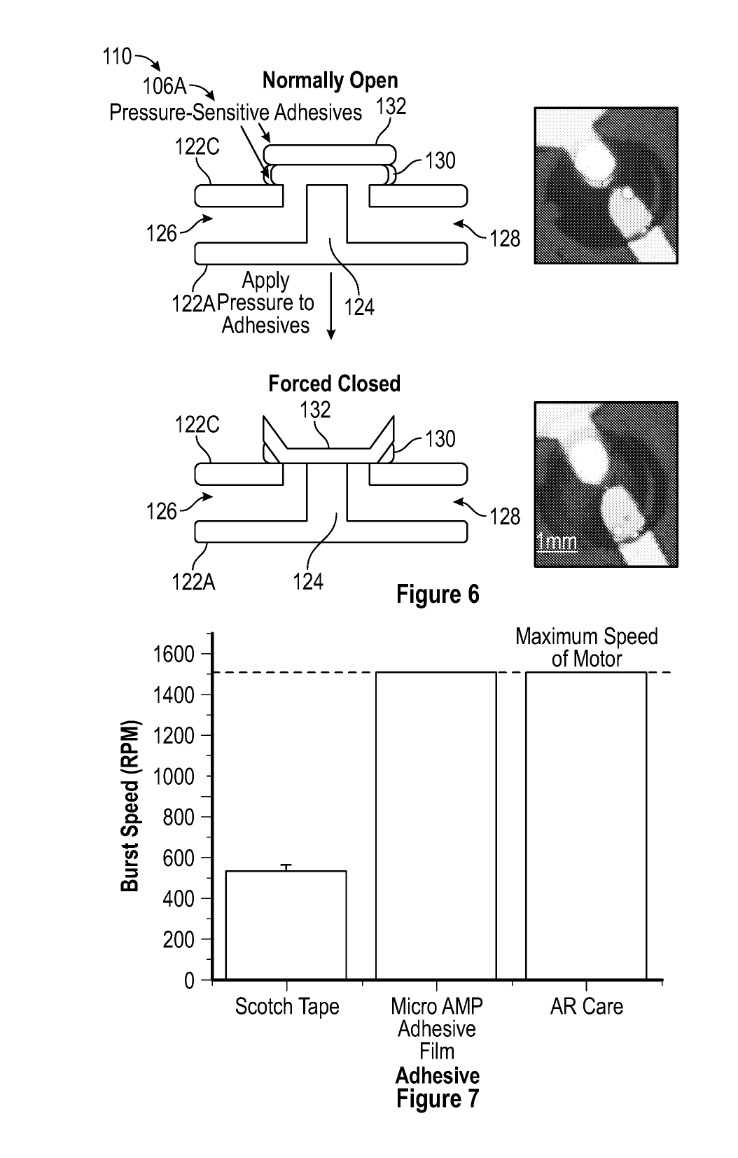

Two valves were used to steer solutions either to the waste chamber or the DNA collection chamber, both of which connect below the main DNA chamber: a siphon valve (FIG. 15) and a ‘tape valve’ also referred to as a stop valve herein (shown in FIGS. 2, 3, 5A-6). A novel ‘tape valve’, was used for controlled access to the DNA collection chamber. The ‘tape valve’ was fabricated in PeT in two or more layers, as shown in FIGS. 5A-6. The bottom layers were Pe material to make a valve seat and defined a physical barrier separating the incoming channel from the one coming out of the valve. The second layer was a double-sided adhesive that could stick to a piece of Scotch® tape which enclosed the device but also add height of the valve to lower the resistance for a solution to pass the physical barrier separating the channels.

According to the embodiment shown in FIG. 5A, a device 100 is shown. The device 100 can be configured to isolate nucleic acid through application of a centrifugal force thereto. The device 100 can include a mixing chamber 102, a waste chamber 104, a first stop valve 106A, a nucleic acid recovery chamber 108, a second stop valve 110, a first wash chamber 112, a second siphon valve(s) 114, an elution buffer chamber 116, a second wash chamber 118 and passive valves 120. According to another embodiment shown in FIG. 15, the first stop valve 106A has been replaced with a siphon valve 106B.

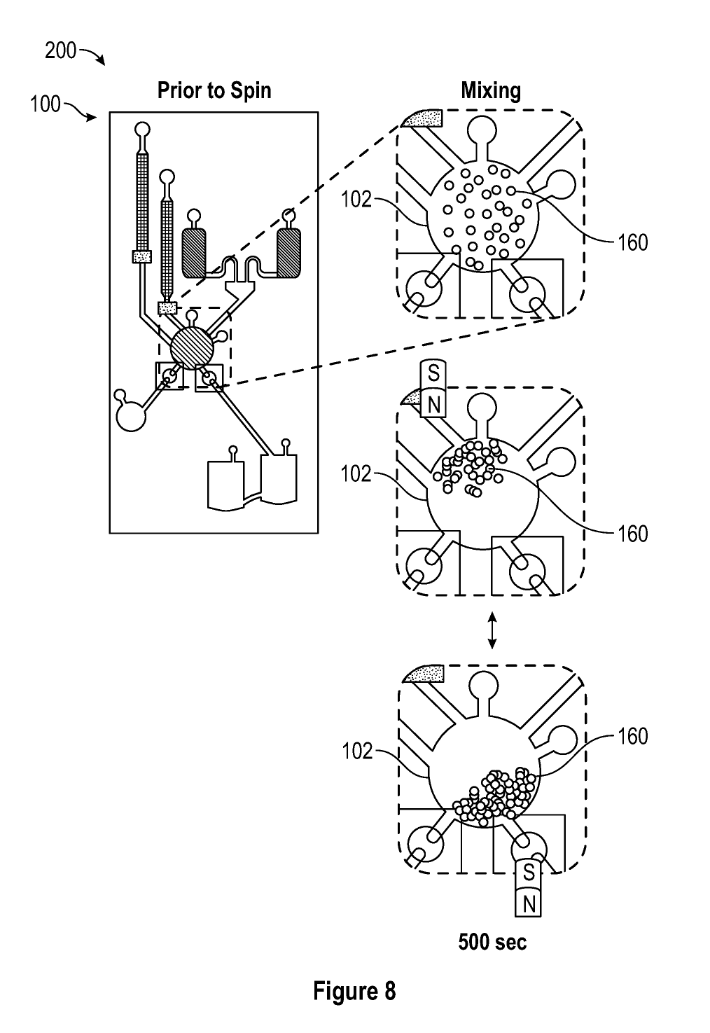

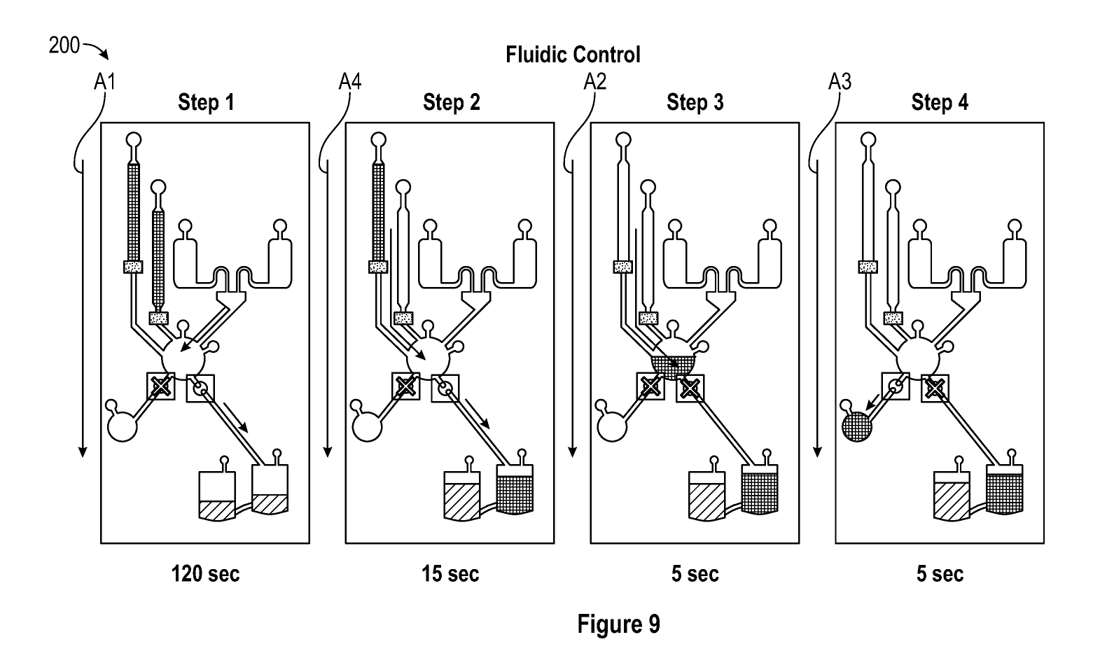

As shown in FIGS. 5A and 15, the mixing chamber 102 can selectively connected to one or more of a first wash buffer (shown in FIGS. 2 and 8 and housed in the first wash buffer chamber 112) and an elution buffer (shown in FIGS. 2 and 8 and housed in the elution buffer chamber 116). The mixing chamber can be configured to receive a sample therein as shown in FIGS. 2 and 8. The waste chamber 104 can be selectively connected to the mixing chamber 102 by one of the first stop valve 106A and the first siphon valve 106B. The first stop valve 106A can be configured to be forced open after the device is rotated at a first rotational speed with the first wash buffer disposed in the mixing chamber 102 to allow for passage of a waste from the mixing chamber 102 to the waste chamber 104 (process shown in FIG. 9 as step 1). The nucleic acid recovery chamber 108 can be selectively connected to the mixing chamber 102 by the second stop valve 110. The second stop valve 110 can be configured to be forced open after the device 100 is rotated at a second rotational speed with the elution buffer disposed in the mixing chamber 102 to allow for passage of the nucleic acid from the mixing chamber 102 to the nucleic acid recovery chamber 108 (process shown in FIG. 9 as step 4). The second rotational speed can differ from the first rotational speed.

According to some embodiments, the first stop valve 106A can be configured to be forced open as illustrated FIG. 6 at a first burst pressure to allow for passage of the waste from the mixing chamber 102 to the waste chamber 104 (process shown in FIG. 9 as step 1). The second stop valve 110 can be configured to be forced open (again as illustrated in FIG. 6) at a second burst pressure that differs from the first burst pressure to allow for passage of the nucleic acid from the mixing chamber 102 to the nucleic acid recovery chamber 104 (process shown in FIG. 9 as step 4) The second burst pressure can be lower than the first burst pressure.

The elution buffer chamber 116 can be configured to contain the elution buffer and can be selectively connected to the mixing chamber 102 by one of the passive valves 102 (e.g., a first hydrophobic valve). The first wash chamber 112 can be configured to contain the first wash buffer and can be selectively connected to the mixing chamber 102 by the second siphon valve 114. The second wash chamber 118 can be configured to contain the second wash buffer and can be selectively connected to the mixing chamber 102 by a second of the passive valves 120 (e.g., a second hydrophobic valve).

The elution buffer can comprise a chaotropic solution to bind the nucleic acid to beads. The first wash buffer can comprise an alcohol solution and the second wash buffer can comprise a low salt buffer. The first stop valve 106A and the second stop valve 110 can be formed of a plurality of polyethylene terephthalate layers 122A, 122B, 122C as shown in FIG. 5B including a first layer 122A (FIGS. 5B and 6) configured to define a valve seat 124 (FIG. 6) that comprises a physical barrier separating an incoming channel 126 (FIG. 6) from an outgoing channel 128 (FIG. 6) of the valve 106A. The plurality of polyethylene terephthalate layers 122A, 122B, and/ 122C can include a second layer 122B or 122C configured to form at least a portion of the incoming channel 126 (FIG. 6) and the outgoing channel 128 (FIG. 6) of the valve 106A. As shown in FIG. 6, in some cases the valve 106A can also include a double-sided adhesive 130 configured to adhere to the second layer 122B or 122C. The valve 106A can further include a second adhesive 132 comprising a pressure-sensitive adhesive that is couple to the double-sided adhesive 130.

According to the embodiment of FIG. 15, the first siphon valve 106B can be configured to allow access to the waste within the waste chamber 104 after an initial binding, and during an alcohol wash, and is further configured to prevent the elution buffer from entering the waste chamber 104.

The device 100 can be part of a disc 150 as illustrated in FIG. 2. Thus, the device 100 referred to herein can actually comprise a plurality of devices 100A, 100B, 100C and 100D that are circumferentially distributed around the disc 150 such that the centrifugal force (indicated with arrow A) can be applied on the plurality of devices 100A, 100B, 100C and 100D simultaneously.

In one embodiment of a dSPE microfluidic device as shown FIGS. 2, 3 and 5A and 5B, two ‘tape valves’ were used to separate both the waste chamber and the DNA chamber as well as the DNA collection chamber from the main DNA chamber. At the beginning of an extraction, the ‘tape valve’ between the DNA collection chamber and the main chamber would be closed while the ‘tape valve’ to the waste chamber was in the open position. This allows all solutions to be driven down to the waste chambers. Once the washing steps were completed and the elution step was to be next, a blunt object was used to close the ‘tape valve’ that separated the waste chamber from the main DNA chamber by applying pressure to the adhesive that sits directly above the physical barrier of the valve (FIG. 6). At this point, both ‘tape valves’ are closed, only allowing fluids to come to the main DNA chamber and no further. The elution solution, once released from the toner valve, comes down to the main chamber and is ready to mix with the magnetic particles to release the DNA. In order to transfer the elution solution to the DNA collection chamber and not to the waste, the width of the physical barriers of the ‘tape valves’ had to be different. The physical barrier separating the incoming from the outgoing channel was thinner for the ‘tape valve’ directing fluid to the DNA collection chamber, therefore, this ‘tape valve’ had a lower burst pressure and would be forced open first (FIGS. 6 and 9). A schematic of this process is shown in FIGS. 8 and 9.

The process of FIGS. 8 and 9 can comprise a method 200 of extracting nucleic acid using a device (e.g. device 100) constructed of plurality of polyethylene terephthalate layers as previously described. The method 200 can include loading a sample into the mixing chamber 102 formed by one or more of the plurality of polyethylene terephthalate layers. The mixing chamber 102 can be connected to the waste chamber 104 by one of a first stop valve 106A and a first siphon valve 106B (FIG. 15). The mixing chamber 102 can be selectively connected to a nucleic acid recovery chamber 108 by the second stop valve 110 as previously described in reference to FIGS. 5A and 15. The method 200 can include rotating the device at a first rotational speed (indicated by arrow A1) to release a first wash (shown by arrow in step 1) into the mixing chamber 102 and subsequently through the first stop valve 106A or the first siphon valve into the waste chamber 104 (also illustrated in step 1). As shown in step 2 of FIG. 9, a second wash buffer can be released by rotating the device at a fourth rotational speed (indicated by arrow A4) to release a second wash (shown by arrow in step 2) into the mixing chamber 102 and subsequently through the first stop valve 106A or the first siphon valve into the waste chamber 104. The method 200 can further include rotating the device at a second rotational speed (indicated by arrow A2) in step 3 to release an elution buffer (shown by arrow in step 3) to the mixing chamber 102 to mix with the sample and magnetic particles (shown as items 160 in FIG. 8) to release the nucleic acid. At step 4, the method 200 can rotate the device 100 at a third rotational speed (indicated by arrow A3 in step 4) to pass the nucleic acid through the second stop valve 110 into the nucleic acid recovery chamber 108. In some cases, rotating the device 100 at the third rotational speed to pass the nucleic acid through the second stop valve can include forcing the second stop valve 110 open by exceeding a burst pressure thereof. In some cases (as is described in reference to FIG. 9), the first rotational speed, the second rotational speed, the third rotational speed, and the fourth rotational speed all differ

In some embodiments of the method, sequent to rotating the device 100 to release the first wash, the method 200 can open the first stop valve to the waste chamber. In some embodiments, sequent to rotating the device 100 to release an elution buffer to the mixing chamber, the method 200 can close the first stop valve to the waste chamber.

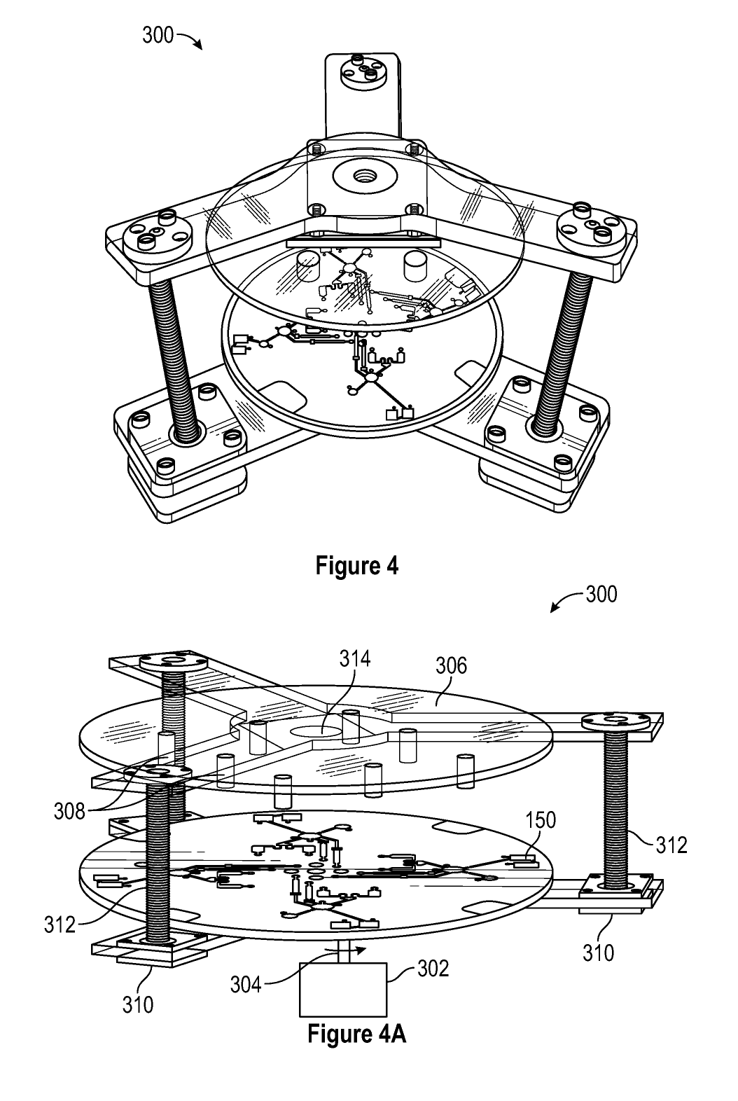

As shown in the embodiment of FIG. 8, the method 200 can further include applying a magnetic force (F) on the magnetic particles 160 within the mixing chamber 102. The device 100 is configured to be rotated both clockwise and counter-clockwise (shown in the two images with the magnet of FIG. 8) to change a direction of the magnetic force (F) on the particles 160 to mix the magnetic particles 160 within the mixing chamber 102. The method 200 can vary the intensity of the magnetic force on the magnetic particles within the mixing chamber by bringing the device 100 closer to the magnet as is further described in reference to the embodiment of FIGS. 4 and 4A. The manual closing of the tape valve compromised the automation of the device, therefore, improvements in the valving were done in the second embodiment. One of these improvements was to replace the ‘tape valve’ between the waste and the main DNA chamber with the siphon valve 106B of FIG. 15. A siphon valve was used to allow access to the waste reagents after initial binding, during the alcohol wash, and to eliminate residual alcohol, yet produce enough resistance such that when waste chambers would be full after all washing steps were completed, it would prevent the elution buffer from going to the waste. This allowed the elution buffer to be kept in the main DNA chamber. The extraction began with the ‘tape valve’ in the closed position, forcing all solutions to pass through to the waste chambers. Only when the device was spun in excess of the valve's burst pressure of 550 revolutions per minute (RPM) was the valve forced open. After the DNA was released from the particles, the device was spun at a high acceleration to 1000 RPM so the ‘tape valve’ was forced open before the solution could go through the siphon valve.

Thus, a device is provided having a first ‘tape valve’ structure fabricated and demonstrated on a PeT microfluidic device; as well as the use of passive valving techniques to contain alcohol solutions on a PeT microfluidic device.

FIGS. 4 and 4A show an apparatus 300 that can be used to rotate the discs 150 and devices 100 previously described. The apparatus 300 can be configured to carry the centrifugal microfluidic device 100 to prepare a sample for analysis. The apparatus can be configured to apply centrifugal force to the disc 150 as previous described. The disc 150 can be mounted to the apparatus 300 as shown in FIG. 4A. As previously discussed, the disc 150 can be constructed of plurality of polyethylene terephthalate layers configured to form a plurality of devices 100. As shown in FIG. 4A, the apparatus 300 can further include a first motor 302 configured to rotate the disc 150 via a shaft 304, for example. The apparatus 300 can also include a second device 306 disposed a distance apart from the disc 150. The second device 306 can have one or more magnets 308 mounted thereto. The one or more magnets 308 are configured to exert a magnetic force on particles within the mixing chamber 102 as previously described and illustrated in reference to FIG. 8. The disc 150 can be configured to be rotated both clockwise and counter-clockwise by the apparatus 100 relative to the one or more magnets 308 and the second device 306 to change a direction of the magnetic force on the particles to mix the particles within the mixing chamber (as illustrated in FIG. 8). The apparatus 300 can have a second motor 310 (e.g. a stepper motor) configured to adjust the distance apart the disc 150 is disposed from the second device 306. Such adjustment can be via height adjustment screws 312, for example, The second device 306 can be shafted or otherwise connected to a third motor 314 (e.g. a spin motor) so that the magnets 308 on the second device 306 can be rotated to be repositioned as desired in some embodiments.

b) Magnetic Induced Mixing

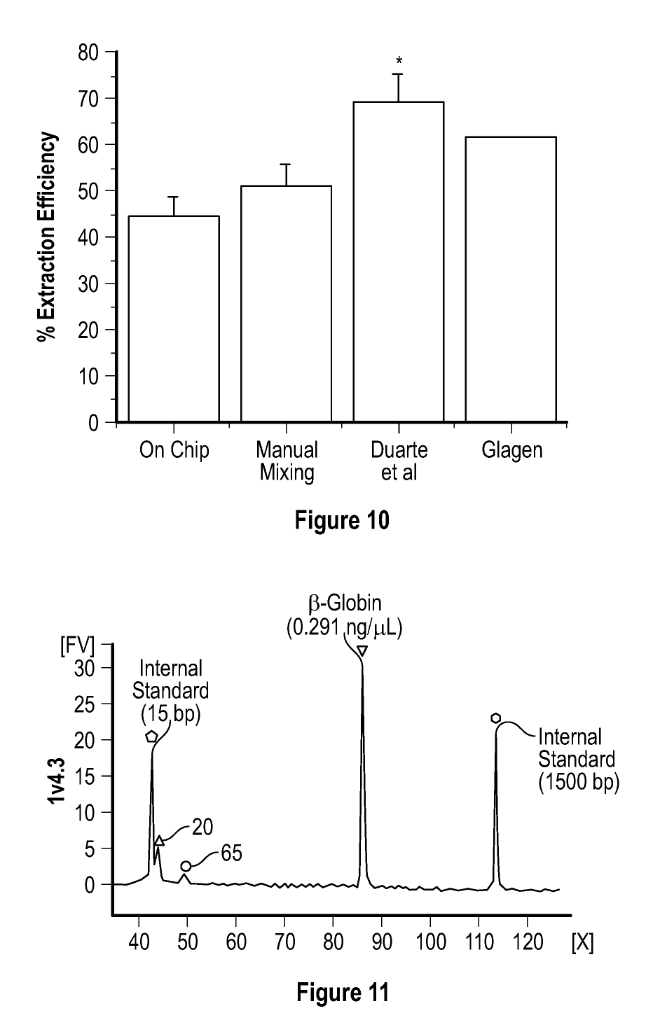

Unlike all other extraction centrifugal platforms, the magnetic field remained static and the sample disc containing the dSPE reagents were moved bidirectionally about 43°. Two magnets were placed such that when the sample disc moved 43° counter-clockwise, the beads are brought to the upper left corner of the chamber. The disc is then brought 43° clockwise and comes in contact with a second magnet that brings the particles to the lower right corner of the chamber. The disc would then repeat this cycle, sweeping the beads to opposing corners of the chamber for full mixing (Figures as illustrated and described in reference to FIGS. 8-9). Validation of this mixing strategy was confirmed using fluorospectrometry for quantitating the presence of DNA followed by successful amplification of β-globin (FIGS. 10 and 11).

Therefore, magnetic mixing may be conducted with a microfluidic device moving around stationary magnets.

c) STR Amplification

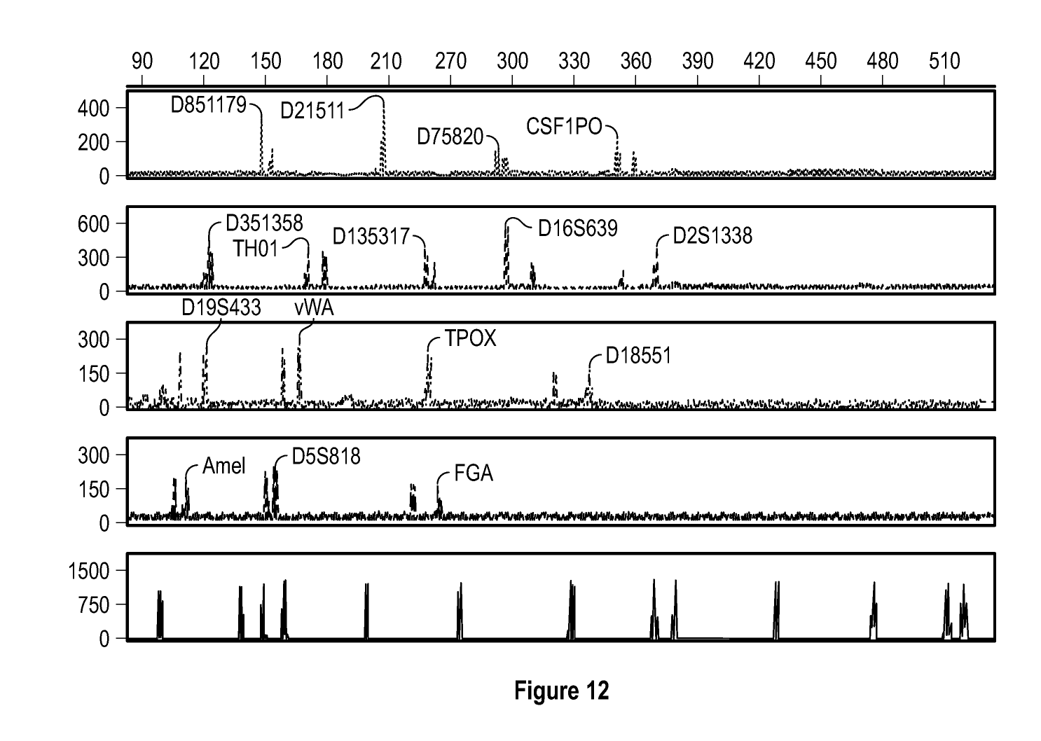

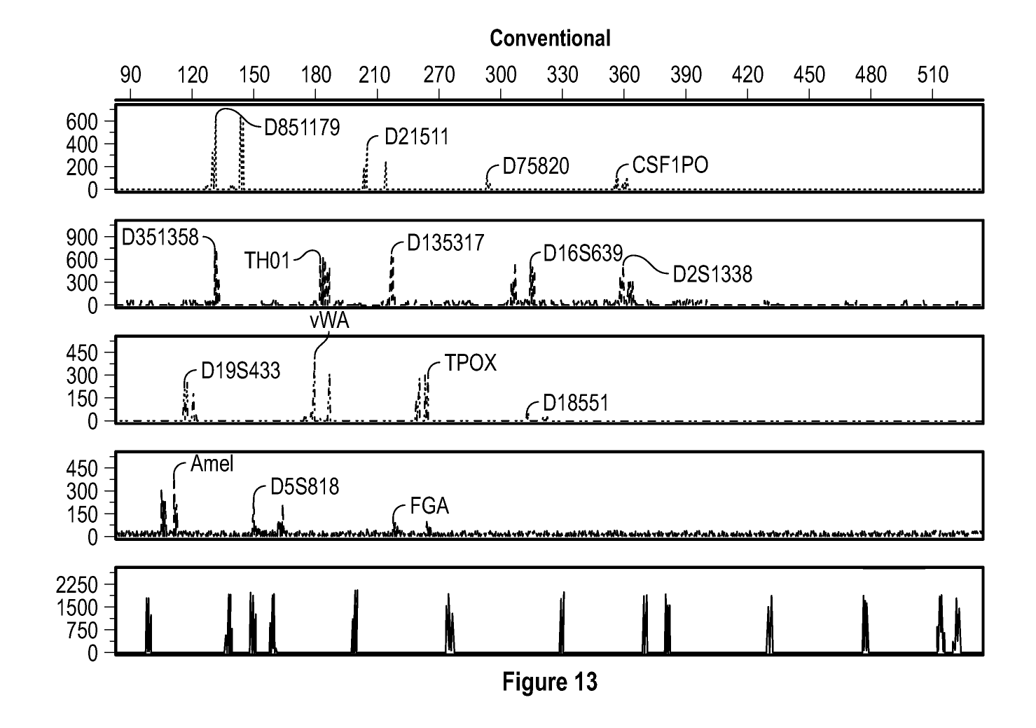

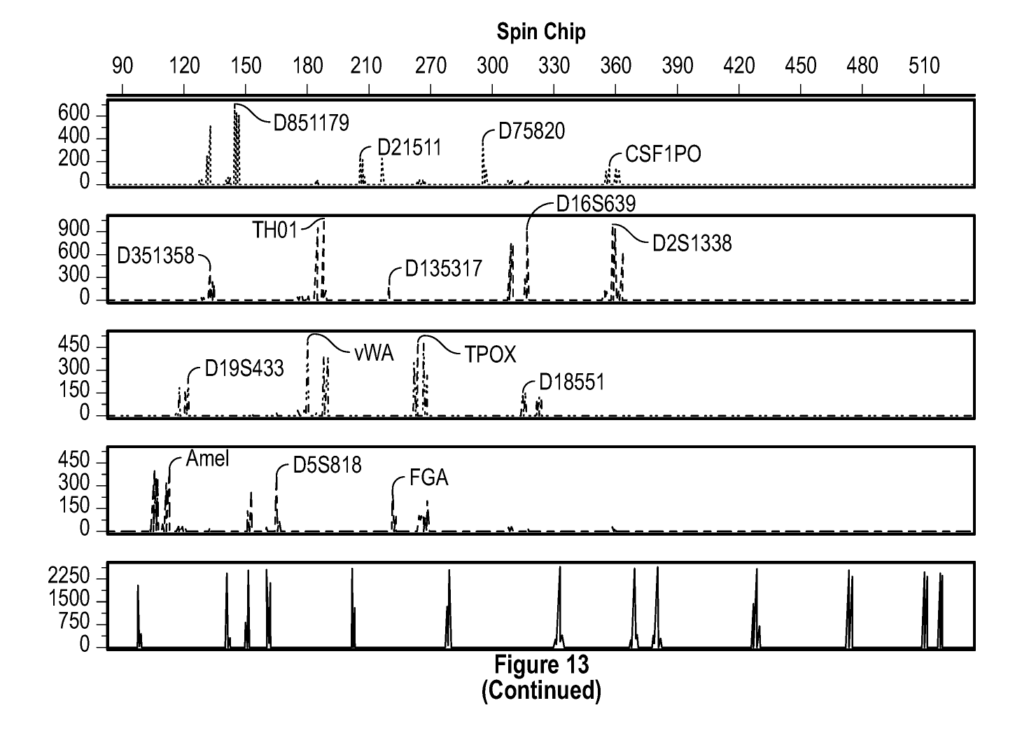

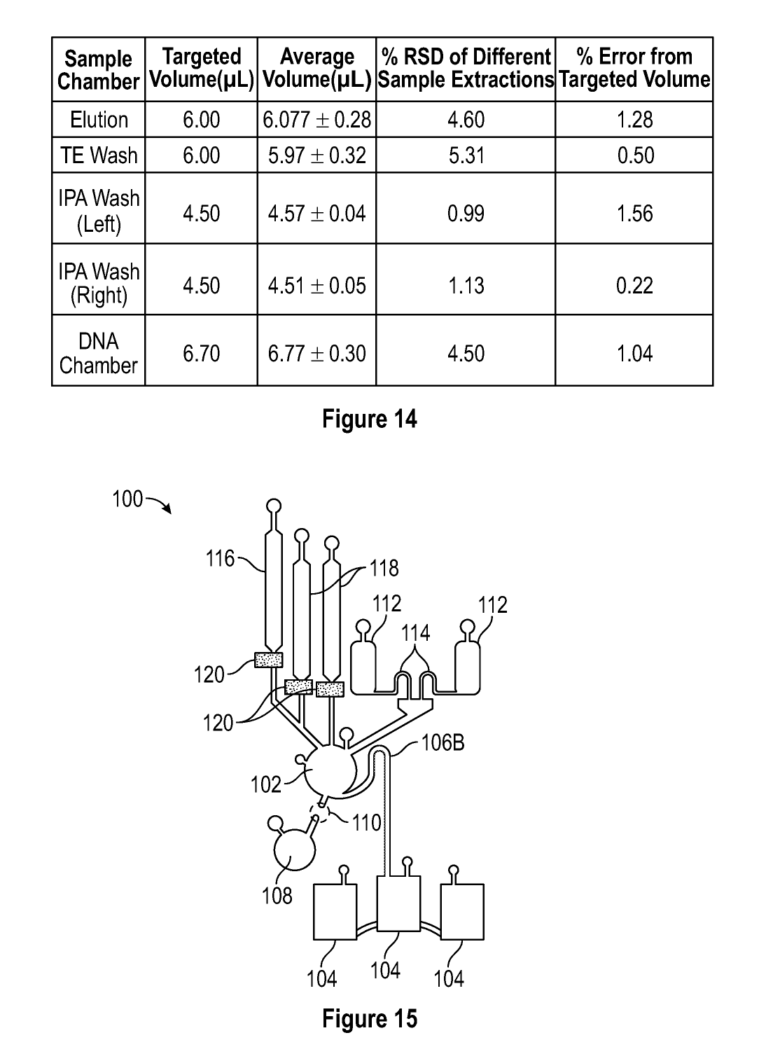

DNA collected from a centrifugally-driven DNA extraction microdevice produced full STR profiles that were in 100% agreement with conventional benchtop processes (FIGS. 10-13). Chamber volumes and deviations are shown in FIG. 14. The profile quality (e.g., peak height and peak balance) was compared and there were no significant differences. Therefore, the PeT dSPE device can be used for reliable DNA extraction of forensically-relevant samples. Figures

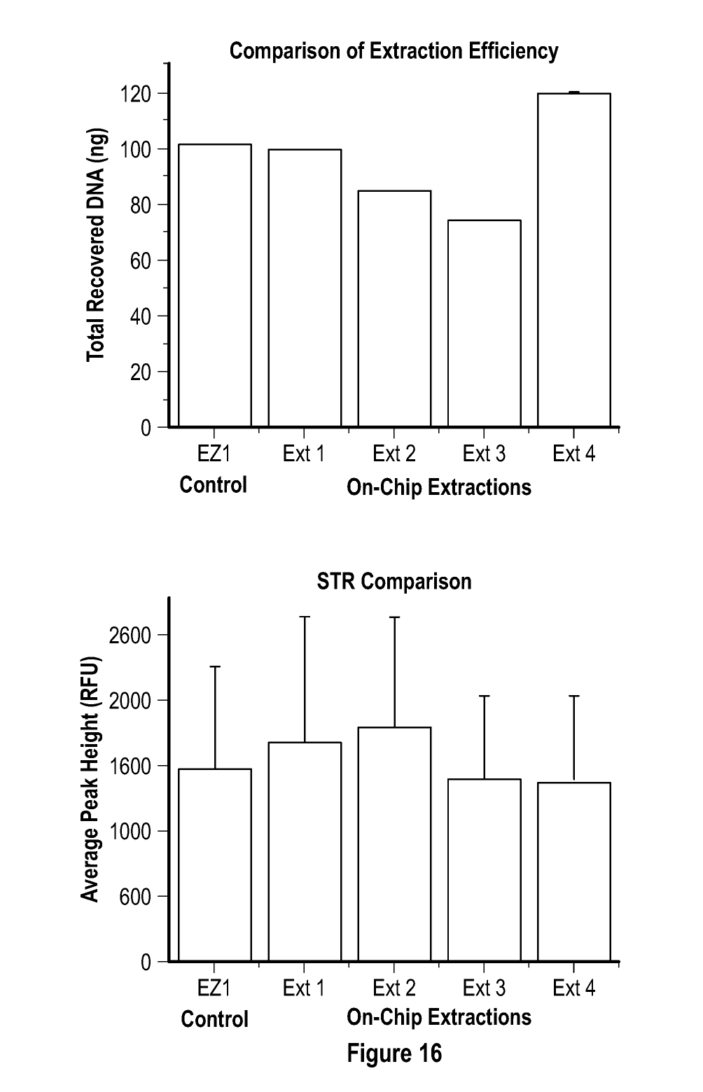

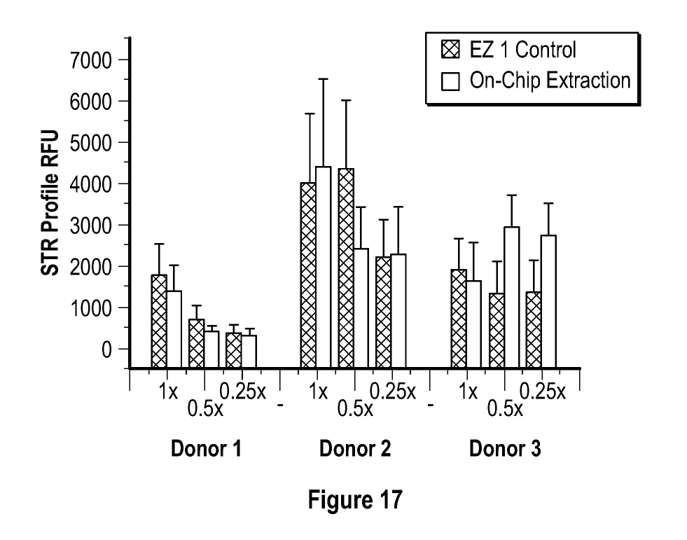

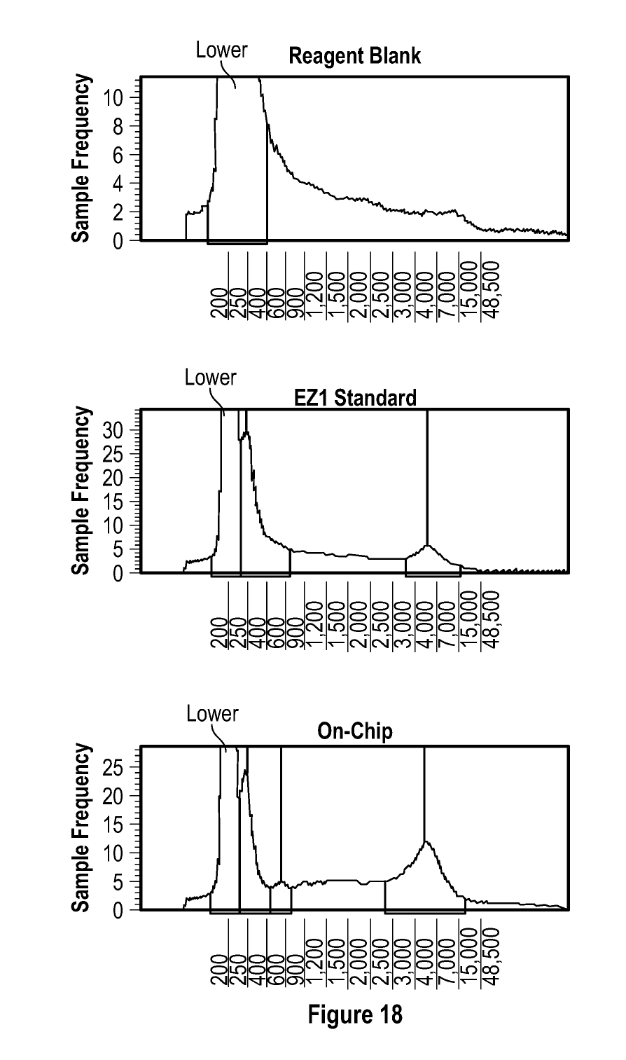

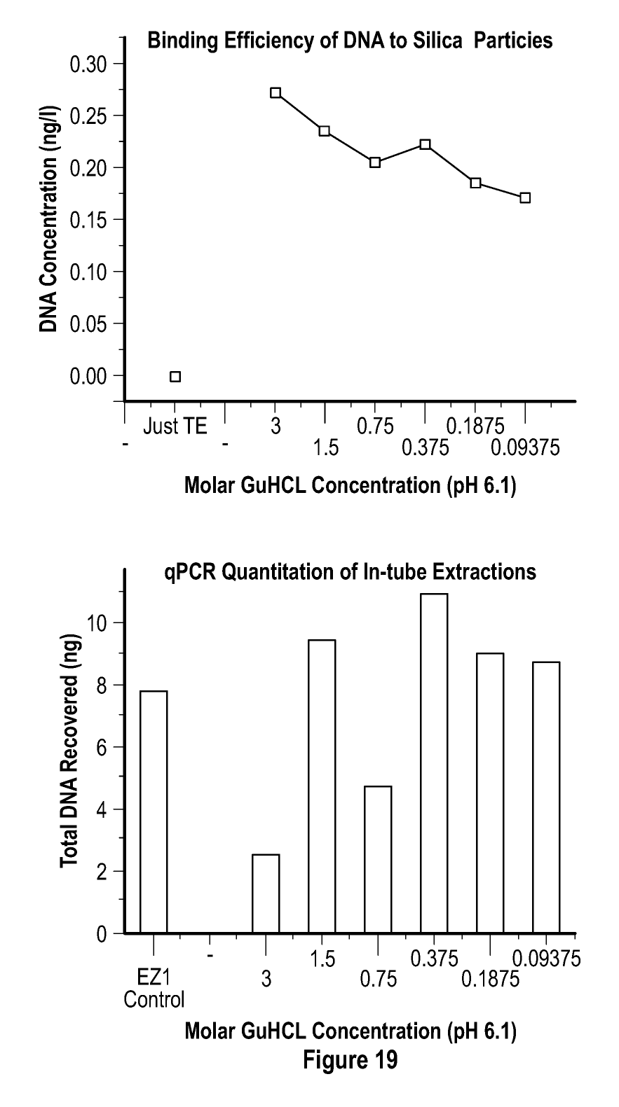

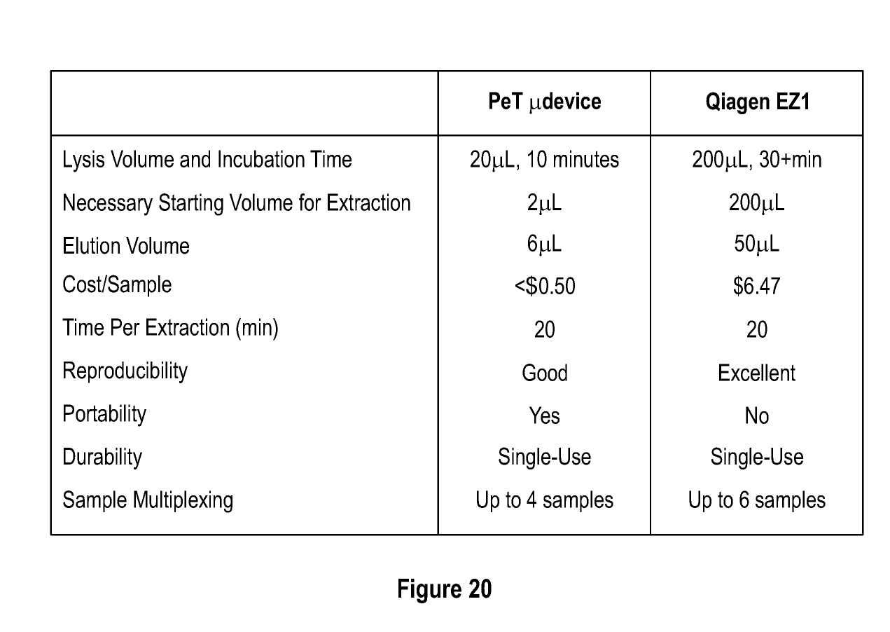

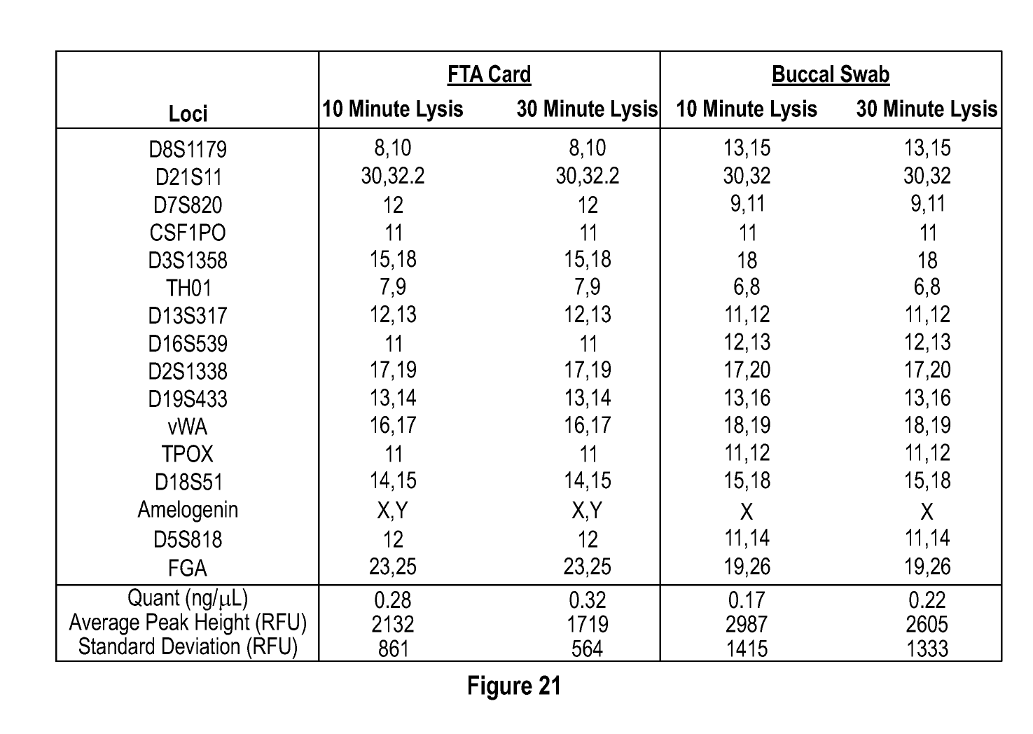

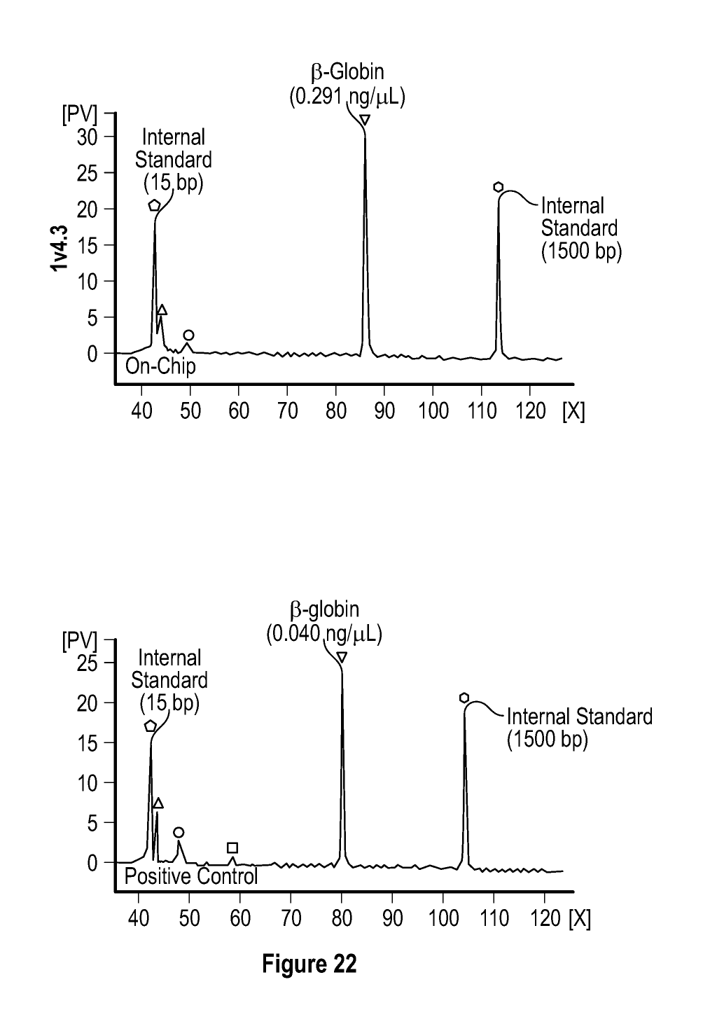

Another embodiment for DNA extraction and device results are shown in FIGS. 15-24. Workflow was also validated with STR profiles from blood and buccal swab samples (FIGS. 23-24).

Thus, a multiplexed amplification was demonstrated from an automated DNA extraction device, and DNA from a centrifugally-driven DNA extraction microfluidic device was shown to be compatible with multiplexed amplification.

In one embodiment of the device (and related method), very small volumes are used for DNA extraction; e.g., 0.6 μL of whole blood (2 μL lysed sample) or buccal cutting to obtain full STR profiles and/or amplification of β-globin. Therefore, DNA extraction has been demonstrated on a centrifugally-driven polyethylene terephthalate material and toner device. A microdevice can operate using all passive valving and the magnetic mixing is based on the device moving around static magnets.

### Example 2

### System and Method for Product-Inhibited Aggregation (PIA)

A current standard for many clinical diagnostic applications requiring nucleic acid amplification is real-time, quantitative polymerase chain reaction (qPCR). This technique generally utilizes either fluorescent dyes that intercalate into the DNA duplex as it is being produced, or fluorophores that are attached to sequence-specific oligonucleotide probes and hybridize to complementary sequences of messenger RNA (mRNA). A measure of fluorescence intensity allows for real-time monitoring of the reaction; however, this requires complex instrumentation and expensive equipment.

An alternative method for nucleic acid amplification, first published by Notomi and colleagues in 2000, is referred to as loop-mediated isothermal amplification (LAMP). This technique utilizes four specially designed primers that recognize six distinct sequences on target DNA/RNA. LAMP is capable of amplifying nucleic acids with high specificity, efficiency, and rapidity under isothermal conditions. This reaction produces magnesium pyrophosphate as a byproduct, and thus, real-time monitoring can either be accomplished via fluorescence intensity (similar to qPCR), or via changes in turbidity. As with qPCR, current methods for real-time monitoring of LAMP reactions require costly instrumentation.

Long strands of generic, human genomic DNA (hgDNA, 100 Kb) and specific amplification product are introduced into each well of a microdevice simultaneously in the presence of 6M guanidine HCl and silica-coated magnetic beads. Under the influence of a rotating magnetic field and gentle vortexing, the long DNA strands will interact with the surface of the magnetic beads and induce aggregation; however, when amplification product (LAMP, PCR) is present the aggregation is inhibited. This is likely attributable to short amplicon fragments binding to the surface of the magnetic beads, thereby restricting access of the longer DNA strands. Utilizing an image analysis algorithm, the ‘total dark area’ of each well is computed as a product of the number of dark vs. light pixels present.

### Exemplary Results

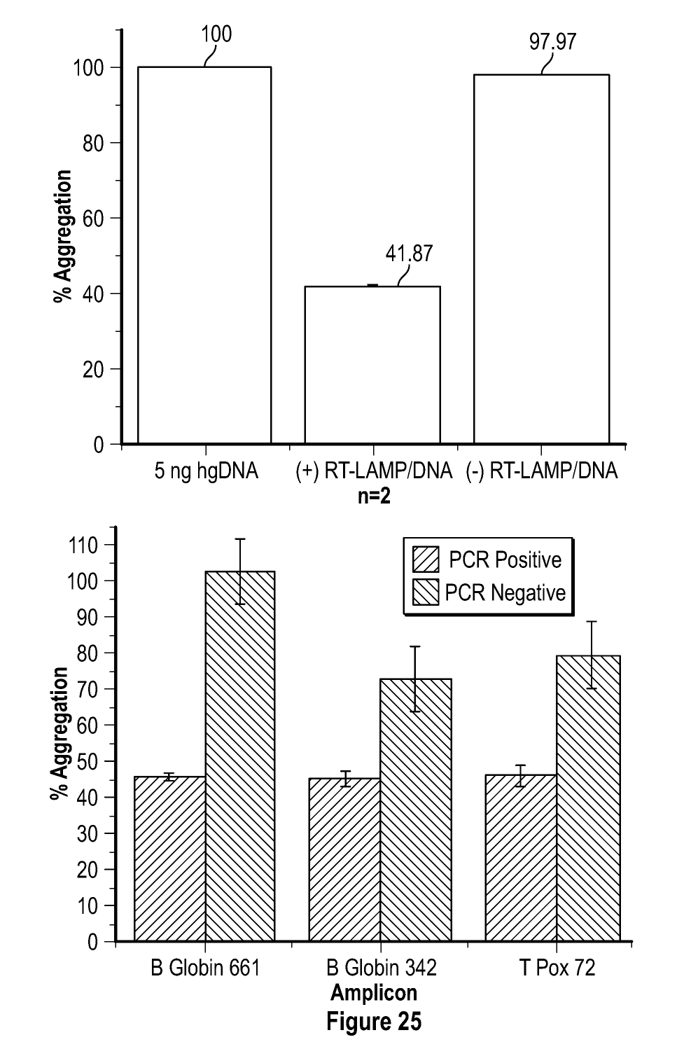

In a qualitative manner, the presence or absence of LAMP/PCR product can be determined by looking at the extent of aggregation inhibition (FIG. 25). When the amplification is successful, a specific amplicon is produced, and aggregation is inhibited. If the amplification is unsuccessful, no amplicon is produced, and the long DNA strands are able to access the surface of the magnetic beads and induced aggregation.

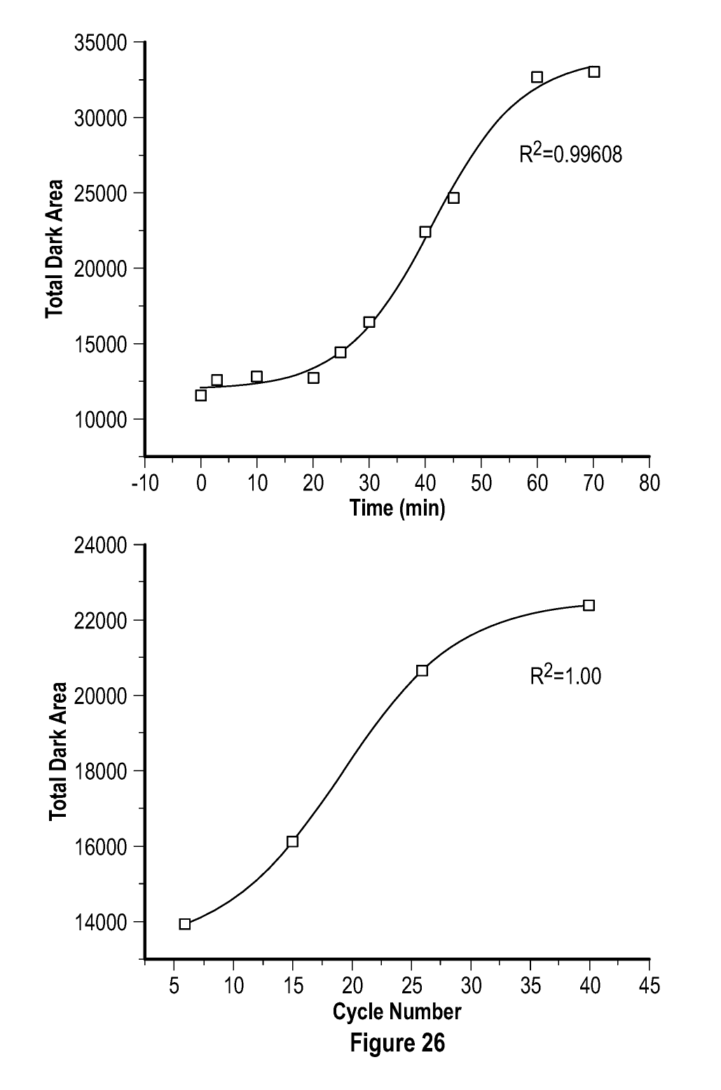

There may be a concentration effect on aggregation inhibition. Rift Valley Fever Virus was amplified using RT-LAMP and the progress of the reaction was monitored in real-time. Samples were assayed using PIA over the time course of the reaction. In a similar fashion, β Globin was amplified using PCR and monitored in a real-time fashion utilizing the PIA assay. FIG. 26 shows an increase in the total dark area as the amplification reactions proceed (both RT-LAMP and PCR), suggesting an increase in aggregation inhibition. These curves show similar features to real-time PCR/LAMP reactions, including baseline, exponential, linear, and plateau regions.

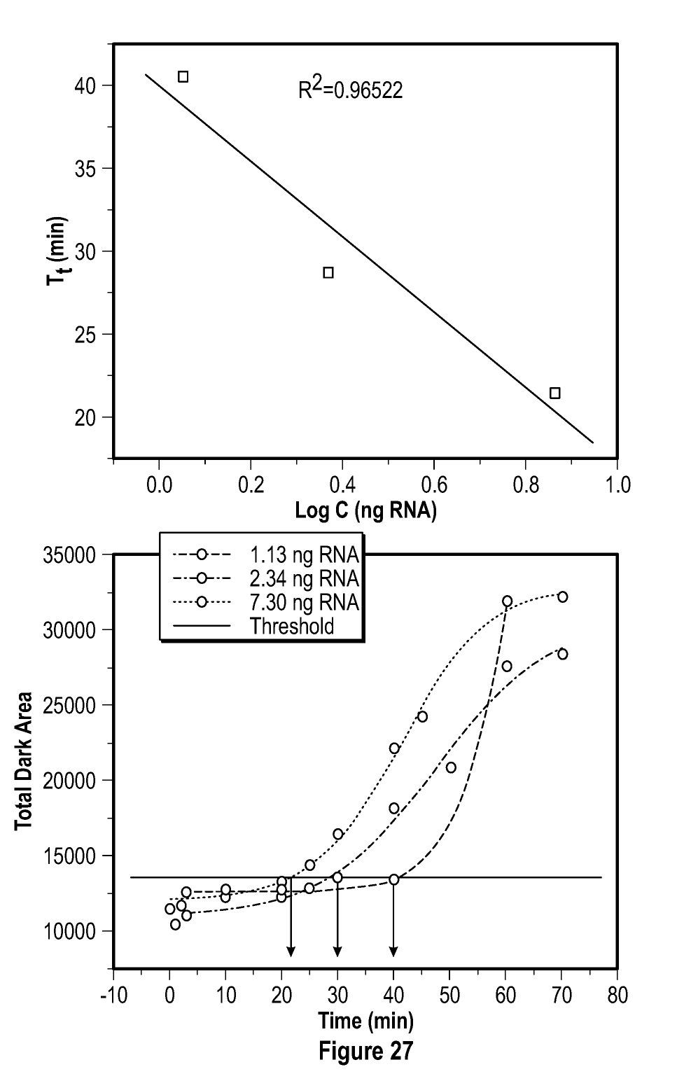

Data was collected from three different starting template concentrations for RVFV RT-LAMP (FIG. 27). A threshold was set at 15% of the average baseline for the three amplifications, and threshold times (Tₜ) were calculated for each. FIG. 27 also shows the plot of Log C (starting template concentration of RNA) versus threshold time. From this plot, it is possible to quantify the starting template concentration of the amplification.

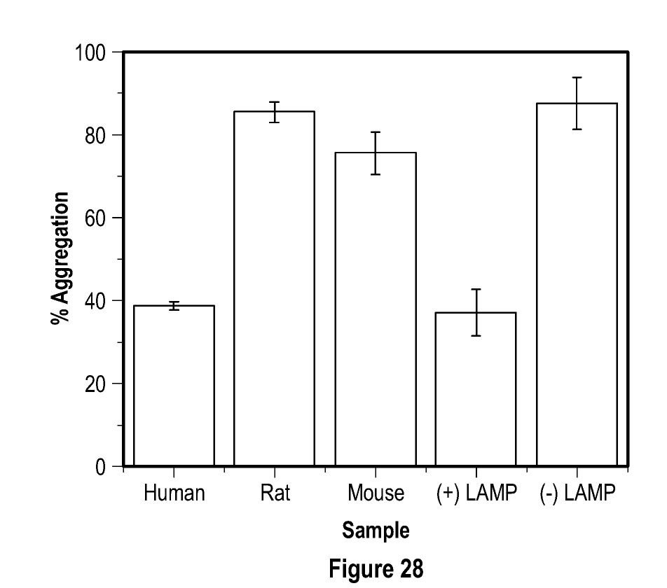

Using a LAMP assay specific for the T Pox gene, PIA has been preliminarily shown to have promise in identifying an unknown sample as containing human vs. non-human DNA. FIG. 28 shows qualitative data to differentiate a human DNA sample from other animal samples. T Pox will specifically amplify only in human samples, thus aggregation inhibition in the PIA response signifies human DNA.

Thus, a method and system for qualitative detection, quantification, and real-time monitoring of nucleic acid amplification products using magnetic bead aggregation inhibition are provided. The simultaneous introduction of long DNA strands and specific amplification product (e.g., generated either with PCR or LAMP) to a microwell containing silica-coated magnetic beads allows for the extent of aggregation inhibition to be measured using an image analysis algorithm.

In the presence of specific amplification product, aggregation is inhibited in a quantifiable manner and represented as low % aggregation (i.e. high total dark area). This method and system offers, among other things, a simple and inexpensive alternative to standard methods for real-time monitoring of LAMP/PCR, as well as qualitative pathogen detection.

Therefore, aggregation inhibition is utilized to quantitate short fragments of DNA generated from amplification reactions. This can be useful for simple, inexpensive, real-time monitoring of these processes without the requirement for flourophores or labels (“label-free”). In one embodiment of the method and system, only a camera and a simple computer algorithm may be used for real-time amplification reaction monitoring. The specificity of the associated technique is built into the amplification, and allows for the differentiation between specific (successful) and non-specific (unsuccessful) amplifications. In one embodiment, the technique is integrated into a microfluidic device capable of incremental, real-time monitoring without the necessity for expensive equipment or instrumentation.

In one embodiment, a method and system for qualitative detection of amplification product (either PCR or LAMP) are provided, e.g., a method and system for quantitative real-time monitoring of amplification reaction progress (PCR or LAMP), such as qPCR.

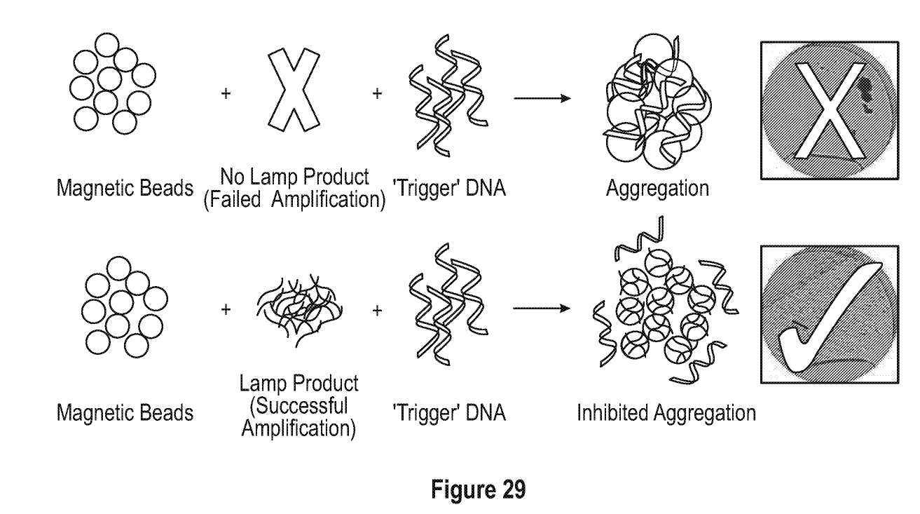

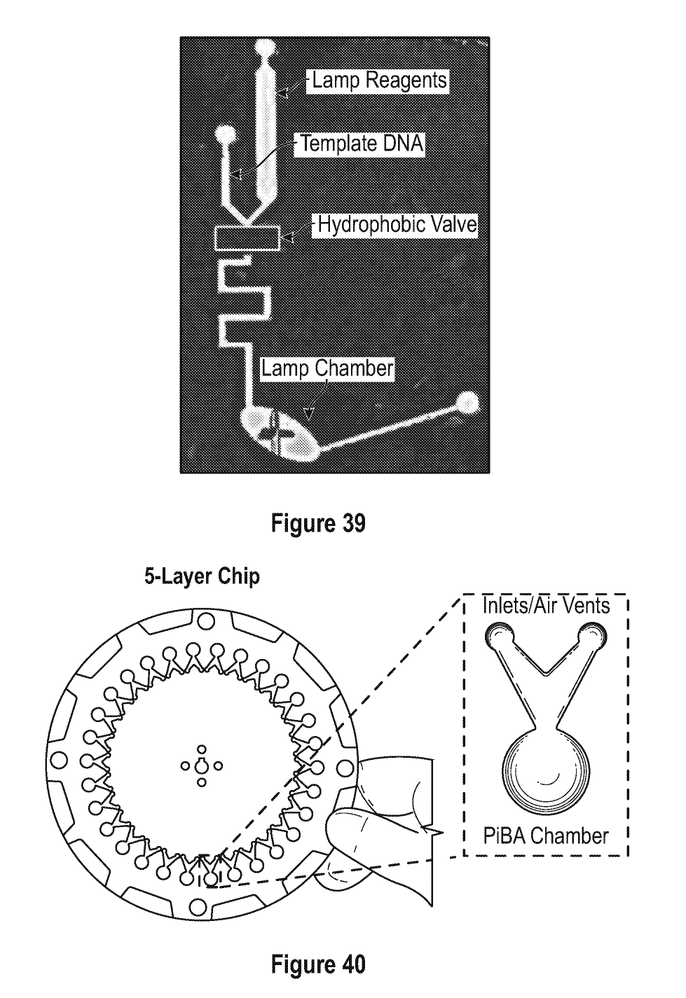

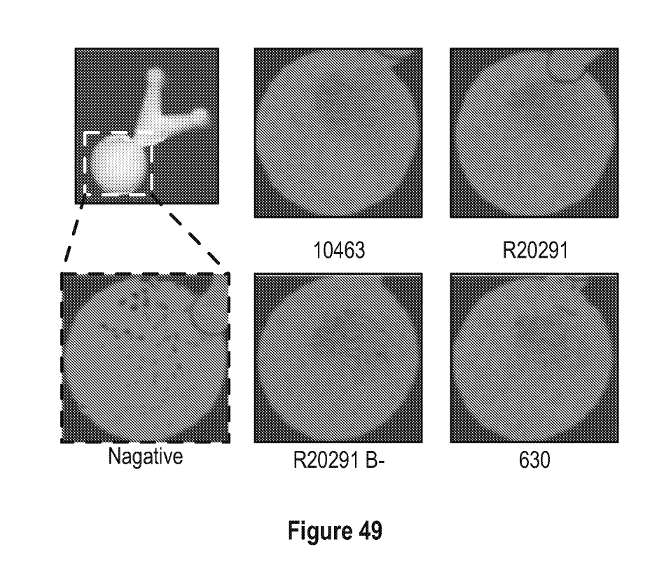

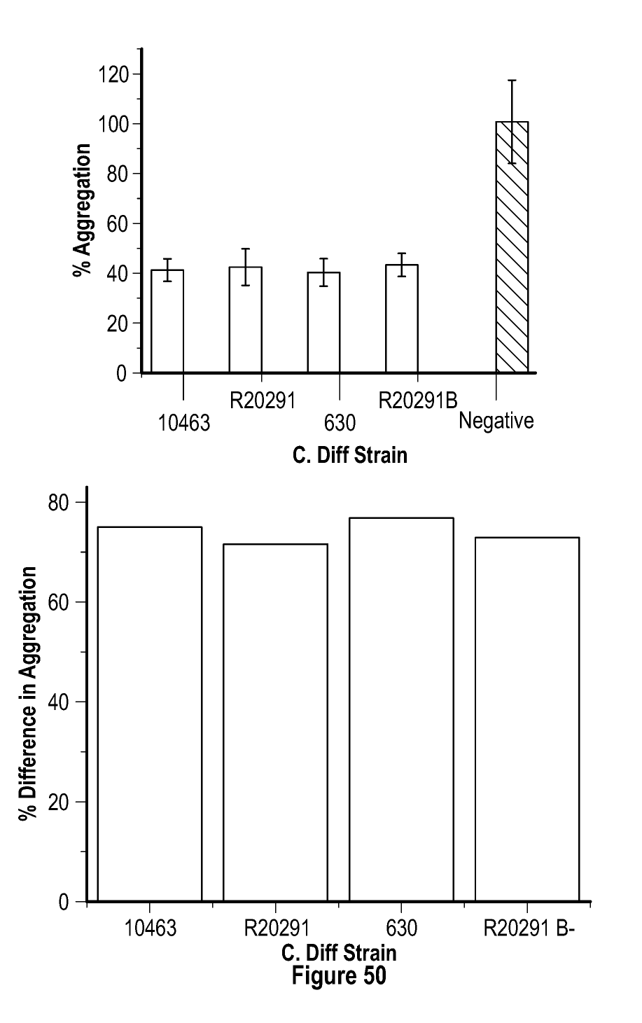

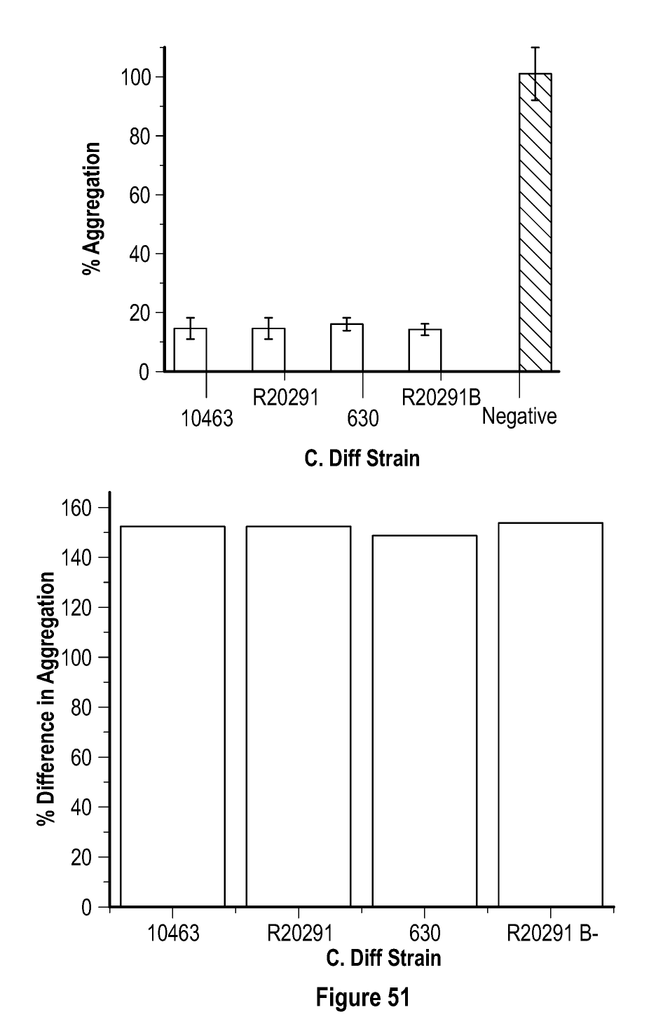

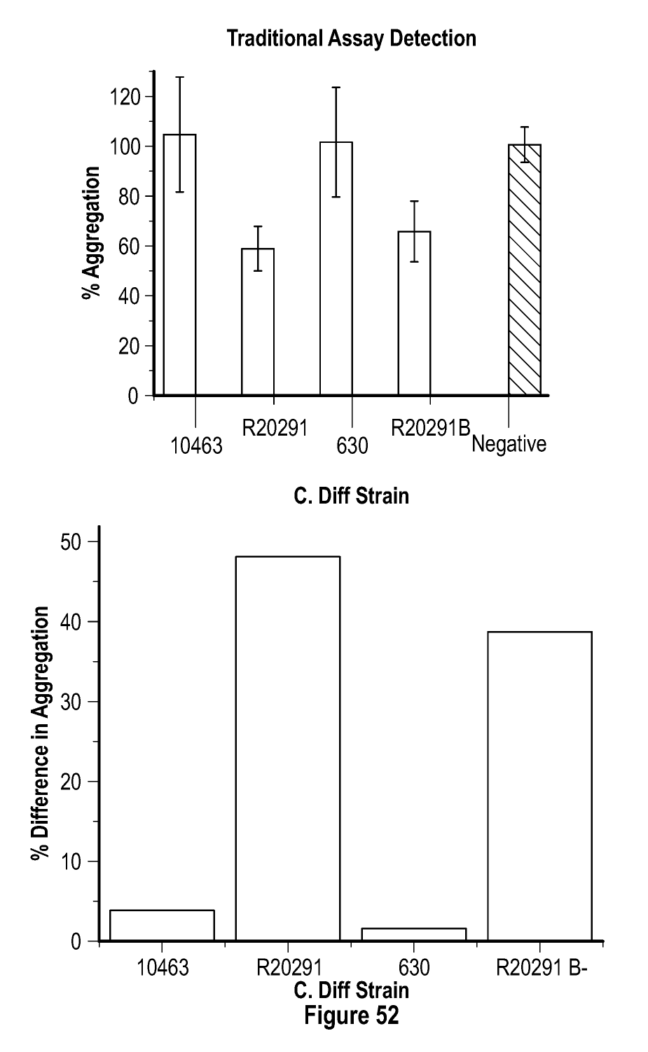

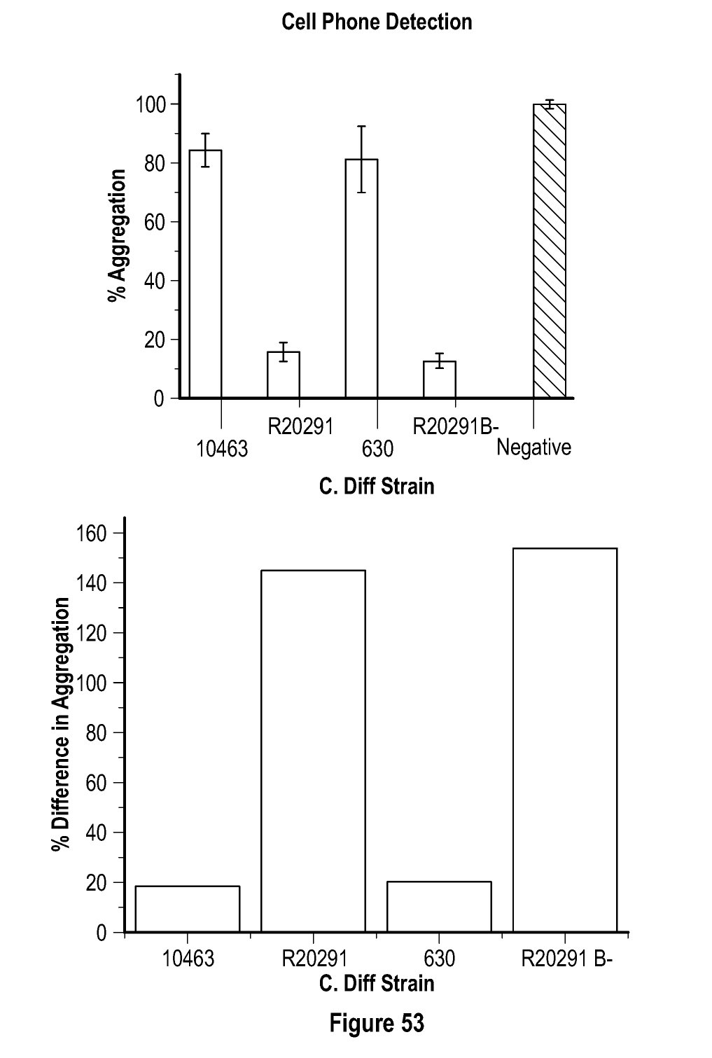

Further examples are shown in see FIGS. 29-53. Infectious diseases take a massive toll on populations. Many of these diseases are largely treatable and early detection very important. Currently, detection requires skilled personnel and expensive instrumentation/equipment. As described herein, lab-on-chip technology that is rapid and robust, sensitive and specific, user-friendly and portable is provided. Loop-mediated isothermal amplification produces billions of fragments of varied length over widesize range, and only one temperature is needed (65° C.). Moreover, multiple primers (6-8) can be used for increased specificity.

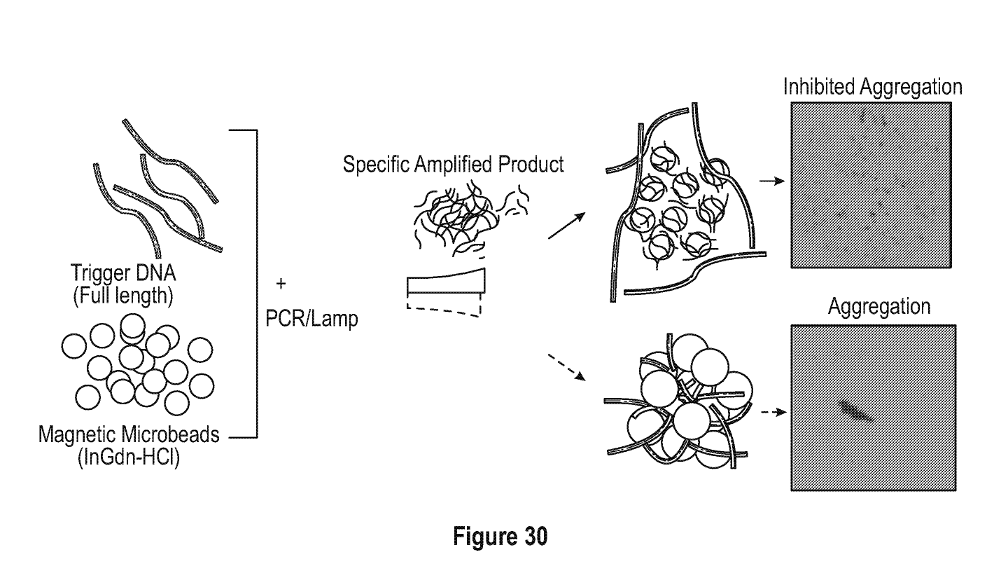

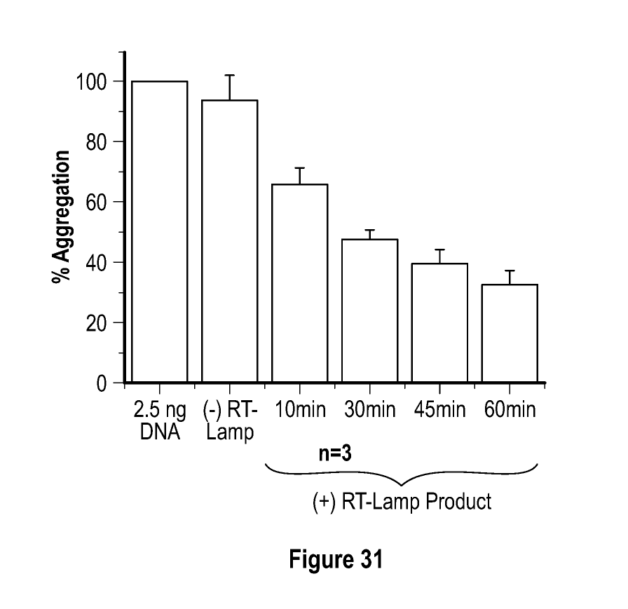

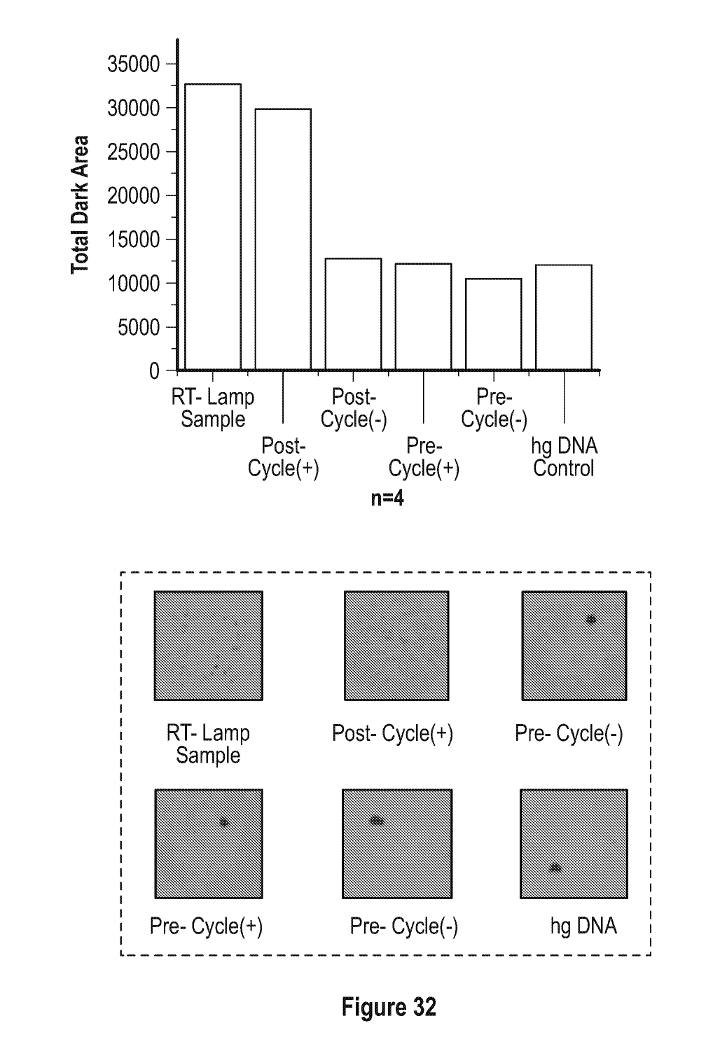

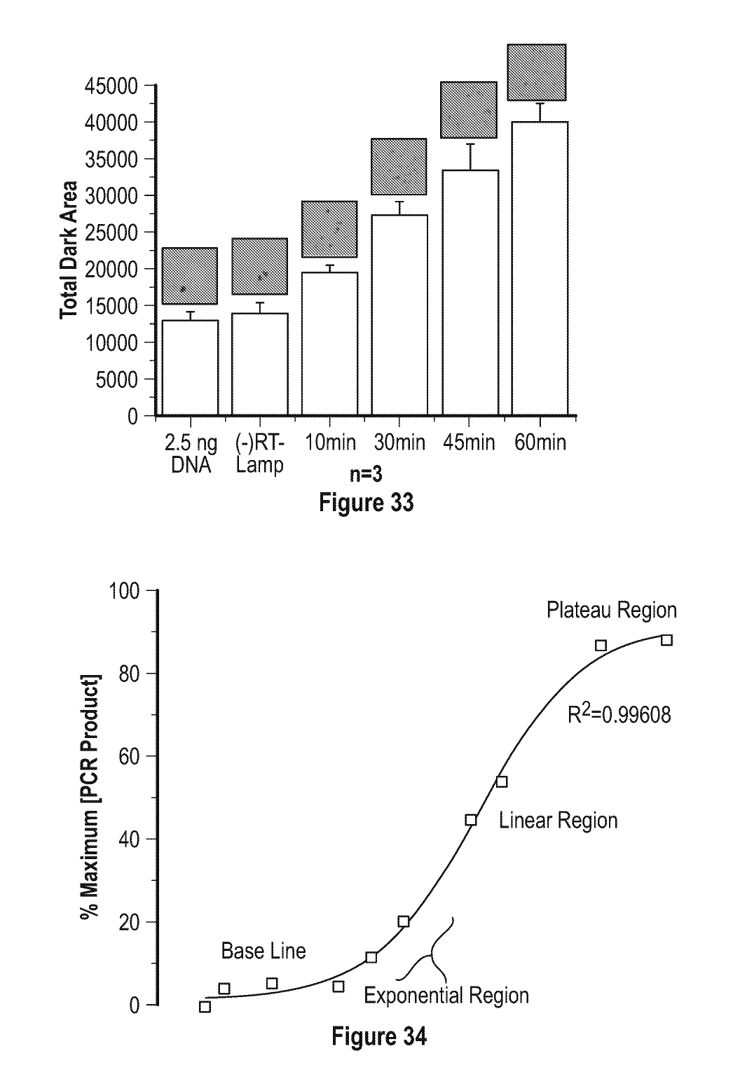

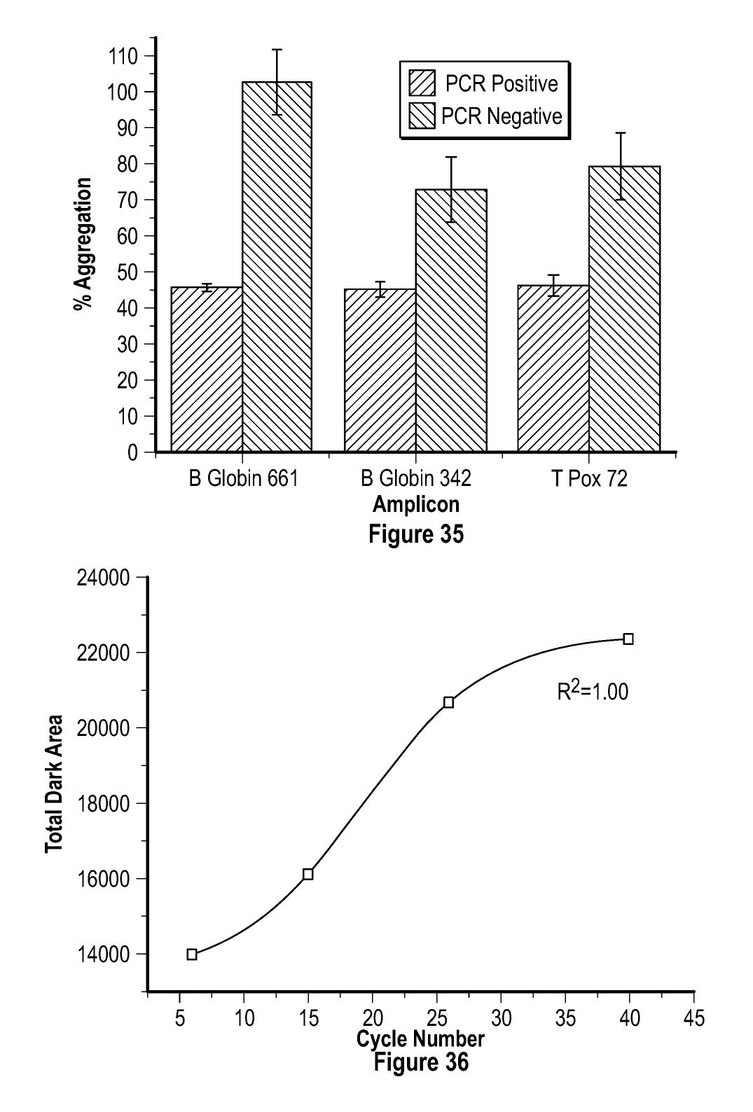

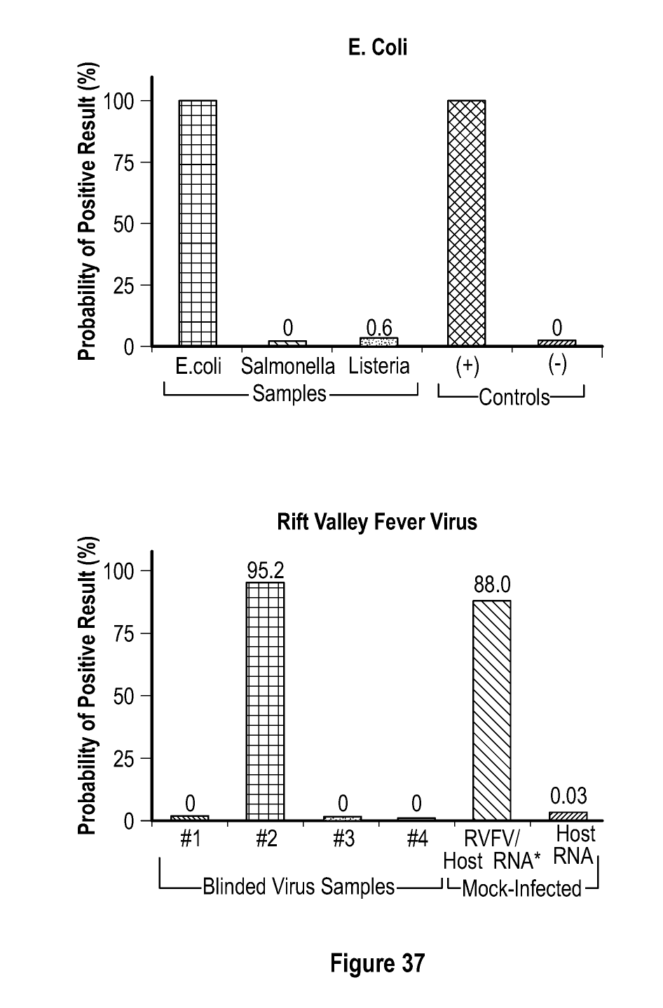

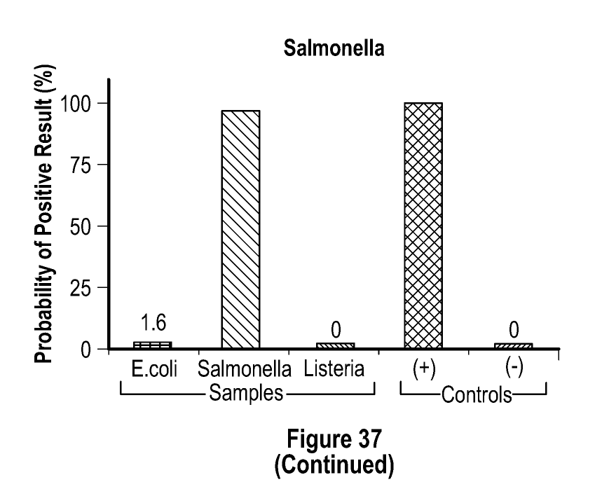

FIG. 29 is a schematic of PIA. Amplification product coats the magnetic beads and so full length DNA no longer induces aggregation. FIG. 30 is a schematic showing magnetic beads in Gdn-HCl add ‘trigger’ DNA and an aliquot of the resultant solution from amplification if aggregation occurs, no amplification product generated. FIG. 31 shows a bar graph of aggregation % and RT-LAMP product over time and FIG. 32 shows controls. FIG. 33 show a time course for aggregation inhibition. FIG. 34 shows results with RT-LAMP mixture sampling. The response resembles real-time fluorescence response for qPCR/LAMP. PAI can be used for qualitative detection of PCR products (FIGS. 35-36). FIG. 37 shows the use of PIA to detect various pathogens.

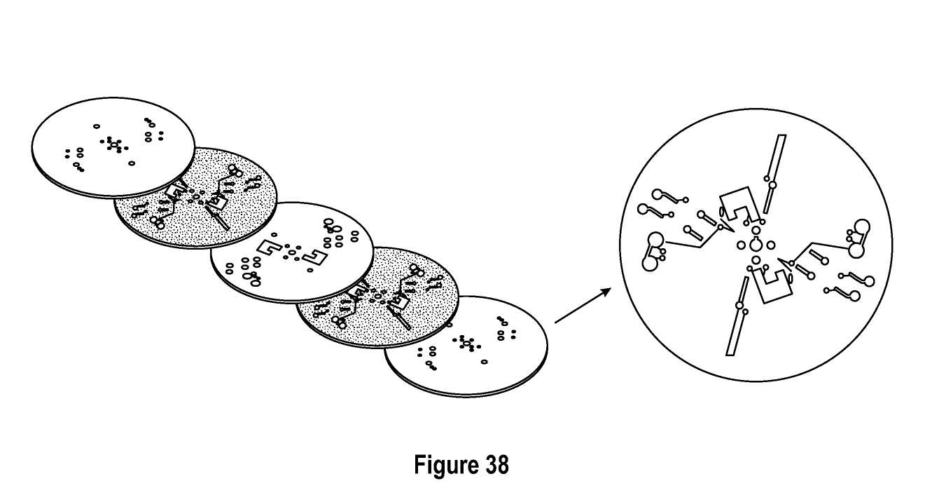

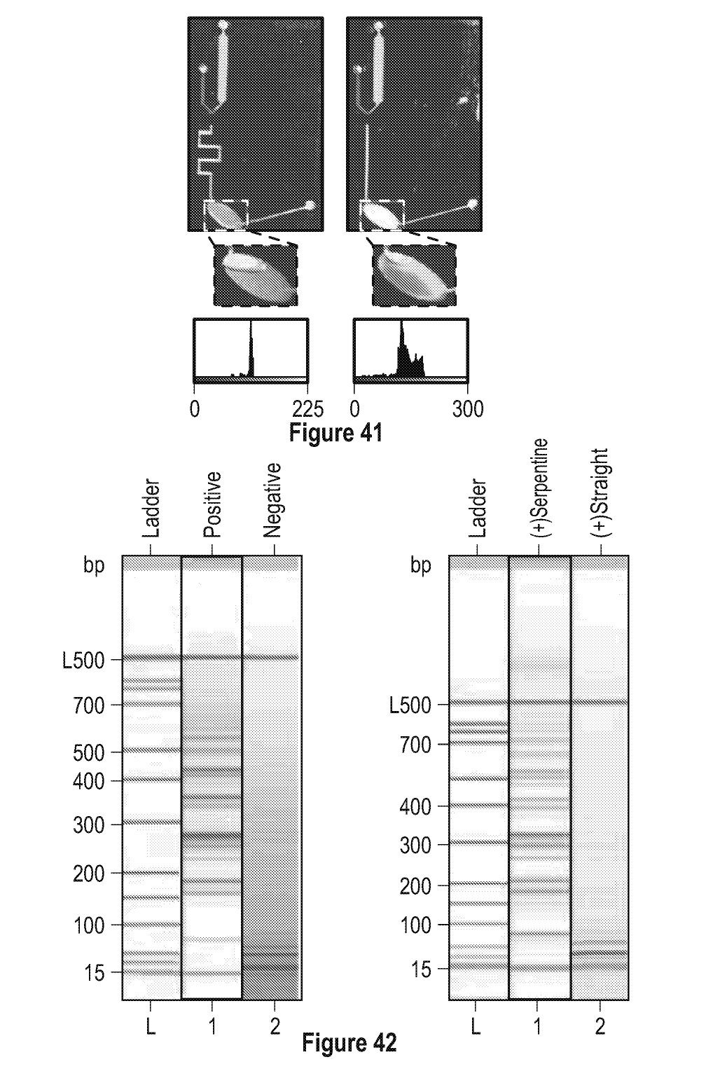

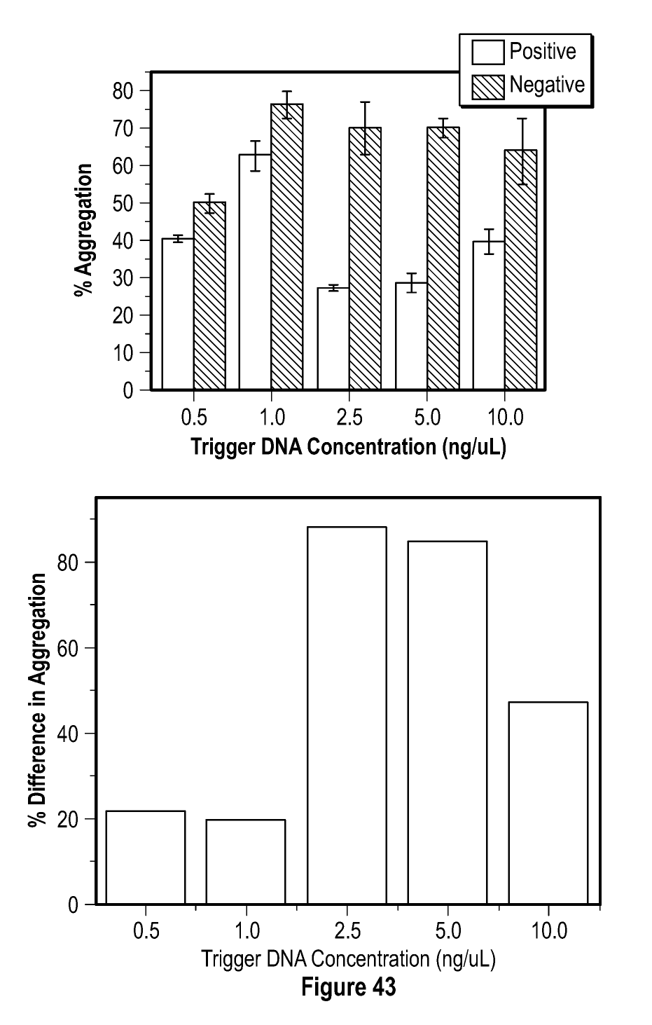

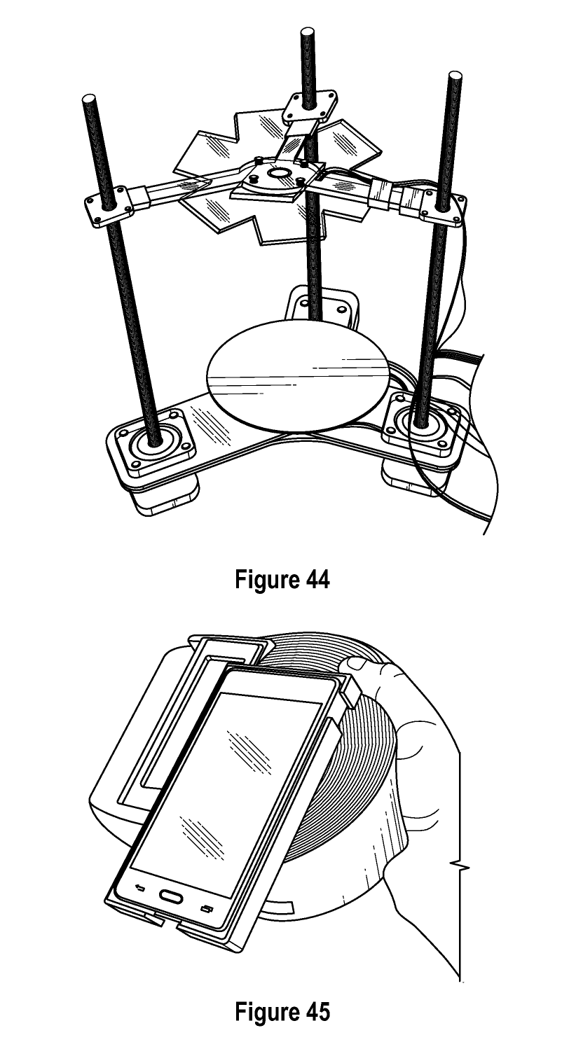

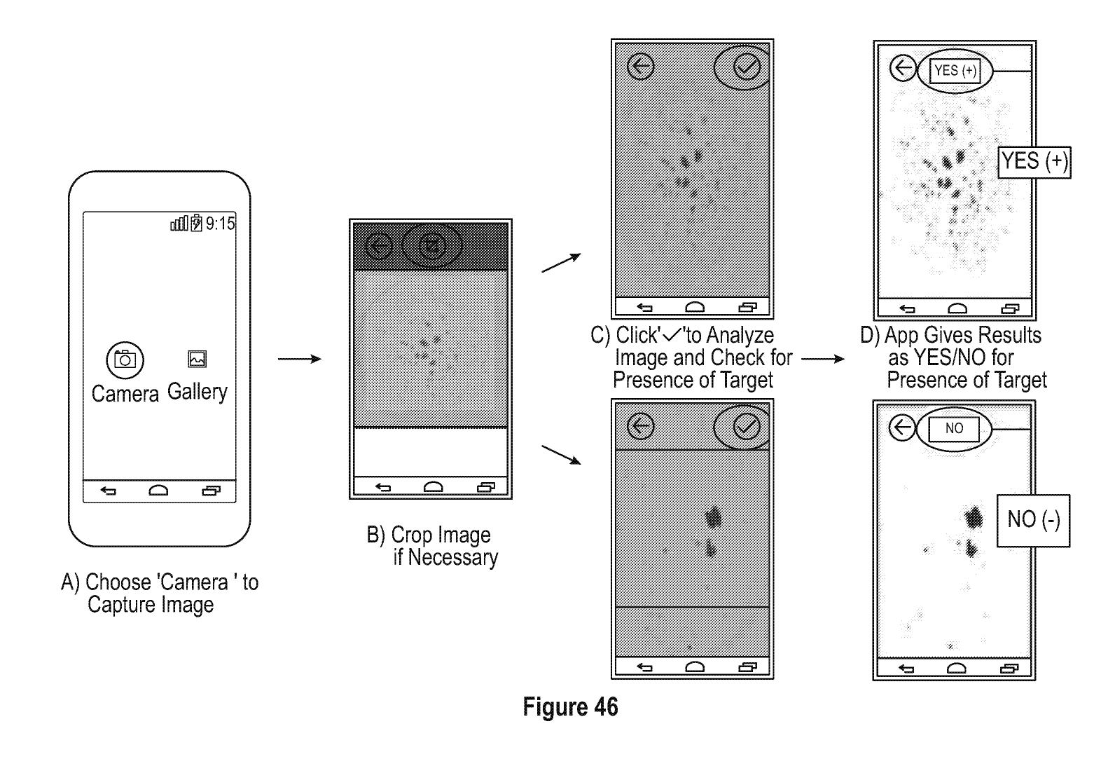

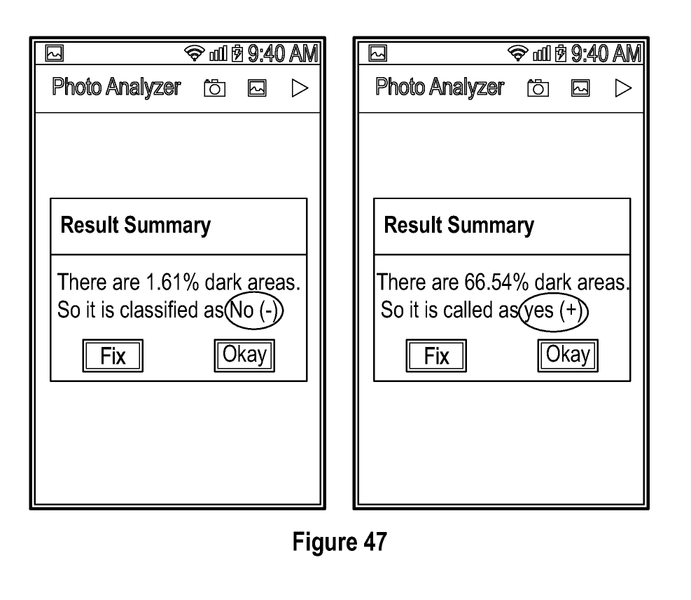

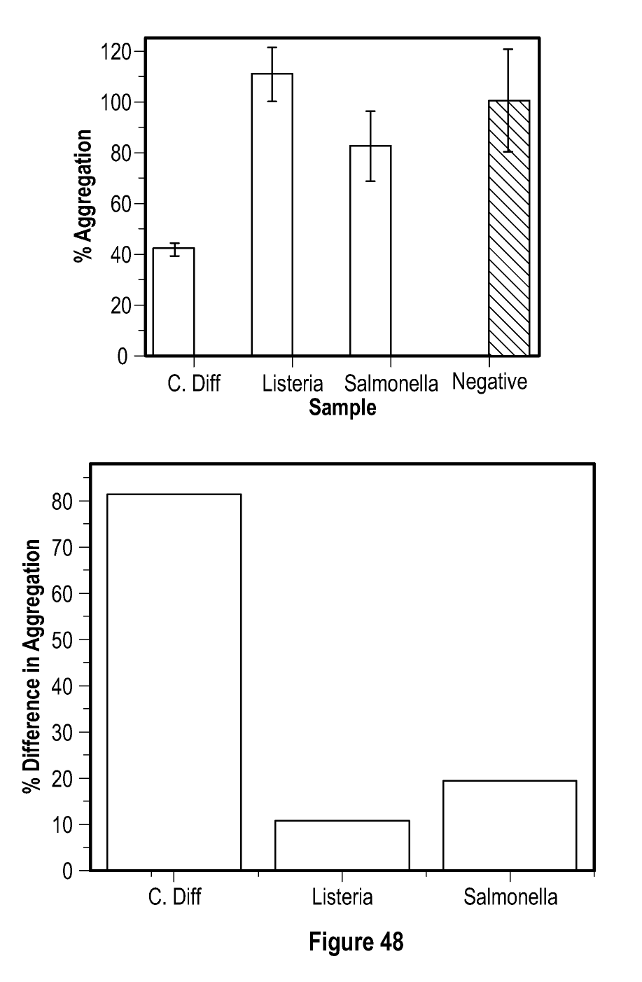

In one embodiment, a polyester toner microdevices may be employed (FIG. 38) in LAMP (FIG. 40). Serpentine channels may allow for improved mixing on a chip (FIGS. 41-43) in a device with a rotating magnetic field and spinning platform (FIG. 44). In one embodiment, a cell phone can be employed for image capture (FIGS. 45-46) and display of results (FIG. 47). For example C. difficile and specific strains thereof may be detected (FIGS. 48-53).

Prior to PIA, two methods allowed for the use of pinwheel formation: CIA, which provided for quantification of generic DNA in crude samples, and HIA, which provided form qualitative detection of specific targets post-PCR. In CIA under chaotropic conditions, only full length DNA produces aggregation, PCR products do not. In HIA, under physiological buffer conditions, only PCR products produce aggregation, full length DNA does not. Although PCR products are too short to entangle beads, they can bind to the beads. That binding inhibits aggregation of the beads by the longer DNA (PIA).

### Example 3

Messenger RNA (mRNA) markers can provide tissue-specific genetic information critical to areas ranging from early disease detection and cancer mutations to pathogen detection and sexual assault analysis. Primary detection methods for these markers include gel electrophoresis and fluorescence, which are reliable but require complex, expensive instrumentation and are very time-consuming methods. As a result, there is intense interest in simple methodologies that enable inexpensive and rapid mRNA detection.

Non-fluorescent detection schemes for genetic analysis are gaining momentum as a result of the potential for accelerating the sampling process using inexpensive detection systems that may be amenable to point-of-analysis regardless of the field. While mRNA marker detection touches a plethora of endpoint applications (e.g., early cancer detection, understanding cell physiology, and disease prevention), one area where there is dire need for an inexpensive detection system is in ‘presumptive body fluid identification’ (BFI).

This is a precursor to all forensic DNA processing and plays a pivotal role in providing contextual clues for crime scenes, particular with sexual assault cases, and contributes to the substantial backlog of rape kits that await analysis in the US (currently >80,000). While rapid colorimetric presumptive and confirmatory tests for individual body fluids have existed for some time, they are not reliable due to the lack of fluid specificity.

Other methods are surfacing using purportedly fluid-specific mRNA markers and exploiting fluorescence for the detection blood, semen, saliva, and vaginal fluid. However, these methods require lengthy sample preparation (6-24 hours), with some requiring statistical analysis to define a “probability” that a particular fluid is present. A method limited to only whole blood and requiring fluorescence detection, is of limited use for forensic analysis.

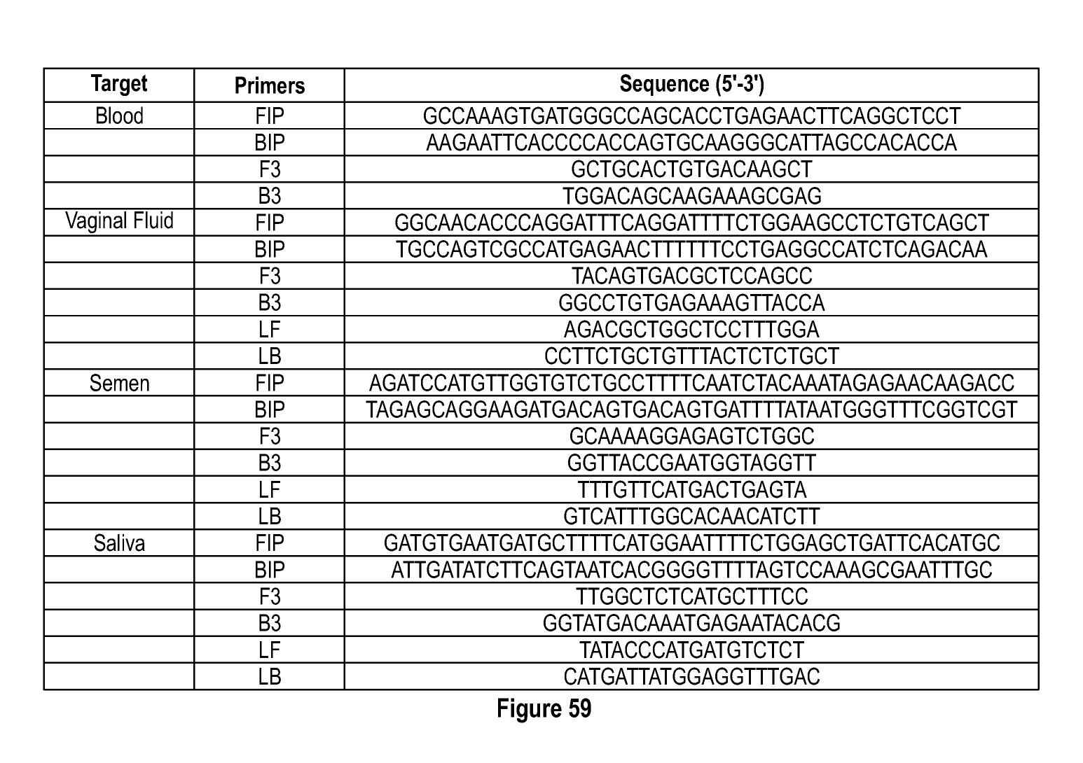

In one embodiment, three components are employed: cell phone (or other processor device) detection, image analysis, and automation into a prototype instrument. In all cases, for example, samples are processed with an optimized universal method for body fluids such as blood, vaginal fluid, semen, and saliva samples (sequences in FIG. 59). For LAMP amplification, samples are placed in a 96-well plate to allow for up to 23 samples to be processed simultaneously for all targeted body fluids. A metal chelating dye, hydroxyl naphthol blue (HNB), is placed in each LAMP assay prior to amplification and induces a color change from purple to a sky blue with successful amplification. This colorimetric change can be captured with a smart phone and processed via imagej analysis.

A custom photo box was designed to accommodate the focal distance for an iphone 6 but could easily be accommodated to fit other phones (or electronic devices or processor based systems/devices) of choice. Following the LAMP amplification, the 96-well plate was placed to fit inside of the top fitting of the photo box. A cell phone, resting against the bottom of the box, is moved forward until the full 96-well plate is fully visible by the smart phone lens. Images are captured of the plate with a thin white paper was placed over the 96-well plate to ensure a uniform background. The images from the phone were then transferred to image processing.

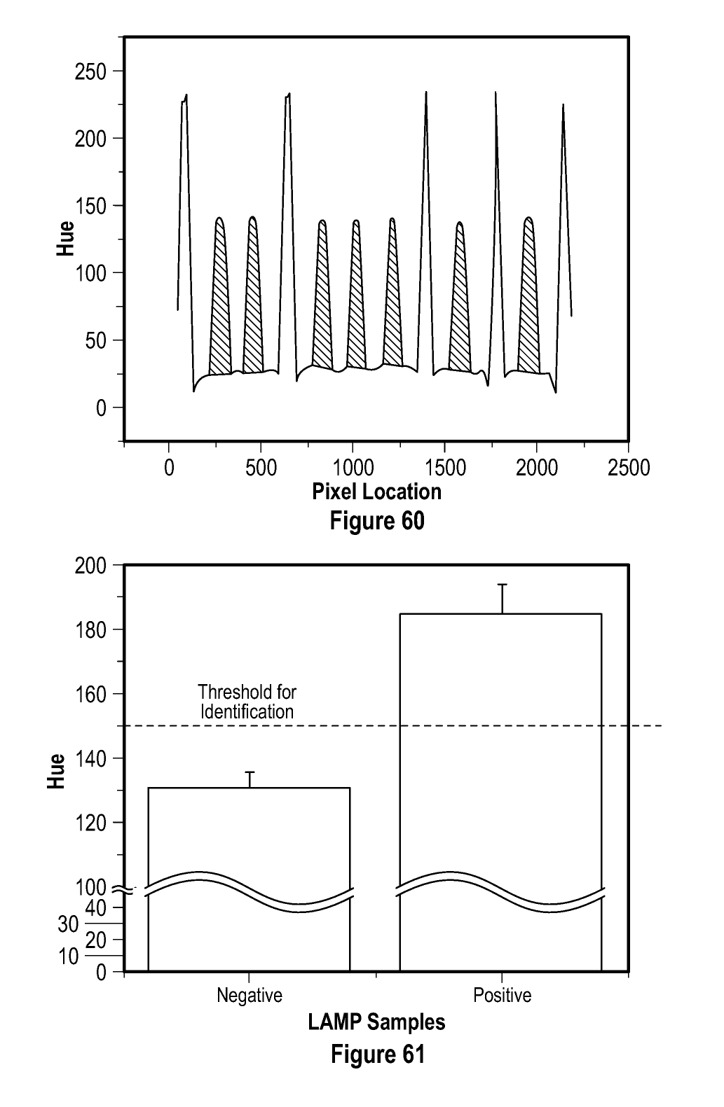

Imagej is open source imaging processing software used for image processing. Images, taken directly from the cell phone were initially imported into imagej and transformed to mimic what the plate would look like from the top. The images were then converted from the red, green, blue (RGB) cubed color space to a cylindrical hue, saturation, brightness (HSB) stack. This conversion provides advantages to processing color information because it can distinguish pure color (hue), the shade of the color (saturation), and the contribution of white light to the color (brightness). Of these color conversions, hue provided the greatest differentiation between violet and blue samples and was used. In this channel, the image is converted to an 8-bit grayscale and linearly scaled pixels between 0-255. A linear line is manually drawn across each horizontal row of the samples in the image and plotted. Since there is the inclusion of a negative control, it will ensure that amplification provides accurate results (no contamination) and can be used to set the baseline for all negative samples. These assigned hue values are then run through a moving average to minimize the edge effects from light refraction along the edges of the polypropylene tubes. Following these procedures, the present inventors determined that all amplified samples (blue) were assigned hue values around 130 whereas purple samples were assigned values closer between 180-200 as shown in FIGS. 60 and 61. A threshold was defined just over three times the standard deviation of the amplified samples (150 as shown FIG. 61) which could be used to automatically determine whether a sample amplified or not. In analyzing the hue values for a full 96-well plate, the present inventors determined this threshold of 150 to be accurate for assigning all samples. The surface plots were reproducible from multiple pictures of the same plate, of different plates, and even between days as long as the light source remained the same. Using this technique, the present inventors were able to correctly identify the fluids contained in 23 samples simultaneously in less than 3 hour).

This technology can easily be adapted to an automated platform and with only simple and inexpensive instrumentation. Since LAMP only requires a single temperature for amplification, a heater can be placed in the top of the box to heat the samples at the specified temperature without a thermocycler. Given a specified amplification time, a photo can be taken and analyzed directly through a custom phone app, or through a small microprocessor. From the threshold value, a user-friendly table could be displayed to reveal which body fluids are found in each sample of interest. Since the plate will be placed in the same location every time, a mounted camera can capture identical pictures of each plate. An algorithm (computer readable medium) can identify the location of every well and perform the image analysis to determine the appropriate hue. Examples 4 and 5 provide further details regarding LAMP.

### Example 4

Human identification based on STR analysis of DNA from biological samples is now routine practice, providing critical “source level” information. However, the DNA profile does not provide investigators with contextual or “activity level” information that can be critical to casework investigations. Forensic serology focuses on characterizing the biological fluid present on evidentiary material, and is now being viewed as a ‘first-pass screen’ prior to; and independent of, DNA analysis. Currently there is immense interest in a simple, high throughput method of rapidly detecting the standard fluids of interest—vaginal fluid, saliva, blood and semen. There is an additional need to be able to detect menstrual blood, as well as semen with or without sperm cells. Current fluid identification techniques fall woefully short on this front for a variety of reasons. They: (1) involve a significant number of manual steps, (2) have long processing times, (3) require expensive, complex analysis methods, (4) are challenging for high throughput scaling, (5) may require statistical analysis, and (6) have low accuracy (esp. saliva and vaginal fluid). Moreover, none of the colorimetric tests currently available can simultaneously detect all six of these fluids.

The BodyFluID System is capable of performing accurate body fluid identification (bf-ID) in a 96-well format with simple bioanalytical methods and a low-cost instrument. With any given sample, the assay currently defines the presence blood, semen, vaginal fluid or saliva, alone or in combination. Menstrual blood and cell-free semen may be included, with the Sample-to-body fluid II) time of 90 min for one sample, or <3 hrs for 23 samples (for 4 fluids; 16 samples for 6). Additionally, this simple process fits within the existing workflow for STR analysis and consumes so little sample that the same extraction used for bf-ID can be used for STR amplification. This will directly provide investigators with information on DNA-containing crime scene samples of interest and contextual information on the activities that allowed for the transfer of biological fluid.

The BodyFluID System is capable of processing a 96-well plate (23 samples) with manual colorimetric analysis as well as with an automated colorimetric analysis capability and objective read-out of the results. The accuracy of the system has been demonstrated and the assay can complete the sample-to-fluid ID process for a 96-well plate in <3 hrs, and the device consumes about <1 ft² of benchtop space.

The BodyFluID System dramatically increases speed, throughput, and accuracy of bf-ID with a simple, low-cost, fluor-free approach. In one embodiment, heating and image capture are integrated, eliminating the 96-well plate post-reaction transfer. The image capture conditions and software translate manual image analysis to an automated process, allowing the user to interface with a simple GUI.

In one embodiment, the instrument incorporates an isothermal heat block underneath the image capture area. In one embodiment, image capture conditions ensure maximum colorimetric discrimination. In one embodiment, image analysis software automatically interprets color change and reports body fluid ID. In one embodiment, a GUI interface for sample input information and assay result interpretation is provided. In one embodiment, the assay allows for high throughput, low-cost, confirmatory screening for six body fluids (whole blood, menstrual blood, saliva, vaginal fluid, cell-free semen and sperm cells) that provides an order of magnitude reduction in analysis time relative to methods currently available. The GUI interface automatically processes the results to provide the user with body fluid information.

Loop-mediated isothermal amplification (LAMP) for both DNA and RNA, and automated colorimetric analysis were thus applied to an assay that involves simple lysis/DNA extraction, followed by a <30 min isothermal amplification with colorimetric smart phone readout. The system is capable of saliva, blood, semen, and vaginal fluid, alone or in combination.

### Example 5

Genetic DNA profiling via short tandem repeat (STR) analysis is heavily relied on for unique identification of an individual in a criminal case. Body fluid identification (BFI) has gained recent attention due to important contextual information that is not provided through STR analysis. The identification of saliva versus semen on a victim's clothing, for instance, will significantly change the focus of an investigation. To be effective with the current DNA workflow, BFI methods should be non-destructive, sensitive and accurate, and with high specificity.

Traditionally, forensic labs rely on presumptive tests followed by confirmatory testing for the identification of blood, saliva, and semen. These presumptive tests interact with the body fluids to produce a colorimetric change, visible to the eye. Although these tests are sensitive and provide rapid results, they are not human specific and are susceptible to false positives. With increased specificity, confirmatory tests can provide a colorimetric answer via antibody-antigen interactions with increased confidence. However, even the most popular immunological test suffer from false positives so there still remains a need for improved BFI methods.

Messenger RNA (mRNA) profiling is a promising method for identification of biological material due to mRNA stability and DNA workflow compatibility via co-purification of RNA with DNA. Forensically-relevant body fluids such as vaginal fluid and menstrual blood could not be previously be identified via catalytic or enzymatic methods but are now ascertained via mRNA markers such as MUC4 and MMP7, respectively. Although multiplexed mRNA profiling assays have been reported by methods including quantitative real-time polymerase chain reaction (q RT-PCR) assays or high-resolution melt (HRM) analysis, these methods rely on expensive instrumentation and may provide false negatives or false positive identification.

A more recent Nanostring® barcoding system uses 18-23 different mRNA targets and probe hybridization to identify fluids such as blood, semen, saliva, and vaginal fluid, and sweat. Accurate identification of blood and semen were possible using mRNA markers, however, due to the use of multiple non-specific mRNA markers, a statistical algorithm had to be used for determining the ‘probability’ of a stain containing either saliva, vaginal fluid, or sweat. In addition, this method required large sample volumes (50 μL stains) in combination with a lengthy 12-24 hour hybridization time frame per sample before body fluids could be identified.

An alternative and simple BFI method was proposed for rapid testing of blood used real-time detection reverse-transcription loop mediated isothermal amplification (RT-LAMP). The high specificity, rapid isothermal amplification, and single-tube approach of LAMP detection via turbidity measurements eliminated the false identification encountered with RT-qPCR and simplified the sample processing. This method can accommodate even trace samples down to 10⁻⁵ ng of RNA but only applies to blood samples and has yet to be demonstrated with any other body fluid.

Here, a body fluid panel is provided for the identification of blood, saliva, vaginal fluid, semen, and azospermatic semen samples using RT-LAMP coupled with a simple optical detection method. A single fluorescent-free operating procedure was optimized for the simultaneous identification of all of the aforementioned body fluids in a simple manner. The addition of a metal-indicator dye, hydroxyl nathphol blue (HNB), provides a visual colorimetric readout of LAMP reactions that can be captured by a smart phone. Placement on a 96-well format increases the sample efficiency to provide for a sample-to-answer time of about 1.5 hours for up to 23 samples simultaneously.

### Materials and Methods

### Sample Collection.

Whole blood samples, provided by the University of Virginia Medical School, were collected via a standard venipuncture technique as a part of routine care and treated with 5.4 mg of K₂EDTA for anti-coagulation. These samples were collected biweekly and stored at 4-8° C. until used. All other de-identified buccal swabs, vaginal swabs, and semen samples were collected using procedures approved by the University Institutional Review Board (IRB). Freshly ejaculated semen samples were aliquoted into 50 μL volumes and stored at 4° C. until needed. Fresh de-identified vaginal and buccal swabs were dried and stored in a dark drawer at room temperature. To prepare dry stained samples, the body fluids of interest were manually spotted on 2″×2″ squares of blue denim, dark denim, a dark blue sheet, or cotton material and kept in a dark drawer overnight at room temperature. Stained sample volumes included 10 μL blood or semen, or wiping a fresh saliva or vaginal swab on the material for 30 seconds.

Azospermatic sperm samples were processed from collected ejaculated semen samples. Approximately 50 μL aliquots were placed in a 0.2 mL PCR tube and centrifuged at maximum speed (13,400 revolutions per minute) for 10 minutes. The supernatant was removed and placed in a separate 0.2 mL PCR tube. This process was repeated for another 5 minutes and the supernatant again placed in a clean 0.2 mL tube. Aliquotes from each sample was stained with SYTO 11 and examined under a fluorescent microscope (Zeiss Axio Scope.A1; Carl Zeiss Microscopy Ltd.; Jena, Germany) to ensure that there were no sperm cells prior to further sample processing. All samples were stored at 4° C. until needed.

2.2 RNA Isolation.

A Qiagen RNeasy Mini kit (Valencia, Calif.) was used for all purifications of samples using manufacturer instructions. To lyse blood, vaginal swab, buccal swab, or semen samples, 350 μL of RLT buffer was combined with 90 μL RNA-free water, 10 μL Proteinase K (Qiagen, Valencia, Calif.), and 4.5 μL of β-mercaptoethanol (Sigma Aldrich, St. Louis, Mo.). The samples were then incubated at 56° C. for TO minutes. All swab samples were placed in a 0.5 mL tube that had been punctured with a 21 gauge needle in the bottom of the tube. These tubes were placed in a 1.5 mL microcentrifuge tube and centrifuged at a short spin cycle for 1-2 seconds at maximum speed. The fractions spun through to the 1.5 mL tube were combined with the original lysed sample. The RNeasy kit manufacturer instructions were used for the remaining RNA purification steps. Once finished, all samples were stored at −20° C. until needed.

### Messenger RNA Marker Selection and LAMP Optimization.

B-globin (HBB; accession no. NM000518.4) was selected for blood identification, human beta-defensins (HBD-1; accession no. 25 NM005218.3) for vaginal fluid, human semenogelin-1-precursor (SEMG1; accession no. NM003007.4) as a semen marker, and histatin-3 precursor (HTN3; accession no. NM000200.2) for saliva detection. All LAMP primers were designed using Primer Explorer V4 and purchased from Eurofins MWG Operon (Huntsville, Ala.). A Loopamp DNA amplification kit (Eiken Chemical Co., Ttd, Tokyo, Japan) was used in combination with a reverse transcriptase (RT) kit (High Capacity RNA-to cDNA™ kit; Thermo Fisher Scientific, Waltham, Mass.) for individual body fluid LAMP optimizations and specificity testing according to the manufacturer instructions. Reaction volumes were reduced to 5 μL and consisted of 1× reaction mix (40 mM Tis-HCl (pH 8.8), 20 mM (NH4)2SO4, 16 mM MgSO4, 20 mM KCl, 0.2% Tween, 1.6 M Betaine), 20 pmol LF and LB primers, 5 pmol for F3 and B3 primers, and 40 pmol for FIP and BIP, and 8 U Bst polymerase. Approximately 0.5 μL sample volumes were added to reaction volumes. A Biorad MyCycler Thermal Cycler (Bio-Rad Laboratories Inc., Hercules, Calif.) was used for all amplifications. Initial LAMP reactions were examined visually for increased turbidity and analyzed on Agilent 2100 instrumentation using DNA 1000 series II kits (Agilent Technologies, Santa Clara, Calif.) for confirmation of amplification. Once individual LAMP reactions were optimized, all LAMP amplifications transitioned to a Loopamp RNA Amplification kit (Eiken Chemical Co., Ltd, Tokyo, Japan). Reaction volumes were increased to 10 μL for optimized colorimetric detection of LAMP with all previously mentioned concentrations remaining the same. Approximately 1 μL of sample was placed in each LAMP reaction.

Colorimetric LAMP Analysis.

Hydroxynaphthyl blue (HNB) dye (Sigma Aldrich, St. Louis, Mo.) was added to each LAMP reaction to a final concentration of 120 μM in an amplification volume of 10 Samples were amplified in 96-well plates (cat #2239441; Bio-Rad Laboratories Inc., Hercules, Calif.) at 63° C. for up to a 60 minute amplification, with a 2 minute incubation at 95° C. to denature the BST polymerase. Once the amplification was finished, the 96-well plate was placed inside of an in-house built photo box made of poly methyl methacrylate (PMMA). Images of all sample wells were taken with an Iphone 6 (Apple Inc, Cupertino, Calif.) cell phone. The images were analyzed in Image) software using a hue, saturation, brightness (HSB) filter (HSB stack) surface plot to capture a hue profile for each well.

### Results

### LAMP

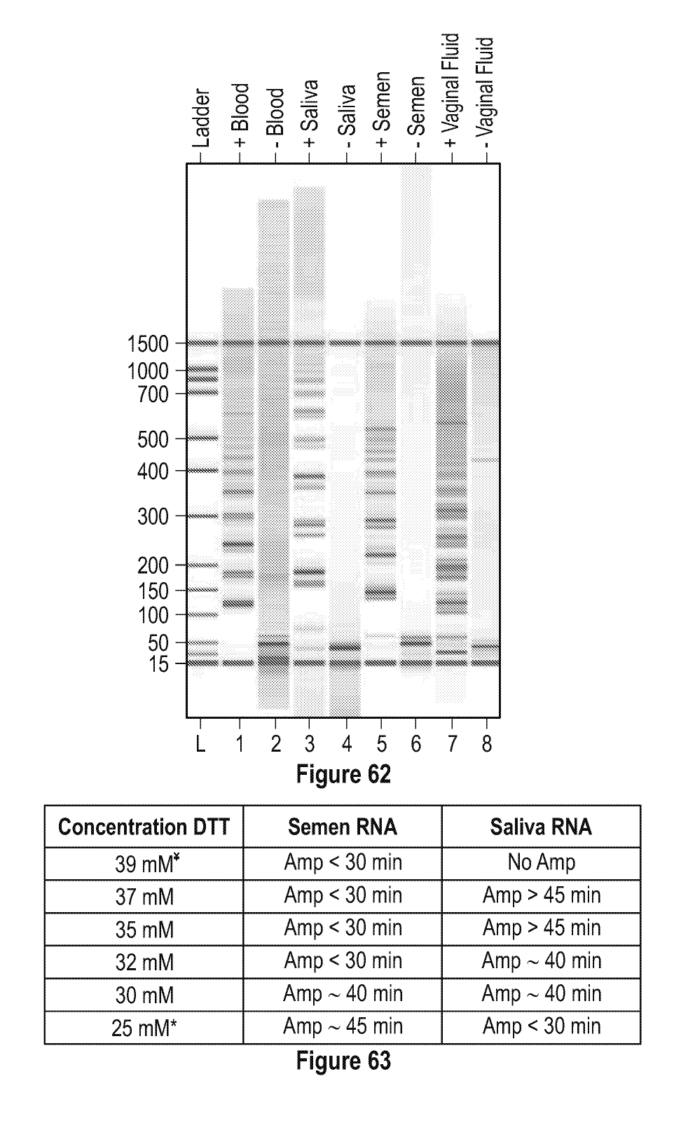

A unique mRNA marker for each body fluid was chosen based on fluid specificity and include HBB (blood), HBD-1 (vaginal fluid), SEMG1 (semen), and HTN3 (saliva) (FIG. 59). Two of the five designed primer sets were chosen for each fluid in initial BFI testing. Extracted cDNA samples from each body fluid was used to test designed primers for successful amplification at temperatures between 60-65° C. Each of these primer sets did amplify the targeted body fluid at one or more temperatures. FIG. 62 shows an example electropherogram of the successful amplification of all body fluids at 63° C. using one of the two selected primer sets.

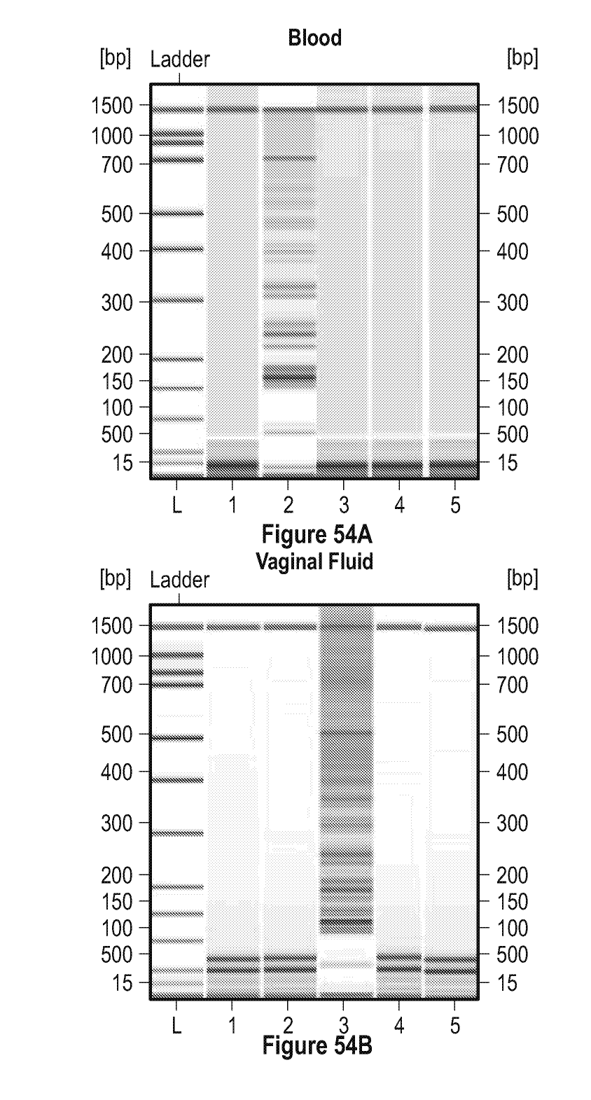

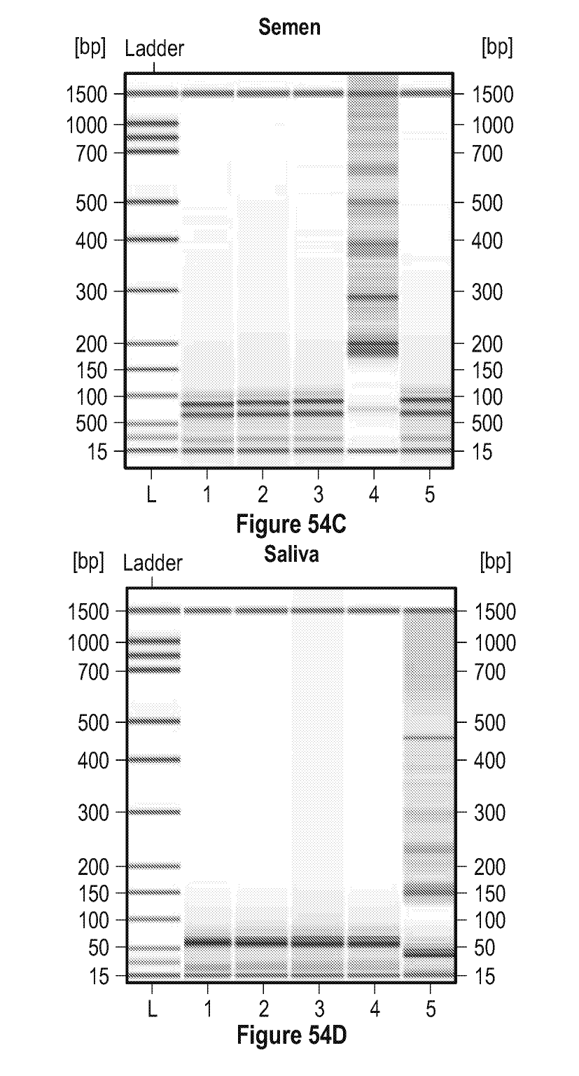

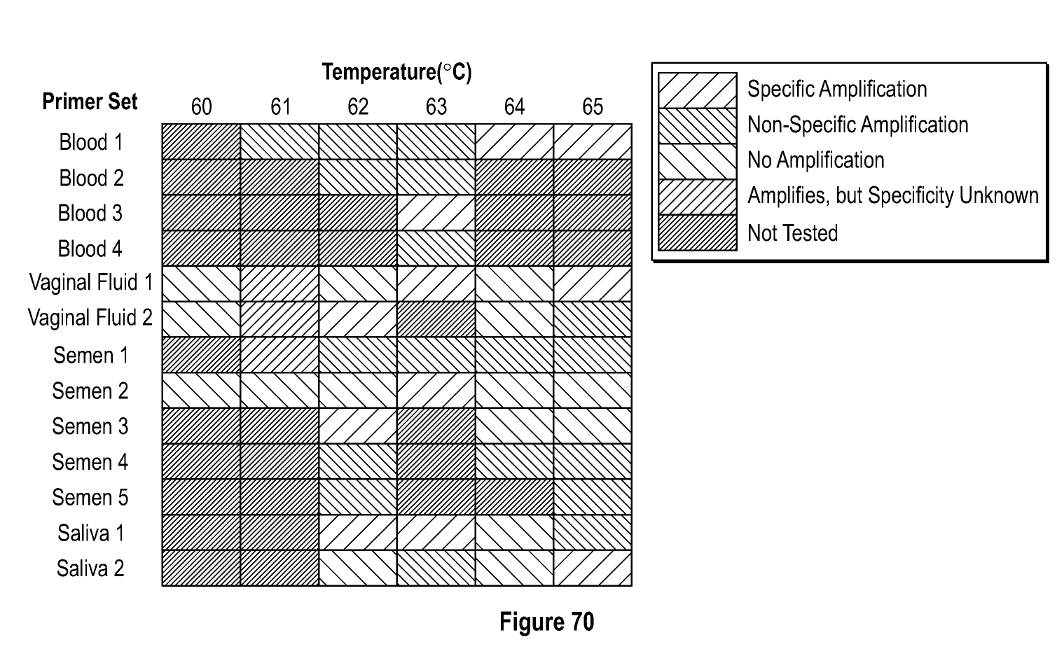

Although the designated primers allowed successful amplification of targeted body fluids, the specificity of each assay needed to be analyzed. To do this, cDNA from each body fluid was tested against each primer set, with the inclusion of a negative control (containing only LAMP reagents with Tris-EDTA (TE) buffer) and a non-specific positive control (1 ng pre-purified human genomic DNA). Despite the high specificity that LAMP amplifications offer, three of the four primer sets amplified non-specifically at 63° C. An amplification map was created to find a set of primers that could selectively amplify each targeted body fluid over a 60-65° C. range (see FIG. 70). Using this map, w three of the four body fluids were specific at both 63° C. and 65°, therefore, the number of primer sets were expanded for semen and blood. With this expansion of primer assays, full specificity of all targeted body fluids at 63° C. (n=20) was achieved as shown in FIG. 54. To shorten the sample-to-answer time for BFI identification, a RT-LAMP kit was used for direct amplification from RNA-extracted samples. This has been used from RNA samples for bacteria and human-derived materials with rapid results Changing from a LAMP kit to a RT-LAMP kit eliminated a 60 minute conversion of RNA to cDNA and did not negatively affect the efficiency of amplification (data not shown). Due to improved sample processing, the RT-LAMP kits were used for all future amplifications.

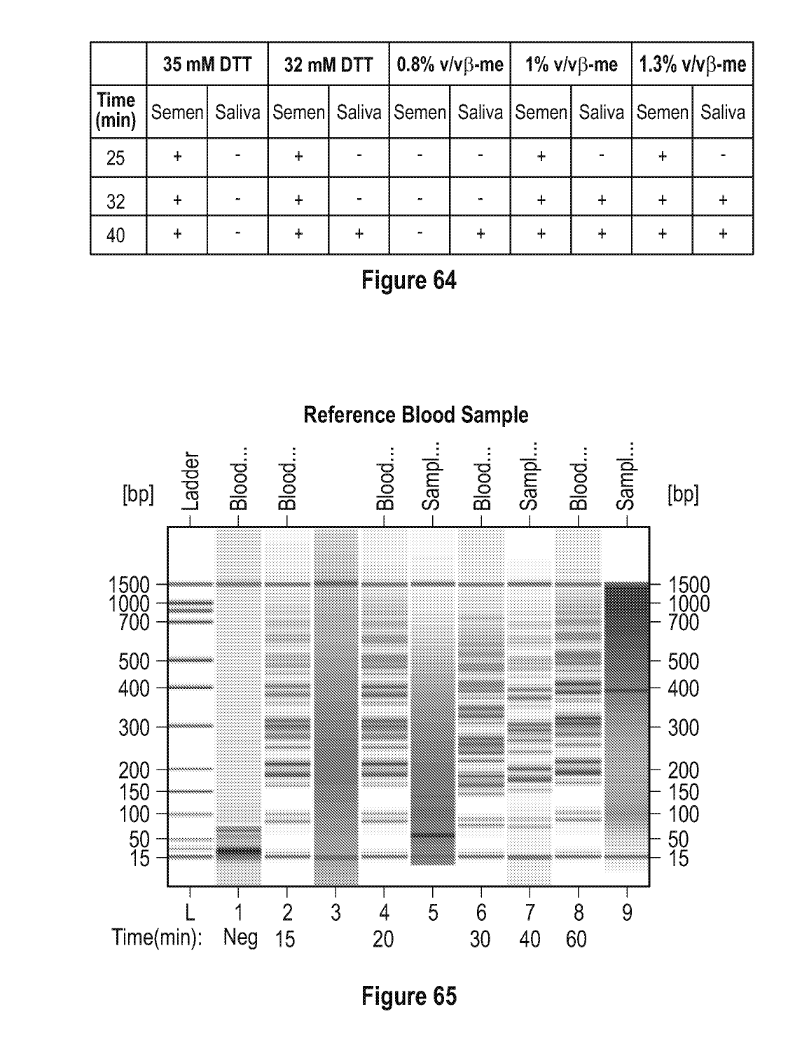

Universal Lysing Procedure. To detect all body fluids simultaneously, it was critical to develop a single universal sample preparation procedure for the lysis and extraction of all samples. Thus far, sample lysis were done according to different recipes. All recipes required different volumes of Qiagen RLT lysis buffer with 0, 20 mM, or 40 mM dithiothreitol (DTT) and the occasional use of Proteinase K (for vaginal swab and saliva samples). After e experimentation, 350 μL of Qiagen RLT buffer, 0.2 mg proteinase K, and 35 mM DTT was found to be useful for the lysis of semen samples (data not shown). When blood, semen, and vaginal swab samples were lysed with this recipe, all fluids reproducibly amplified within 30 minutes (n=10). However, the saliva LAMP assay weakly amplified at 60 minutes or failed to amplify at all (n=10). In a control study, it was found that the amplification of saliva was hindered with increasing DTT concentrations (FIG. 63). As DTT is necessary for the lysis of sperm cells, it was important to find a minimum concentration to lyse the sperm cells without inhibiting the amplification of saliva samples. FIG. 63 shows that 32 mM was the optimal concentration of DTT for semen and saliva samples, but only allowed saliva samples to amplify around 40 minutes. Another reducing agent, 2-mercaptoethanol (β-me), was explored as an alternative to DTT because of its known compatibility with Qiagen chemistry. FIG. 64 shows a comparison of a series of sample lysis protocols containing optimal DTT concentrations (32 and 35 mM) and concentrations of 0.8%, 1% (manufacturer recommended), and 1.3% β-me (n=3). Overall, samples containing ≥1% β-me provided more efficient amplification of saliva and semen samples than either concentration of DTT. Blood and vaginal fluid samples were tested in triplicates at 1% v/v β-me and also reproducibly amplified in less than 30 minutes (data not shown). As β-me provided more efficient amplification for all body fluids, it was integrated into the lysis for all future sample processing.

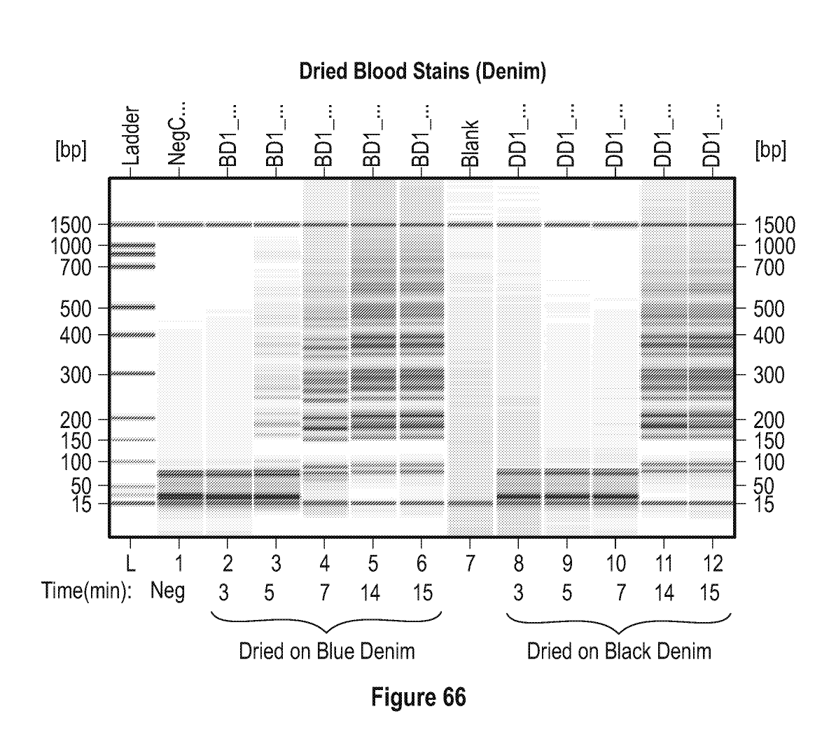

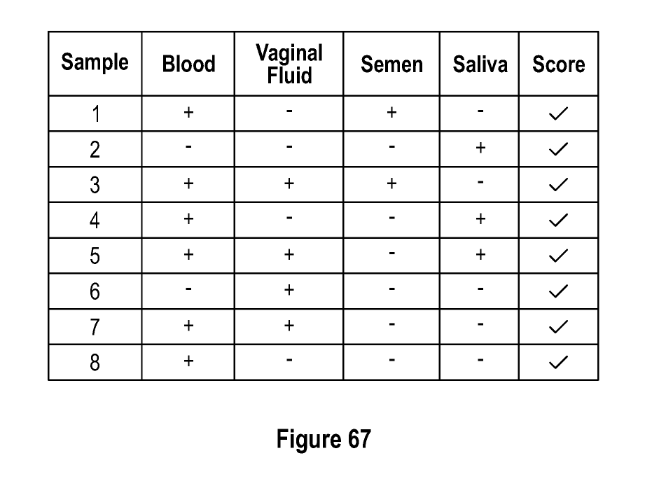

Dried Sample Stains. With a universal method established for all body fluids, the LAMP amplification method was challenged with difficult samples that cannot be easily tested by conventional chemical and enzymatic methods. These samples include dry fluid stains on denim and dark synthetic dye materials that can interfere with the visual interpretation of conventional colorimetric tests. Even though LAMP amplifications have been shown to be incredibly robust towards PCR inhibitors, the extraction of RNA prior to LAMP amplification will likely eliminate the presence of any inhibitors. For proof-of-feasibility, 10 μL of whole blood was spotted on 2″×2″ cuttings of the blue and black denim materials and dried overnight (n=3 each for 2 donors). From just a 1 mm×1 mm square cutting of the dried stain, blood could be identified in all samples within 15 minutes. A comparative example between whole blood (reference control) to dried blood samples is shown in FIGS. 65-66. Unknown dried mixtures containing combinations of blood, vaginal fluid, semen, and saliva were prepared to challenge the LAMP assays as a BFI panel. A series of 8 samples were prepared and blindly tested and interpreted by a second user. The sample interpretation of these blind samples, shown in FIG. 67, correctly matched all of the sample preparation and could be identified within a 30 minute amplification.

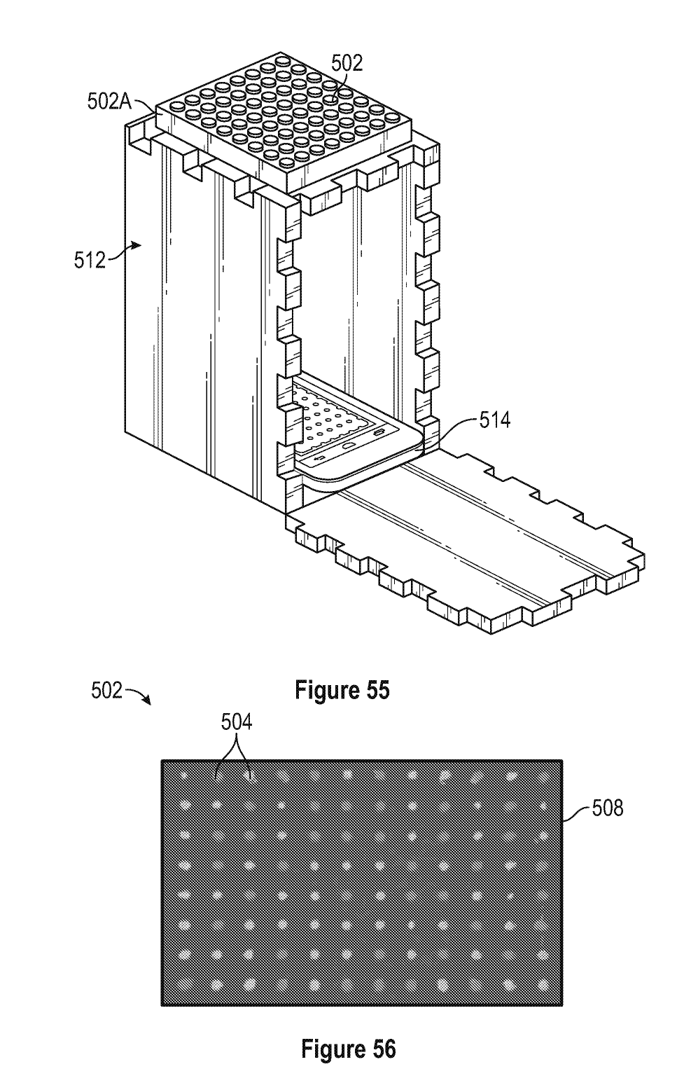

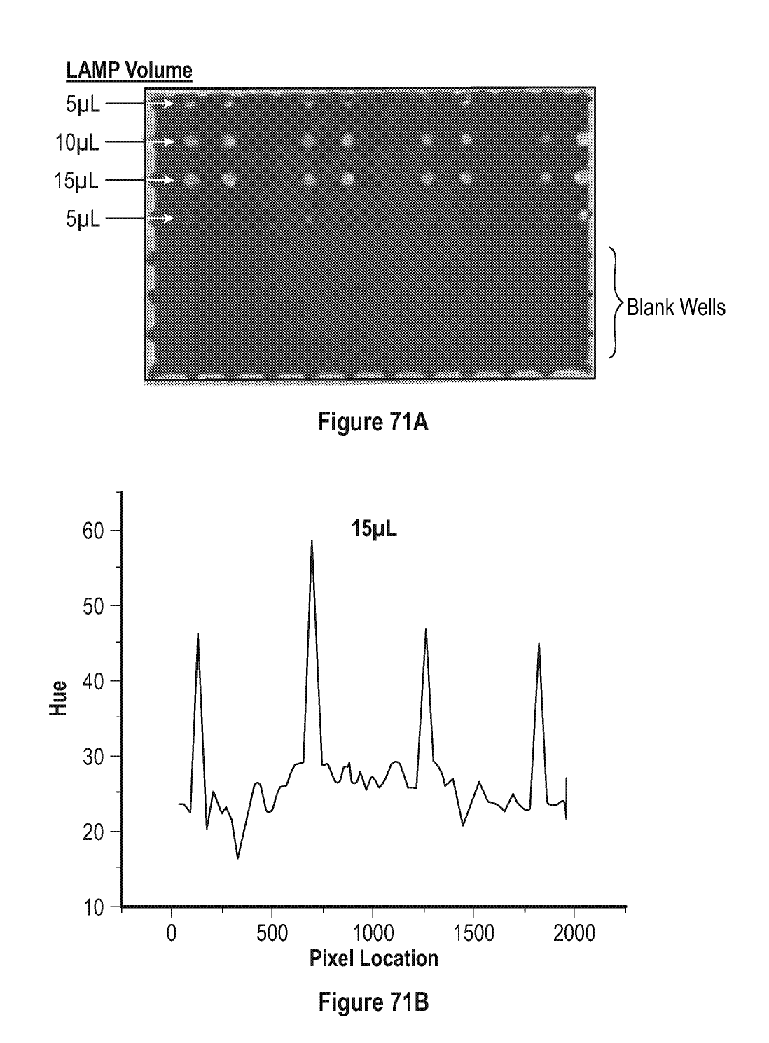

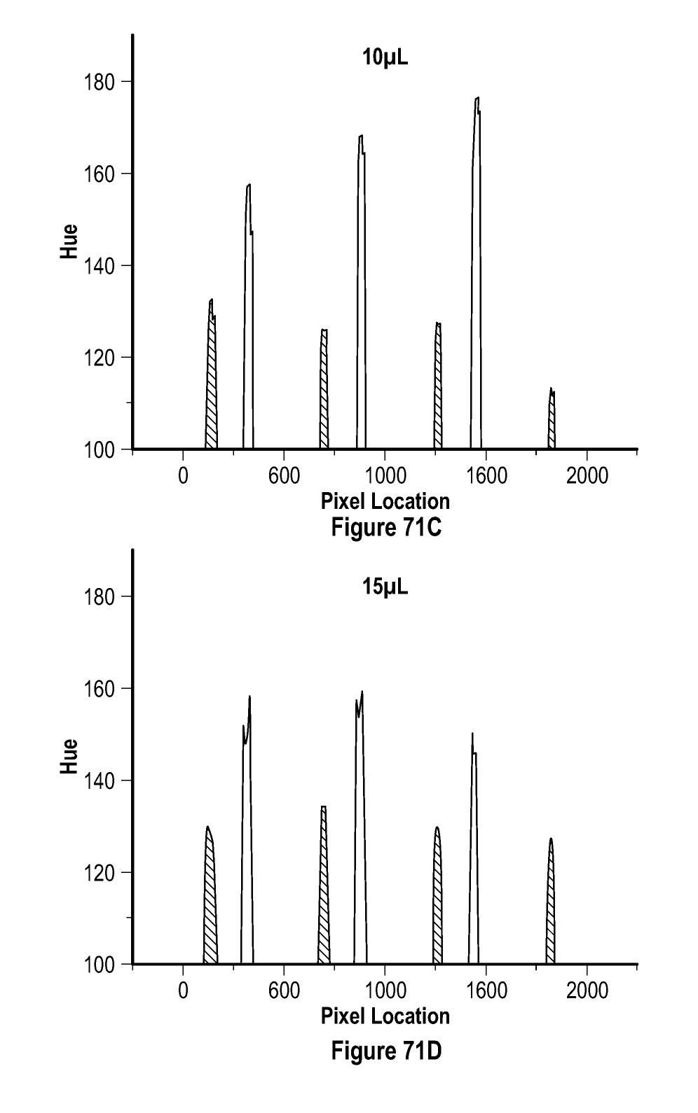

Sample Analysis. Conventional enzymatic and chemical testing methods are popular due to the rapid sampling time, inexpensive reagents, and clear visual colorimetric readout. Conventional LAMP amplifications with the precipitation of magnesium pyrophosphate become visually turbid, but could be difficult to decipher without fluorescence due to the low contrast of the turbidity relative to the background. Therefore, several methods have been developed for making LAMP reactions colorimetric. One of these methods uses an inexpensive hydroxynaphthol blue (BNB) dye which has been demonstrated to be compatible with LAMP reactions at an optimal concentration of 120 μM². A purple color, indicative of the chelation of Mg²⁺ by dNTPs², changes to a sky blue color as the concentration of free Mg²⁺ in solution depletes in solution with amplification (see FIG. 55). Sensitivity studies with this dye indicate that visual detection can occur with single copies of nucleic material which is useful for visual detection. To accommodate the maximum number of samples, colorimetric LAMP detection was adapted to a 96-well plate format. A rectangular case made of poly methyl methacrylate (PMMA) was made to fit a 96-well plate could fit directly in the top of the box (FIG. 56). A cell phone could then slide along the bottom of the box and take an image of all 96 wells from below. The color of the well will help determine what body fluids are present but it was unknown whether the color could be differentiated by cell phone analysis. To test this, a testing plate was filled with mock LAMP samples (15 μL volume) using either nuclease-free water or 8 nM MgSO₄ solution with 120 μM HNB to mimic the violet and sky blue colors before and after amplification, respectively. Using Imagej software, the hue for each well were plotted. All violet samples provided hue values between 170-200 whereas blue samples were below 150 (see FIGS. 60-61). Multiple photos of this plate provided identical hue profiles that clearly differentiates between positive and negative samples with a threshold hue of 150. Cell phone analysis of a second plate looked at the minimal volume required for analysis as well as the interpretation of blank wells. The plate was filled with three sets of 5, 10, and 15 μL positively and negatively mock LAMP samples that were separated by a blank well. The cell phone analysis could clearly distinguish samples down to 10 μL. Below this volume, the blue LAMP samples dropped out in the profiles (see FIGS. 71A-71D). Any blank wells in the 96-wells plate blended with baseline hue values and did not interfere with the optical detection analysis.

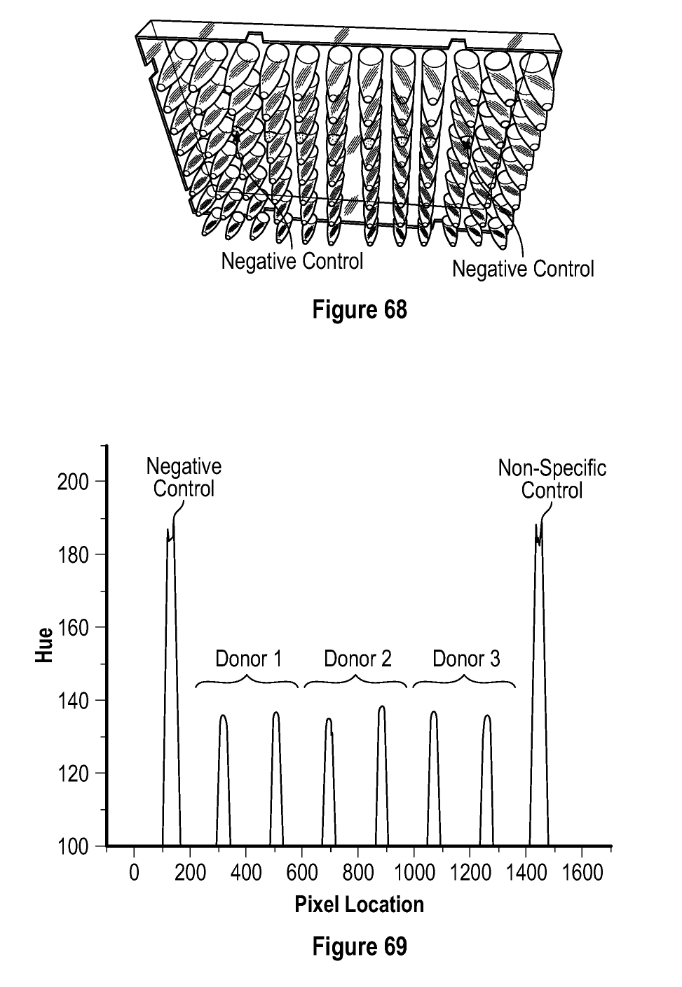

Challenging Samples. As SEMG1 is a prominent protein in semen, it was explored whether the semen LAMP assay was sensitive enough to identify azospermatic semen samples. To do this, duplicate samples from three separate donors were processed and amplified alongside a negative control (containing only TE buffer) and a non-specific control (1 ng human genomic DNA). All of the semen samples could be correctly identified within 30 minutes via cell phone imaging, as shown in FIGS. 68-69.

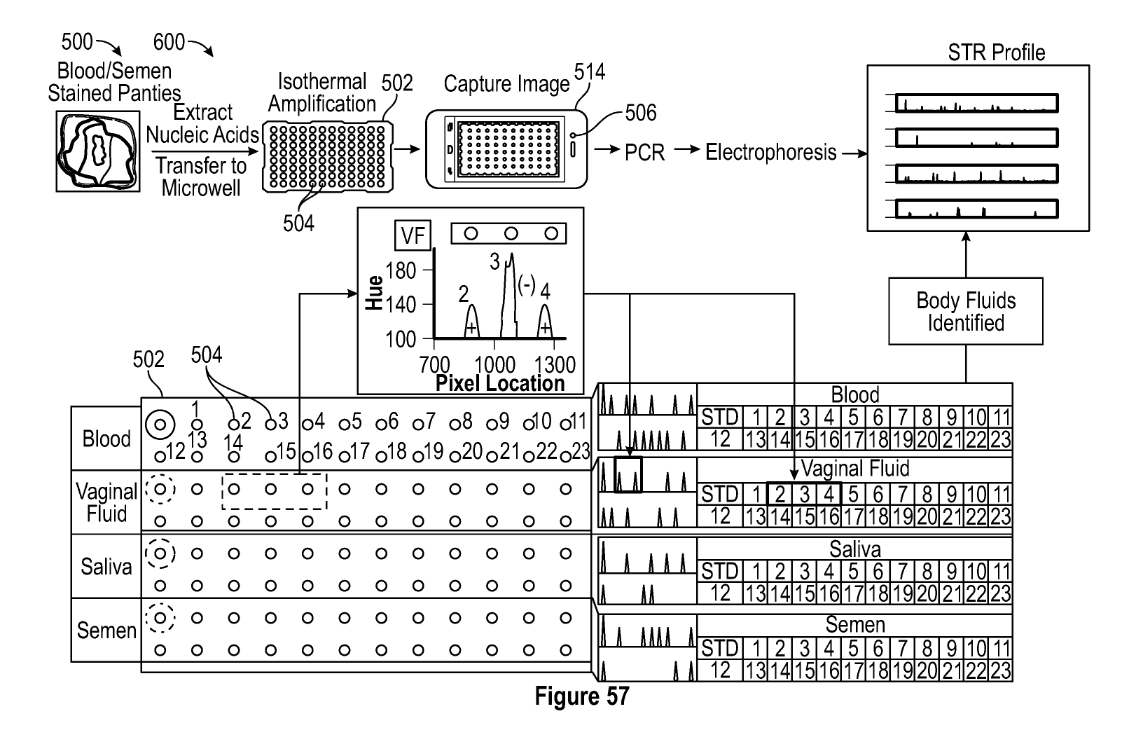

This technique was further challenged to see whether a series of 23 sample mixtures (containing between 1-3 body fluids) could be correctly identified via LAMP and optical detection. The 96-well plate was organized into 4-sections, with 2 consecutive rows assigned to each target body fluid. Negative controls were automatically placed at the beginning of the first row as an internal plate standard and to ensure that none of the reagents were contaminated. After a 30 minute amplification, the 96-well plate was placed in the PMMA box and captured via a smart phone. Using imagej software, the hue of unknown samples was compared to the negative controls. Only if the hue was below the threshold of 150 will a sample be identified for containing the target body fluid. Mapping a surface plot of the hue along the entire plate provided a digital readout of all the body fluids present in all 23 samples simultaneously (FIG. 57). Fortunately, all samples were correctly identified and did not provide any false negatives or positives.

Discussion LAMP provides many advantages over conventional PCR including high specificity, a large dynamic range, and single temperature amplification in a short 60 minute time frame. The high specificity of LAMP amplifications is derived from the four primers that recognize six regions along a target sequence. The ability for LAMP to discriminate between single nucleotides minimizes the chances of encountering any false positives. In addition, the dynamic range over five orders of magnitude allow detection of even trace samples which may be out of range for conventional enzymatic methods. Therefore, concerns regarding false positives and/or false negatives with qPCR can be eliminated with the adaptation to a LAMP protocol. As long as an mRNA target of interest is highly discriminatory among the body fluids of interest, there is no need for complex, statistical algorithms. The described LAMP method is also non-destructive and easily amendable to a simple workflow. Co-extraction of DNA and RNA prior to amplification reduces the analyst experimental time to obtain a forensic profile upon the confirmation of a body fluid via LAMP. In addition, a universal sampling method allowed the simultaneous identification of all four body fluids within 30 minutes which can provide examiners rapid and potentially critical contextual information to a given case. Embedding a metal chelating dye provided a colorimetric change that was easily captured with a camera, which avoided the use of expensive and complicated instrumentation. This colorimetric change also provided a digital ‘yes’ or ‘no’ response with an on-board negative control which delivered accurate detection of all body fluids. Unlike other body fluid panels, this method provides improved sensitivity from single copies of RNA and a clear sample-to-answer in <3 hours for 23 samples. Although this current method is only applicable to four body fluids, it could easily be extended to other body fluids including urine, sweat, and menstral blood.

### Conclusion

This LAMP body fluid identification panel has significant potential for accurate and rapid identification of blood, vaginal fluid, saliva, and semen in unknown samples. Four highly discriminatory mRNA markers were used to design LAMP assays amplifiable at the same temperature. Specificity studies validated the accurate identification of each target body fluid which was reproducible between multiple users. The elegance of this approach is that it does not involve thermocycling and uses a simple dye for colorimetric read-out and a smart phone as a detector. Relative to existing methods, this provides an accelerated sample-to-answer method for mRNA with high specificity and sensitivity (single copies of RNA) and unparalleled bandwidth (5 fluids) for body fluid ID. This method was challenged with dry stains on denim material and azospermatic samples which both performed remarkedly well and could be successfully detected. A blind study also validated the efficacy of the method and demonstrated that the method can be used by individuals who are not highly trained. Future efforts will focus on automation of this system, eliminating the need for separate thermocyclers or heat plates. The number of target body fluids will also expand to accommodate all forensically-relevant samples. Image analysis will also be improved to provide a simple table display of all body fluids contained in a given sample.

### Example 6

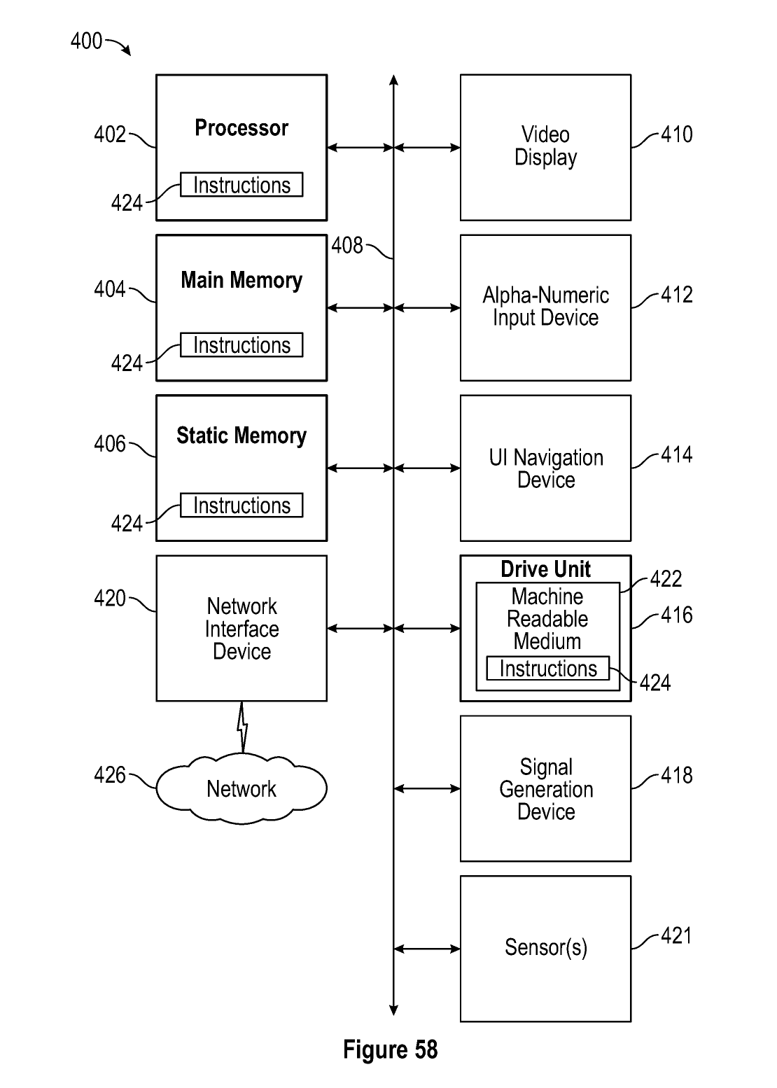

FIG. 58 illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Thus, the system and method provide for the development of a universal protocol with unique primer sequences for specific detection, a high throughput process, with capability of analyzing, e.g., 96, sample wells simultaneously. Cell phone detection captures a RGB image and converts images to look specifically at differences in colors by hue grayscale analysis. Therefore, the system and method provide a rapid, simple, and cost-effect method for identification of body fluids.

Examples 3 to 6 and the supporting FIGS. 54-71D describe various methods and systems including a method and system (show in FIG. 57) to simultaneously detect one or more target messenger RNA sequences in a plurality of samples. One exemplary method 500 is shown in FIG. 57. The method 500 can include providing a platform 502 (shown in FIGS. 55-57) configured to receive the plurality of samples 504 (FIGS. 56 and 57) therein. The method 500 can insert a dye in each of the plurality of samples 504 for colorimeteric read-out as previously described and shown in FIGS. 57, 69 and 71A-71D. The method 500 can subject the plurality of samples 504 and dye to a single temperature isothermal amplification reaction and can include imaging the platform with a camera 506 (FIG. 57) to collect image data. The method 500 can also perform imaging analysis on the image data using a machine (e.g., the machine 400 and/or the cell phone shown in FIGS. 55 and 57) to determine hue values of the plurality of samples. The method 500 analyzes the hue values to determine if one or more of the samples contains the one or more target messenger RNA sequences as previously described. The one or more target messenger RNA sequences can be from human bodily fluid that comprises one or more of blood, vaginal fluid, semen and saliva.

As shown in FIG. 56, the platform 502 comprise a plate 508 with 96 individually separated sample wells 510. As shown in FIG. 55, according to one example the platform 502 can be disposed as a wall 502A within an imaging booth 512. The camera 506 (FIG. 57) is part of a handheld electronic device 514 (e.g., a smartphone) that can be disposed at least partially within the imaging booth 512 as shown in FIG. 55. The handheld electronic device 514 comprises the machine upon which the image analysis and analyzing of the hue values can be performed as described above.

According to one embodiment, performing imaging analysis and analyzing according to the method 500 can include one or more of converting a color space to an 8-bit grayscale and linearly scaling pixels between 0-255, extending a linear line across each horizontal row of the plurality of samples, and accessing whether the hue valves of the plurality of samples exceeded a threshold hue value.

FIGS. 55-58 show components that also can be used as part of a system 600 (FIG. 57) including the imaging booth 512 and the handheld electronic device 514. As shown in FIG. 55, the handheld electronic device can be configured to be at least partially received in the imaging booth 512. The handheld electronic device can have the camera 506 configured to image a portion of the imaging booth 512 and can have computer (e.g. machine 400) including at least one processor (e.g., processor 402) and a memory device (e.g. memory 404, 406), the memory device including instructions (e.g., instructions 424) that, when executed by the at least one processor, cause the computer to access image data of the portion of the imaging booth imaged by the camera 506. The portion (e.g. wall 502a) of the imaging booth 512 configured to receive the plurality of samples 504 and dye therein. The system can perform analysis on the image data including analyzing the hue values of the plurality of samples and dye to determine if one or more of the samples contains a target messenger RNA sequence.

### REFERENCES

Harbison, Research and Reports in Forensic Medical Science, 2016, 2016:6, 11-23.
Butler, Advanced Topics in Forensic DNA Typing, Academic Press, 2011.
Casey and J. Price, Forensic Science International, 2010, 194, 67-71.
Johnston, J. Newman and R. Frappier, Canadian Society of Forensic Science Journal, 2003, 36, 173-183.
Diamandis and H. Yu, The Lancet, 1995, 345, 1186.
Nagar and K. K. Sharma, Indian Journal of Clinical Biochemistry, 2012, 27, 375-378.
Ricci, I. Carboni and F. Torricelli, Journal of forensic sciences, 2014, 59, 1410-1412.
Zubakov, E. Hanekamp, M. Kokshoorn, W. van IJcken and M. Kayser, International journal of legal medicine, 2008, 122, 135-142.
Setzer, J. Juusola and J. Ballantyne, Journal of forensic sciences, 2008, 53, 296-305.
Park, S.-M. Park, J.-H. Kim, H.-C. Lee, S.-H. Lee, K.-M. Woo and S.-Y. Kim, Genomics Inform, 2013, 11, 277-281.
Richard, K. A. Harper, R. L. Craig, A. J. Onorato, J. M. Robertson and J. Donfack, Forensic Science International-Genetics, 2012, 6, 452-460.
Haas, B. Klesser, C. Maake, W. Baer and A. Kratzer, Forensic Science International-Genetics, 2009, 3, 80-88.
Danaher, R. L. White, E. K. Hanson and J. Ballantyne, Forensic Science International: Genetics, 2015, 14, 18-30.
Su, C.-Y. Li, J. C.-I. Lee, D.-D. Ji, S.-Y. Li, B. Daniel, D. Syndercombe-Court, A. Linacre and H.-M. Hsieh, Forensic Science Medicine and Pathology, 2015, 11, 208-215.
Norris, Doctoral Dissertation, University of Virginia, 2008.
Kimes and M. Tamir, Crime Laboratory Digest, 1985, 12, 32-33.
Fan, M. Yan, P. Du, C. Chen and B. Kan, Foodborne pathogens and disease, 2015, 12, 778-786.
Niessen, J. Luo, C. Denschlag and R. F. Vogel, Food microbiology, 2013, 36, 191-206.
Hagan, C. R. Reedy, J. M. Bienvenue, A. H. Dewald and J. P. Landers, Analyst, 2011, 136, 1928-1937.
Kaneko, T. Iida, K. Aoki, S. Ohno and T. Suzutani, Journal of clinical microbiology, 2005, 43, 3290-3296.
Nakanishi, T. Ohmori, M. Hara, A. Takada, H. Shojo, N. Adachi and K. Saito, Journal of forensic sciences, 2011, 56.
Fischbach, N. C. Xander, M. Frohme and J. F. Gloekler, Biotechniques, 2015, 58, 189-194.
Tomita, Y. Mori, H. Kanda and T. Notomi, Nat. Protocols, 2008, 3, 877-882.
Hadersdorfer, M. Neumuller, D. Treutter and T. C. Fischer, Annals of Applied Biology, 2011, 159, 456-466.
Soli, M. Kas, T. Maure, M. Umezaki, A. Morita, P. M. Siba, A. R. Greenhill and P. F. Horwood, Diagnostic Microbiology and Infectious Disease, 2013, 77, 321-323.
Goto, E. Honda, A. Ogura, A. Nomoto and K. I. Hanaki, Biotechniques, 2009, 46, 167-+.
Luo, K. Nie, M. J. Yang, M. Wang, J. Li, C. Zhang, H. T. Liu and X. J. Ma, Journal of Clinical Microbiology, 2011, 49, 3545-3550.
Ghosh, A. Nagavardhini, A. Sengupta and M. Sharma, Bmc Research Notes, 2015, 8.
Iwasaki, T. Yonekawa, K. Otsuka, W. Suzuki, K. Nagamine, T. Hase, K.-I. Tatsumi, T. Horigome, T. Notomi and H. Kanda, Genome Letters, 2003, 2, 119-126.
James, S. L. N. N., Backlog of Sexual Assault Evidence: In Brief, C. R. Service, Editor. 2015.
Danaher, P., et al., Facile semi-automated forensic body fluid identification by multiplex solution hybridization of NanoString® barcode probes to specific mRNA targets. Forensic Science International: Genetics, 2015. 14: p. 18-30.
Su, C.-W., et al., A novel application of real-time RT-LAMP for body fluid identification: using HBB detection as the model. Forensic Science Medicine and Pathology, 2015. 11(2): p. 208-215.
Burgess, A. W., D. M. Slattery, and P. A. Herlihy, Military sexual trauma: A silent syndrome. Journal of psychosocial nursing and mental health services, 2013. 51(2): p. 20-26.
Dou, M., et al., A Versatile PDMS/Paper Hybrid Microfluidic Platform for Sensitive Infectious Disease Diagnosis. Analytical Chemistry, 2014. 86(15): p. 7978-7986.
Notomi, T.; Okayama, H.; Masubuchi, H.; et al. Loop-mediated isothermal amplification Of DNA. Nucleic Acids Research. 2000. 28. 12.
Butler, J., Advanced Topics in Forensic DNA Typing. 2011: Academic Press.
Cho, Y. K., et al., One-step pathogen specific DNA extraction from whole blood on a centrifugal microfluidic device. Lab on a Chip, 2007. 7(5): p. 565-573.
Gan, W. P., et al., A filter paper-based microdevice for low-cost, rapid, and automated DNA extraction and amplification from diverse sample types. Lab on a Chip, 2014. 14(19): p. 3719-3728.
Karle, M., et al., Continuous microfluidic DNA extraction using phase-transfer magnetophoresis. Lab on a Chip, 2010. 10(23): p. 3284-3290.
Chung, Y. C., et al., Microfluidic chip for high efficiency DNA extraction. Lab on a Chip, 2004. 4(2): p. 141-147.
Lounsbury, J. A., et al., From sample to PCR product in under 45 minutes: a polymeric integrated microdevice for clinical and forensic DNA analysis. Lab on a Chip, 2013. 13(7): p. 1384-1393.
Duarte, G. R. M., et al., Dynamic solid phase DNA extraction and PCR amplification in polyester-toner based microchip. Analytical Chemistry, 2011. 83(13): p. 5182-5189.
Hopwood, A. J., et al., Integrated Microfluidic System for Rapid Forensic DNA Analysis: Sample Collection to DNA Profile. Analytical Chemistry, 2010. 82: p. 6991-6999.
Azimi, S., et al., A magnetic bead-based DNA extraction and purification microfluidic device. Microfluidics and Nanofluidics, 2011. 11(2): p. 157-165.
International Patent Application No. PCT/US2013/074152 entitled “FREQUENCY-BASED FILTERING OF MECHANICAL ACTUATION USING FLUIDIC DEVICE,” filed Dec. 10, 2013.
U.S. patent application Ser. No. 13/849,691 entitled “Electrostatic Actuation for Management of Flow in Micro Total Analysis Systems and Related Method Thereof,” filed Mar. 25, 2013.
U.S. patent application Ser. No. 13/294,908 entitled “Electrostatic Actuation for Management of Flow in Micro-Total Analysis Systems (u-TAS) and Related Method Thereof,” filed Nov. 11, 2011; U.S. Pat. No. 8,403,294, issued Mar. 26, 2013.
U.S. patent application Ser. No. 11/665,326 entitled “Electrostatic Actuation for Management of Flow in Micro Total Analysis Systems and Related Method Thereof,” filed Apr. 13, 2007; U.S. Pat. No. 8,056,881, issued Nov. 15, 2011
International Patent Application No. US2005/036675 entitled “Electrostatic Actuation for Management of Flow in Micro Total Analysis Systems and Related Method Thereof,” filed Oct. 13, 2005.
U.S. patent application Ser. No. 13/699,983 entitled “METHOD FOR DETECTING NUCLEIC ACIDS BASED ON AGGREGATE FORMATION,” filed Nov. 26, 2012;
U.S. Patent Application Publication No. 2013/0203045, Aug. 8, 2013.
International Patent Application No. PCT/US2011/038166 entitled “METHOD FOR DETECTING NUCLEIC ACIDS BASED ON AGGREGATE FORMATION,” filed May 26, 2011.
U.S. patent application Ser. No. 13/474,420 entitled “Passive Components for Micro-Fluidic Flow Profile Shaping and Related Method Thereof,” filed May 17, 2012; U.S. Patent Application Publication No. 2012/0222747, Sep. 6, 2012.
U.S. patent application Ser. No. 12/064,557 entitled “Passive Components for Micro-Fluidic Flow Profile Shaping and Related Method Thereof,” filed Feb. 22, 2008; U.S. Pat. No. 8,220,493, issued Jul. 17, 2012.
International Patent Application No. PCT/US2006/032717 entitled “Passive Components for Micro-Fluidic Flow Profile Shaping and Related Method Thereof,” filed Aug. 23, 2006.
International Patent Application No. PCT/US2012/036105 entitled “METHOD AND SYSTEM FOR HIGH THROUGHPUT OPTICAL AND LABEL FREE DETECTION OF ANALYTES,” filed May 2, 2012.
U.S. patent application Ser. No. 13/505,406 entitled “VERSATILE, VISIBLE METHOD FOR DETECTING POLYMERIC ANALYTES,” filed May 1, 2012; U.S. Patent Application Publication No. 2013/0084565, Apr. 4, 2013.
U.S. patent application Ser. No. 13/116,659 entitled “METHOD FOR DETECTING NUCLEATED CELLS,” filed May 26, 2011.
International Patent Application No. PCT/US2010/002883 entitled “VERSATILE, VISIBLE METHOD FOR DETECTING POLYMERIC ANALYTES,” filed Nov. 3, 2010.
U.S. patent application Ser. No. 12/879,810 entitled “DETECTION OF POLYMERIC ANALYTES,” filed Sep. 10, 2010; U.S. Patent Application Publication No. 2011/0070660, Mar. 24, 2011.
International Patent Application No. PCT/US2009/036983 entitled “DETECTION OF POLYMERIC ANALYTES,” filed Mar. 12, 2009.
U.S. patent application Ser. No. 12/892,618 entitled “DNA EXTRACTION USING A PHOTO-POLYMERIZED MONOLITH IN A CAPILLARY,” filed Sep. 28, 2010;
U.S. Patent Application Publication No. 2011/0086181, Apr. 14, 2011.
U.S. patent application Ser. No. 11/885,181 entitled “Grafted Photo-Polymerized Monolithic Column,” filed Aug. 28, 2007; U.S. Pat. No. 7,815,802, issued Oct. 19, 2010.
International Patent Application No. US2006/006845 entitled “DNA Extraction Using a Photo-Polymerized Monolith in a Capillary,” filed Feb. 28, 2006. (01106-03)
U.S. patent application Ser. No. 12/090,233 entitled “Integrated Microfluidic Analysis Systems,” filed Apr. 14, 2008.
International Patent Application No. US2006/039809 entitled “Integrated Microfluidic Analysis Systems,” filed Oct. 12, 2006.
U.S. patent application Ser. No. 12/089,320 entitled “Microchip-based Acoustic Trapping or Capture of Cells for Forensic Analysis and Related Method Thereof,” filed Apr. 4, 2008.
International Patent Application No. PCT/US2006/038943 entitled “Microchip-based Acoustic Trapping or Capture of Cells for Forensic Analysis and Related Method Thereof,” filed Oct. 4, 2006.
U.S. patent application Ser. No. 11/989,794 entitled “Microdevices for Chemical Sensing and Chemical Actuation,” filed Jan. 31, 2008; U.S. Pat. No. 8,343,755, issued Jan. 1, 2013.
International Patent Application No. PCT/US2006/030127 entitled “Microdevices for Chemical Sensing and Chemical Actuation,” filed Aug. 1, 2006.
U.S. patent application Ser. No. 11/793,428 entitled “Use of Microwaves for Thermal and Non-Thermal Applications in Micro and Nanoscale Devices,” filed Jan. 7, 2008; U.S. Patent Application Publication No. 2008/0277387, Nov. 13, 2008.)
International Patent Application No. US2005/046756 entitled “The Use of Microwaves for Thermal and Non-Thermal Applications in Micro and Nanoscale Devices,” filed Dec. 22, 2005.
International Patent Application No. PCT/US2007/088662 entitled “Non-Contact Thermal Control of Small Volume and Related Apparatus Thereof,” filed Dec. 21, 2007.
International Patent Application No. PCT/US2007/083964 entitled “DNA Purification in a Multi-Stage, Multi-Phase Microchip,” filed Nov. 7, 2007.
U.S. patent application Ser. No. 11/884,351 entitled “Nucleic Acid Isolation Methods and Materials and Devices Thereof,” filed Aug. 15, 2007.
International Patent Application No. PCT/US2006/005241 entitled “Nucleic Acid Isolation Methods and Materials and Devices Thereof,” filed Feb. 15, 2006.
U.S. patent application Ser. No. 11/664,297 entitled “Localized Control of Thermal Properties on Microdevices and Applications Thereof,” filed Mar. 19, 2007.
International Patent Application No. US2005/034674 entitled “Localized Control of Thermal Properties on Microdevices and Applications Thereof,” filed Sep. 29, 2005.
U.S. patent application Ser. No. 10/535,926 entitled “Isolation of Sperm Cells from Other Biological Materials Using Microfabricated Devices and Related Methods Thereof,” filed May 23, 2005.
International Patent Application No. US2003/037205 entitled “Isolation of Sperm Cells from Other Biological Materials Using Microfabricated Devices and Related Methods Thereof,” filed Nov. 20, 2003.
U.S. patent application Ser. No. 10/530,728 entitled “Methods and Systems for Multiplexing IR-Mediated Heating on a Microchip,” filed Apr. 8, 2005.
International Patent Application No. US2003/031806 entitled “Methods and Systems for Multiplexing IR Mediated Heating on a Microchip,” filed Oct. 8, 2003.
International Patent Application No. US2005/005490 entitled “Method and System for Eluting Cells,” filed Feb. 22, 2005
U.S. patent application Ser. No. 10/520,763 entitled “Hybrid Polymers for Functional Tuning of Microfluidic Device Surfaces,” filed Jan. 10, 2005; U.S. Patent No.
International Patent Application No. US2003/022162 entitled “Hybrid Polymers for Functional Tuning of Microfluidic Device Surfaces,” filed Jul. 15, 2003.
U.S. patent application Ser. No. 10/517,980 entitled “Apparatus and Method for Purification of Nucleic Acids,” filed Dec. 13, 2004; U.S. Pat. No. 7,534,623, issued May 19, 2009
International Patent Application No. US2003/018403 entitled “Apparatus and Method or Purification of Nucleic Acids,” filed Jun. 11, 2003.
International Patent Application No. US2004/001276 entitled “Method for Microchip and Capillary Detection of Proteins in the Sub-μG/ML Range,” filed Jan. 20, 2004.
U.S. patent application Ser. No. 10/664,064 entitled “Remote Temperature Sensing of Small Volume and Related Apparatus Thereof,” filed Sep. 17, 2003.
International Patent Application No. US2003/029249 entitled “Remote Temperature Sensing of Small Volume and Related Apparatus Thereof,” filed Sep. 17, 2003.
U.S. patent application Ser. No. 10/432,141 entitled “Method for Orthogonal Analyte Stacking/Injection Systems in Electrophoresis,” filed May 16, 2003.
International Patent Application No. US01/43259 entitled “Method For Orthogonal Analyte Stacking/Injection Systems In Electrophoresis,” filed Nov. 19, 2001.
   Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions may or may not be provided on the aforementioned figures, the device may constitute various sizes, dimensions, contours, rigidity, shapes, flexibility and materials as it pertains to the components or portions of components of the device, and therefore may be varied and utilized as desired or required.

It should be appreciated that the device and related components discussed herein may take on all shapes along the entire continual geometric spectrum of manipulation of x, y and z planes to provide and meet the structural demands and operational requirements. Moreover, locations and alignments of the various components may vary as desired or required.

### Further Notes and Examples

Example 1 is a method to detect a target sequence in a sample, the method can comprise: providing one or more aliquots of an amplification reaction specific for amplifying the target sequence in a sample; contacting the aliquot, magnetic beads and an amount of isolated nucleic acid of greater than about 1 is missing parent: 5 kb in length, optionally under chaotropic conditions, thereby providing a mixture; subjecting the mixture to conditions that allow for aggregation of the beads, wherein the absence of aggregation under the conditions is indicative of the presence of amplified target sequence; and detecting the presence or amount of aggregation.

In Example 2, the subject matter of Example 1 optionally includes wherein the target sequence is from a pathogen.

In Example 3, the subject matter of Example 2 optionally includes wherein the pathogen is a bacterium, virus or parasite.

In Example 4, the subject matter of any one or more of Examples 1-3 optionally include 10 kb in length.

In Example 5, the subject matter of any one or more of Examples 1-4 optionally include wherein the conditions to allow aggregation are a rotating magnetic field, acoustic energy or vibration.

In Example 6, the subject matter of any one or more of Examples 1-5 optionally include wherein the aliquot is contacted with the beads before contact with the isolated nucleic acid.

In Example 7, the subject matter of any one or more of Examples 1-6 optionally include wherein the aggregation is not sequence-specific.

In Example 8, the subject matter of any one or more of Examples 1-7 optionally include wherein the magnetic beads are coated with silica.

In Example 9, the subject matter of any one or more of Examples 1-8 optionally include wherein the amount of aggregation is monitored over time for the amplification reaction.

In Example 10, the subject matter of any one or more of Examples 1-9 optionally include wherein the sample is a blood sample, urine sample, plasma or serum sample, nasal swab sample, or a cerebrospinal fluid sample.

In Example 11, the subject matter of any one or more of Examples 1-10 optionally include wherein the sample comprises cells.

In Example 12, the subject matter of Example 11 optionally includes wherein the sample comprises human cells.

In Example 13, the subject matter of any one or more of Examples 1-12 optionally include wherein the sample is a tissue biopsy.

In Example 14, the subject matter of any one or more of Examples 1-13 optionally include wherein aggregation is detected using a system that contains a camera and optionally analyzes images from the camera.

In Example 15, the subject matter of Example 14 optionally includes wherein the system comprises a cell phone.

In Example 16, the subject matter of any one or more of Examples 1-15 optionally include wherein the amplification reaction is a loop mediated isothermal amplification reaction. In Example 17, the subject matter of any one or more of Examples 1-16 optionally include wherein the amplification reaction is on a chip.

In Example 18, the subject matter of any one or more of Examples 1-17 optionally include wherein the beads and the isolated nucleic acid are added to the chip.

In Example 19, the subject matter of any one or more of Examples 1-18 optionally include wherein the isolated nucleic acid comprises genomic DNA.

In Example 20, the subject matter of Example 19 optionally includes wherein the isolated nucleic acid is human genomic DNA.

Example 21 is a device configured to isolate nucleic acid through application of a centrifugal force thereto, the device can comprise: a mixing chamber selectively connected to one or more of a first wash buffer and an elution buffer and configured to receive a sample therein; a waste chamber selectively connected to the mixing chamber by one of a first stop valve and a first siphon valve, the first stop valve configured to be forced open after the device is rotated at a first rotational speed with the first wash buffer disposed in the mixing chamber to allow for passage of a waste from the mixing chamber to the waste chamber; and a nucleic acid recovery chamber selectively connected to the mixing chamber by a second stop valve, the second stop valve configured to be forced open after the device is rotated at a second rotational speed with the elution buffer disposed in the mixing chamber to allow for passage of the nucleic acid from the mixing chamber to the nucleic acid recovery chamber.

In Example 22, the subject matter of Example 21 optionally includes wherein the first stop valve is configured to be forced open at a first burst pressure to allow for passage of the waste from the mixing chamber to the waste chamber, wherein the second stop valve is configured to be forced open at a second burst pressure that differs from the first burst pressure to allow for passage of the nucleic acid from the mixing chamber to the nucleic acid recovery chamber, and wherein the second burst pressure is lower than the first burst pressure.

In Example 23, the subject matter of any one or more of Examples 21-22 optionally include at least one elution chamber configured to contain the elution buffer and selectively connected to the mixing chamber by a first hydrophobic valve; a first wash chamber configured to contain the first wash buffer and selectively connected to the mixing chamber is by a second siphon valve; and a second wash chamber configured to contain a second wash buffer and selectively connected to the mixing chamber by a second hydrophobic valve.

In Example 24, the subject matter of Example 23 optionally includes wherein the elution buffer comprises a chaotropic solution to bind the nucleic acid to beads, wherein the first wash buffer comprises an alcohol solution, and wherein the second wash buffer comprises a low salt buffer.

In Example 25, the subject matter of any one or more of Examples 21-24 optionally include wherein one or more of the first stop valve and the second stop valve are formed of a plurality of polyethylene terephthalate layers including a first layer configured to define a valve seat that comprises a physical barrier separating an incoming channel from an outgoing channel of the valve.

In Example 26, the subject matter of Example 25 optionally includes wherein the plurality of polyethylene terephthalate layers includes a second layer configured to form at least a portion of the incoming channel and the outgoing channel of the valve.

In Example 27, the subject matter of Example 26 optionally includes a double-sided adhesive configured to adhere to the first layer; and a second adhesive comprising a pressure-sensitive adhesive that is couple to the double-sided adhesive.

In Example 28, the subject matter of any one or more of Examples 21-27 optionally include wherein the first siphon valve is configured to allow access to the waste within the waste chamber after an initial binding, and during an alcohol wash, and is further configured to prevent the elution buffer from entering the waste chamber.

In Example 29, the subject matter of any one or more of Examples 21-28 optionally include wherein the second rotational speed that differs from the first rotational speed.

Example 30 is a disc including the device of any one of Examples 21 to 29, wherein the device can comprise a plurality of devices that are circumferentially distributed around the disc such that the centrifugal force can be applied on the plurality of devices simultaneously.

Example 31 is a second device disposed adjacent to and spaced apart from the disc of Example 30 by a variable height, the second device having one or more magnets mounted thereto, the one or more magnets are configured to exert a magnetic force on particles within the mixing chamber, and wherein the disc is configured to be rotated both clockwise and counter-clockwise relative to the one or more magnets to change a direction of the magnetic force on the particles to mix the particles within the mixing chamber.

Example 32 is a method of extracting nucleic acid using a device constructed of plurality of polyethylene terephthalate layers, the method can comprise: loading a sample into a mixing chamber formed by one or more of the plurality of polyethylene terephthalate layers, the mixing chamber connected to a waste chamber by one of a first stop valve and a first siphon valve, and the mixing chamber selectively connected to a nucleic acid recovery chamber by a second stop valve; rotating the device at a first rotational speed to release a first wash into the mixing chamber and subsequently through the first stop valve or the first siphon valve into the waste chamber; rotating the device at a second rotational speed to release an elution buffer to the mixing chamber to mix with the sample and magnetic particles to release the nucleic acid; and rotating the device at a third rotational speed to pass the nucleic acid through the second stop valve into the nucleic acid recovery chamber.

In Example 33, the subject matter of Example 32 optionally includes sequent to rotating the device to release the first wash, opening the first stop valve to the waste chamber; and sequent to rotating the device to release an elution buffer to the mixing chamber, closing the first stop valve to the waste chamber.

In Example 34, the subject matter of any one or more of Examples 32-33 optionally include rotating the device at a fourth rotational speed to release a second wash into the mixing chamber and subsequently through the first stop valve or the first siphon valve into the waste chamber.

In Example 35, the subject matter of Example 34 optionally includes wherein the first rotational speed, the second rotational speed, the third rotational speed, and the fourth rotational speed all differ.

In Example 36, the subject matter of any one or more of Examples 32-35 optionally include applying a magnetic force on the magnetic particles within the mixing chamber, and wherein the device is configured to be rotated both clockwise and counter-clockwise to change a direction of the magnetic force on the particles to mix the magnetic particles within the mixing chamber.

In Example 37, the subject matter of Example 36 optionally includes varying the intensity of the magnetic force on the magnetic particles within the mixing chamber.

In Example 38, the subject matter of any one or more of Examples 32-37 optionally include wherein the rotating the device at the third rotational speed to pass the nucleic acid through the second stop valve comprises forcing the second stop valve open by exceeding a burst pressure thereof.

Example 39 is an apparatus configured to carry a centrifugal microfluidic device to prepare a sample for analysis, the apparatus can comprise: a disc mounted to the apparatus, the disc constructed of plurality of polyethylene terephthalate layers configured to form a plurality of devices, each device includes: a mixing chamber selectively connected to one or more of a first wash buffer and an elution buffer and configured to receive a sample therein, a waste chamber selectively connected to the mixing chamber by one of a first stop valve and a first siphon valve, the first stop valve configured to be forced open after the device is rotated at a first rotational speed with the first wash buffer disposed in the mixing chamber to allow for passage of a waste from the mixing chamber to the waste chamber, and a nucleic acid recovery chamber selectively connected to the mixing chamber by a second stop valve, the second stop valve configured to be forced open after the device is rotated at a second rotational speed with the elution buffer disposed in the mixing chamber to allow for passage of the nucleic acid from the mixing chamber to the nucleic acid recovery chamber, a first motor configured to rotate the disc; a second device disposed a distance apart from the disc, the second device having one or more magnets mounted thereto, the one or more magnets are configured to exert a magnetic force on particles within the mixing chamber, and wherein the disc is configured to be rotated both clockwise and counter-clockwise relative to the one or more magnets to change a direction of the magnetic force on the particles to mix the particles within the mixing chamber; and a second motor configured to adjust the distance apart the disc is disposed from the second device.

In Example 40, the subject matter of Example 39 optionally includes wherein the device further comprises: at least one elution chamber configured to contain the elution buffer and selectively connected to the mixing chamber by a first hydrophobic valve; a first wash chamber configured to contain the first wash buffer and selectively connected to the mixing chamber is by a second hydrophobic valve; and a second wash chamber configured to contain a second wash buffer and selectively connected to the mixing chamber by a second siphon valve.

Example 41 is a method to simultaneously detect one or more target messenger RNA sequences in a plurality of samples, comprising: providing a platform configured to receive the plurality of samples therein; inserting a dye in each of the plurality of samples for colorimeteric read-out; subjecting the plurality of samples and dye to a single temperature isothermal amplification reaction; imaging the platform with a camera to collect image data; and performing imaging analysis on the image data using a machine to determine hue values of the plurality of samples; and analyzing the hue values to determine if one or more of the samples contains the one or more target messenger RNA sequences.

In Example 42, the subject matter of Example 41 optionally includes individually separated sample wells.

In Example 43, the subject matter of any one or more of Examples 41-42 optionally include wherein the one or more target messenger RNA sequences is from human bodily fluid.

In Example 44, the subject matter of Example 43 optionally includes wherein the human bodily fluid comprises one or more of blood, vaginal fluid, semen and saliva.

In Example 45, the subject matter of any one or more of Examples 41-44 optionally include disposing the platform as a wall within an imaging booth, and wherein the camera is part of a handheld electronic device disposed at least partially within the imaging booth and the handheld electronic device comprises the machine upon which the image analysis and analyzing of the hue values is performed.

In Example 46, the subject matter of any one or more of Examples 41-45 optionally include wherein performing imaging analysis and analyzing includes: converting a color space to an 8-bit grayscale and linearly scaling pixels between 0-255; extending a linear line across each horizontal row of the plurality of samples; and accessing whether the hue valves of the plurality of samples exceeded a threshold hue value.

Example 47 is a system can comprise: an imaging booth; a handheld electronic device configured to be at least partially received in the imaging booth, the handheld electronic device having a camera configured to image a portion of the imaging booth and having computer including at least one processor and a memory device, the memory device including instructions that, when executed by the at least one processor, cause the computer to: access image data of the portion of the imaging booth imaged by the camera, the portion of the imaging booth configured to receive the plurality of samples and dye therein; and perform analysis on the image data including analyzing the hue values of the plurality of samples and dye to determine if one or more of the samples contains a target messenger RNA sequence.

In Example 48, the subject matter of Example 47 optionally includes individually separated sample wells.

In Example 49, the subject matter of any one or more of Examples 47-48 optionally include wherein the target messenger RNA sequence is from human bodily fluid and comprises one or more of blood, vaginal fluid, semen and saliva.

In Example 50, the subject matter of any one or more of Examples 47-49 optionally include wherein the portion of the imaging booth being comprises a wall of the imaging booth.

In Example 51, the subject matter of any one or more of Examples 47-50 wherein optionally analysis includes conversion of a color space to an 8-bit grayscale and linearly scale pixels between 0-255, extend a linear line across each horizontal row of the plurality of samples, and access whether the hue valves of the plurality of samples exceeded a threshold hue value.

1. A method to detect a target sequence in a sample, comprising:
providing one or more aliquots of an amplification reaction specific for amplifying the target sequence in a sample;
contacting the aliquot, magnetic beads and an amount of isolated nucleic acid of greater than about 5 kb in length, optionally under chaotropic conditions, thereby providing a mixture;
subjecting the mixture to conditions that allow for aggregation of the beads, wherein the absence of aggregation under the conditions is indicative of the presence of amplified target sequence; and
detecting the presence or amount of aggregation.

2. The method of claim 1 wherein the target sequence is from a pathogen.

3. The method of claim 2 wherein the pathogen is a bacterium, virus or parasite.

4. The method of claim 1 wherein the isolated nucleic acid is greater than about 10 kb in length.

5. The method of claim 1 wherein the conditions to allow aggregation are a rotating magnetic field, acoustic energy or vibration.

6. The method of claim 1 wherein the aliquot is contacted with the beads before contact with the isolated nucleic acid.

7. The method of claim 1 wherein the aggregation is not sequence-specific.

8. The method of claim 1 wherein the magnetic beads are coated with silica.

9. The method of claim 1 wherein the amount of aggregation is monitored over time for the amplification reaction.

10. The method of claim 1 wherein the sample is a blood sample, urine sample, plasma or serum sample, nasal swab sample, or a cerebrospinal fluid sample.

11. The method of claim 1 wherein the sample comprises cells.

12. The method of claim 11 wherein the sample comprises human cells.

13. The method of claim 1 wherein the sample is a tissue biopsy.

14. The method of claim 1 wherein aggregation is detected using a system that contains a camera and optionally analyzes images from the camera.

15. The method of claim 14 wherein the system comprises a cell phone.

16. The method of claim 1 wherein the amplification reaction is a loop mediated isothermal amplification reaction.

17. The method of claim 1 wherein the amplification reaction is on a chip.

18. The method of claim 1 wherein the beads and the isolated nucleic acid are added to the chip.

19. The method of claim 1 wherein the isolated nucleic acid comprises genomic DNA.

20. The method of claim 19 wherein the isolated nucleic acid is human genomic DNA.

21.-51. (canceled)
